SEQUENCE LISTING (12 sequences)

SEQ ID NO: 1 - protein, 385 aa, Homo sapiens
MMMMALSKTFGQKPVKFQLEDDGEFYMIGSEVGNYLRMFRGSLYKRYPSLWRRLATVEERKKIVASSHGKKTKPNTKDHGYTTLATSVTLLKASEVEEILDGNDEKYKAVSISTEPPTYLREQKAKRNSQWVPTLPNSSHHLDAVPCSTTINRNRMGRDKKRTFPLCFDDHDPAVIHENASQPEVLVPIRLDMEIDGQKLRDAFTWNMNEKLMTPEMFSEILCDDLDLNPLTFVPAIASAIRQQIESYPTDSILEDQSDQRVIIKLNIHVGNISLVDQFEWDMSEKENSPEKFALKLCSELGLGGEFVTTIAYSIRGQLSWHQKTYAFSENPLPTVEIAIRNTGDADQWCPLLETLTDAEMEKKIRDQDRNTRRMRRLANTAPAW

SEQ ID NO: 2 - DNA, 1717 bp, Homo sapiens
aacgccagcgcctgcgcactgagggcggcctggtcgtcgtctgcggcggcggcggcggctgaggagcccggctgaggcgccagtacccggcccggtccgcatttcgccttccggcttcggtttccctcggcccagcacgccccggccccgccccagccctcctgatccctcgcagcccggctccggccgcccgcctctgccgccgcaatgatgatgatggcgctgagcaagaccttcgggcagaagcccgtgaagttccagctggaggacgacggcgagttctacatgatcggctccgaggtgggaaactacctccgtatgttccgaggttctctgtacaagagatacccctcactctggaggcgactagccactgtggaagagaggaagaaaatagttgcatcgtcacatggtaaaaaaacaaaacctaacactaaggatcacggatacacgactctagccaccagtgtgaccctgttaaaagcctcggaagtggaagagattctggatggcaacgatgagaagtacaaggctgtgtccatcagcacagagccccccacctacctcagggaacagaaggccaagaggaacagccagtgggtacccaccctgcccaacagctcccaccacttagatgccgtgccatgctccacaaccatcaacaggaaccgcatgggccgagacaagaagagaaccttccccctttgctttgatgaccatgacccagctgtgatccatgagaacgcatctcagcccgaggtgctggtccccatccggctggacatggagatcgatgggcagaagctgcgagacgccttcacctggaacatgaatgagaagttgatgacgcctgagatgttttcagaaatcctctgtgacgatctggatttgaacccgctgacgtttgtgccagccatcgcctctgccatcagacagcagatcgagtcctaccccacggacagcatcctggaggaccagtcagaccagcgcgtcatcatcaagctgaacatccatgtgggaaacatttccctggtggaccagtttgagtgggacatgtcagagaaggagaactcaccagagaagtttgccctgaagctgtgctcggagctggggttgggcggggagtttgtcaccaccatcgcatacagcatccggggacagctgagctggcatcagaagacctacgccttcagcgagaaccctctgcccacagtggagattgccatccggaacacgggcgatgcggaccagtggtgcccactgctggagactctgacagacgctgagatggagaagaagatccgcgaccaggacaggaacacgaggcggatgaggcgtcttgccaacacggccccggcctggtaaccagcccatcagcacacggctcccacggagcatctcagaagattgggccgcctctcctccatcttctggcaaggacagaggcgaggggacagcccagcgccatcctgaggatcgggtgggggtggagtgggggcttccaggtggcccttcccggcacacattccatttgttgagccccagtcctgccccccaccccaccctccctacccctccccagtctctggggtcaggaagaaaccttattttaggttgtgttttgtttttgtataggagccccaggcagggctagtaacagtttttaaataaaaggcaacaggtcatgttcaatttcttcaacaaaaaaaaaaaaaaa

SEQ ID NO: 3 - protein, 376 aa, Homo sapiens
MMMMALSKTFGQKPVKFQLEDDGEFYMIGSEVGNYLRMFRGSLYKRYPSLWRRLATVEERKKIVASSHDHGYTTLATSVTLLKASEVEEILDGNDEKYKAVSISTEPPTYLREQKAKRNSQWVPTLPNSSHHLDAVPCSTTINRNRMGRDKKRTFPLCFDDHDPAVIHENASQPEVLVPIRLDMEIDGQKLRDAFTWNMNEKLMTPEMFSEILCDDLDLNPLTFVPAIASAIRQQIESYPTDSILEDQSDQRVIIKLNIHVGNISLVDQFEWDMSEKENSPEKFALKLCSELGLGGEFVTTIAYSIRGQLSWHQKTYAFSENPLPTVEIAIRNTGDADQWCPLLETLTDAEMEKKIRDQDRNTRRMRRLANTAPAW

SEQ ID NO: 4 - DNA, 1690 bp, Homo sapiens
aacgccagcgcctgcgcactgagggcggcctggtcgtcgtctgcggcggcggcggcggctgaggagcccggctgaggcgccagtacccggcccggtccgcatttcgccttccggcttcggtttccctcggcccagcacgccccggccccgccccagccctcctgatccctcgcagcccggctccggccgcccgcctctgccgccgcaatgatgatgatggcgctgagcaagaccttcgggcagaagcccgtgaagttccagctggaggacgacggcgagttctacatgatcggctccgaggtgggaaactacctccgtatgttccgaggttctctgtacaagagatacccctcactctggaggcgactagccactgtggaagagaggaagaaaatagttgcatcgtcacatgatcacggatacacgactctagccaccagtgtgaccctgttaaaagcctcggaagtggaagagattctggatggcaacgatgagaagtacaaggctgtgtccatcagcacagagccccccacctacctcagggaacagaaggccaagaggaacagccagtgggtacccaccctgcccaacagctcccaccacttagatgccgtgccatgctccacaaccatcaacaggaaccgcatgggccgagacaagaagagaaccttccccctttgctttgatgaccatgacccagctgtgatccatgagaacgcatctcagcccgaggtgctggtccccatccggctggacatggagatcgatgggcagaagctgcgagacgccttcacctggaacatgaatgagaagttgatgacgcctgagatgttttcagaaatcctctgtgacgatctggatttgaacccgctgacgtttgtgccagccatcgcctctgccatcagacagcagatcgagtcctaccccacggacagcatcctggaggaccagtcagaccagcgcgtcatcatcaagctgaacatccatgtgggaaacatttccctggtggaccagtttgagtgggacatgtcagagaaggagaactcaccagagaagtttgccctgaagctgtgctcggagctggggttgggcggggagtttgtcaccaccatcgcatacagcatccggggacagctgagctggcatcagaagacctacgccttcagcgagaaccctctgcccacagtggagattgccatccggaacacgggcgatgcggaccagtggtgcccactgctggagactctgacagacgctgagatggagaagaagatccgcgaccaggacaggaacacgaggcggatgaggcgtcttgccaacacggccccggcctggtaaccagcccatcagcacacggctcccacggagcatctcagaagattgggccgcctctcctccatcttctggcaaggacagaggcgaggggacagcccagcgccatcctgaggatcgggtgggggtggagtgggggcttccaggtggcccttcccggcacacattccatttgttgagccccagtcctgccccccaccccaccctccctacccctccccagtctctggggtcaggaagaaaccttattttaggttgtgttttgtttttgtataggagccccaggcagggctagtaacagtttttaaataaaaggcaacaggtcatgttcaatttcttcaacaaaaaaaaaaaaaaa

SEQ ID NO: 5 - protein, 2492 aa, Homo sapiens
MTAEPMSESKLNTLVQKLHDFLAHSSEESEETSSPPRLAMNQNTDKISGSGSNSDMMENSKEEGTSSSEKSKSSGSSRSKRKPSIVTKYVESDDEKPLDDETVNEDASNENSENDITMQSLPKGTVIVQPEPVLNEDKDDFKGPEFRSRSKMKTENLKKRGEDGLHGIVSCTACGQQVNHFQKDSIYRHPSLQVLICKNCFKYYMSDDISRDSDGMDEQCRWCAEGGNLICCDFCHNAFCKKCILRNLGRKELSTIMDENNQWYCYICHPEPLLDLVTACNSVFENLEQLLQQNKKKIKVDSEKSNKVYEHTSRFSPKKTSSNCNGEEKKLDDSCSGSVTYSYSALIVPKEMIKKAKKLIETTANMNSSYVKFLKQATDNSEISSATKLRQLKAFKSVLADIKKAHLALEEDLNSEFRAMDAVNKEKNTKEHKVIDAKFETKARKGEKPCALEKKDISKSEAKLSRKQVDSEHMHQNVPTEEQRTNKSTGGEHKKSDRKEEPQYEPANTSEDLDMDIVSVPSSVPEDIFENLETAMEVQSSVDHQGDGSSGTEQEVESSSVKLNISSKDNRGGIKSKTTAKVTKELYVKLTPVSLSNSPIKGADCQEVPQDKDGYKSCGLNPKLEKCGLGQENSDNEHLVENEVSLLLEESDLRRSPRVKTTPLRRPTETNPVTSNSDEECNETVKEKQKLSVPVRKKDKRNSSDSAIDNPKPNKLPKSKQSETVDQNSDSDEMLAILKEVSRMSHSSSSDTDINEIHTNHKTLYDLKTQAGKDDKGKRKRKSSTSGSDFDTKKGKSAKSSIISKKKRQTQSESSNYDSELEKEIKSMSKIGAARTTKKRIPNTKDFDSSEDEKHSKKGMDNQGHKNLKTSQEGSSDDAERKQERETFSSAEGTVDKDTTIMELRDRLPKKQQASASTDGVDKLSGKEQSFTSLEVRKVAETKEKSKHLKTKTCKKVQDGLSDIAEKFLKKDQSDETSEDDKKQSKKGTEEKKKPSDFKKKVIKMEQQYESSSDGTEKLPEREEICHFPKGIKQIKNGTTDGEKKSKKIRDKTSKKKDELSDYAEKSTGKGDSCDSSEDKKSKNGAYGREKKRCKLLGKSSRKRQDCSSSDTEKYSMKEDGCNSSDKRLKRIELRERRNLSSKRNTKEIQSGSSSSDAEESSEDNKKKKQRTSSKKKAVIVKEKKRNSLRTSTKRKQADITSSSSSDIEDDDQNSIGEGSSDEQKIKPVTENLVLSSHTGFCQSSGDEALSKSVPVTVDDDDDDNDPENRIAKKMLLEEIKANLSSDEDGSSDDEPEEGKKRTGKQNEENPGDEEAKNQVNSESDSDSEESKKPRYRHRLLRHKLTVSDGESGEEKKTKPKEHKEVKGRNRRKVSSEDSEDSDFQESGVSEEVSESEDEQRPRTRSAKKAELEENQRSYKQKKKRRRIKVQEDSSSENKSNSEEEEEEKEEEEEEEEEEEEEEEDENDDSKSPGKGRKKIRKILKDDKLRTETQNALKEEEERRKRIAEREREREKLREVIEIEDASPTKCPITTKLVLDEDEETKEPLVQVHRNMVIKLKPHQVDGVQFMWDCCCESVKKTKKSPGSGCILAHCMGLGKTLQVVSFLHTVLLCDKLDFSTALVVCPLNTALNWMNEFEKWQEGLKDDEKLEVSELATVKRPQERSYMLQRWQEDGGVMIIGYEMYRNLAQGRNVKSRKLKEIFNKALVDPGPDFVVCDEGHILKNEASAVSKAMNSIRSRRRIILTGTPLQNNLIEYHCMVNFIKENLLGSIKEFRNRFINPIQNGQCADSTMVDVRVMKKRAHILYEMLAGCVQRKDYTALTKFLPPKHEYVLAVRMTSIQCKLYQYYLDHLTGVGNNSEGGRGKAGAKLFQDFQMLSRIWTHPWCLQLDYISKENKGYFDEDSMDEFIASDSDETSMSLSSDDYTKKKKKGKKGKKDSSSSGSGSDNDVEVIKVWNSRSRGGGEGNVDETGNNPSVSLKLEESKATSSSNPSSPAPDWYKDFVTDADAEVLEHSGKMVLLFEILRMAEEIGDKVLVFSQSLISLDLIEDFLELASREKTEDKDKPLIYKGEGKWLRNIDYYRLDGSTTAQSRKKWAEEFNDETNVRGRLFIISTKAGSLGINLVAANRVIIFDASWNPSYDIQSIFRVYRFGQTKPVYVYRFLAQGTMEDKIYDRQVTKQSLSFRVVDQQQVERHFTMNELTELYTFEPDLLDDPNSEKKKKRDTPMLPKDTILAELLQIHKEHIVGYHEHDSLLDHKEEEELTEEERKAAWAEYEAEKKGLTMRFNIPTGTNLPPVSFNSQTPYIPFNLGALSAMSNQQLEDLINQGREKVVEATNSVTAVRIQPLEDIISAVWKENMNLSEAQVQALALSRQASQELDVKRREAIYNDVLTKQQMLISCVQRILMNRRLQQQYNQQQQQQMTYQQATLGHLMMPKPPNLIMNPSNYQQIDMRGMYQPVAGGMQPPPLQRAPPPMRSKNPGPSQGKSM

SEQ ID NO: 6 - DNA, 11202 bp, Homo sapiens
aattctcctgcctgagcctcggcccaacaaaatggcggcggcagcggtgtcgctttgtttccgcggctcctgcggcggtggcagtggtagcggcctttgagctgtggggaggttccagcagcagctacagtgacgactaagactccagtgcatttctatcgtaaccgggcgcgggggagcgcagatcggcgcccagcaatcacagaagccgacaaggcgttcaagcgaaaacatgaccgctgagcccatgagtgaaagcaagttgaatacattggtgcagaagcttcatgacttccttgcacactcatcagaagaatctgaagaaacaagttctcctccacgacttgcaatgaatcaaaacacagataaaatcagtggttctggaagtaactctgatatgatggaaaacagcaaggaagagggaactagctcttcagaaaaatccaagtcttcaggatcgtcacgatcaaagaggaaaccttcaattgtaacaaagtatgtagaatcagatgatgaaaaacctttggatgatgaaactgtaaatgaagatgcgtctaatgaaaattcagaaaatgatattactatgcagagcttgccaaaaggtacagtgattgtacagccagagccagtgctgaatgaagacaaagatgattttaaagggcctgaatttagaagcagaagtaaaatgaaaactgaaaatctcaaaaaacgcggagaagatgggcttcatgggattgtgagctgcactgcttgtggacaacaggtcaatcattttcaaaaagattccatttatagacacccttcattgcaagttcttatttgtaagaattgctttaagtattacatgagtgatgatattagccgtgactcagatggaatggatgaacaatgtaggtggtgtgcggaaggtggaaacttgatttgttgtgacttttgccataatgctttctgcaagaaatgcattctacgcaaccttggtcgaaaggagttgtccacaataatggatgaaaacaaccaatggtattgctacatttgtcacccagagcctttgttggacttggtcactgcatgtaacagcgtatttgagaatttagaacagttgttgcagcaaaataagaagaagataaaagttgacagtgaaaagagtaataaagtatatgaacatacatccagattttctccaaagaagactagttcaaattgtaatggagaagaaaagaaattagatgattcctgttctggctctgtaacctactcttattccgcactaattgtgcccaaagagatgattaagaaggcaaaaaaactgattgagaccacagccaacatgaactccagttatgttaaatttttaaagcaggcaacagataattcagaaatcagttctgctacaaaattacgtcagcttaaggcttttaagtctgtgttggctgatattaagaaggctcatcttgcattggaagaagacttaaattccgagtttcgagcgatggatgctgtaaacaaagagaaaaataccaaagagcataaagtcatagatgctaagtttgaaacaaaagcacgaaaaggagaaaaaccttgtgctttggaaaagaaggatatttcaaagtcagaagctaaactttcaagaaaacaggtagatagtgagcacatgcatcagaatgttccaacagaggaacaaagaacaaataaaagtaccggtggtgaacataagaaatctgatagaaaagaagaacctcaatatgaacctgccaacacttctgaagatttagacatggatattgtgtctgttccttcctcagttccagaagacatttttgagaatcttgagactgctatggaagttcagagttcagttgatcatcaaggggatggcagcagtggaactgaacaagaagtggagagttcatctgtaaaattaaatatttcttcaaaagacaacagaggaggtattaaatcaaaaactacagctaaagtaacaaaagaattatatgttaaactcactcctgtttccctttctaattccccaattaaaggtgctgattgtcaggaagttccacaagataaagatggctataaaagttgtggtctgaaccccaagttagagaaatgtggacttggacaggaaaacagtgataatgagcatttggttgaaaatgaagtttcattacttttagaggaatctgatcttcgaagatccccacgtgtaaagactacacccttgaggcgaccgacagaaactaaccctgtaacatctaattcagatgaagaatgtaatgaaacagttaaggagaaacaaaaactatcagttccagtgagaaaaaaggataagcgtaattcttctgacagtgctatagataatcctaagcctaataaattgccaaaatctaagcaatcagagactgtggatcaaaattcagattctgatgaaatgctagcaatcctcaaagaggtgagcaggatgagtcacagttcttcttcagatactgatattaatgaaattcatacaaaccataagactttgtatgatttaaagactcaggcggggaaagatgataaaggaaaaaggaaacgaaaaagttctacatctggctcagattttgatactaaaaagggcaaatcagctaagagctctataatttctaaaaagaaacgacaaacccagtctgagtcttctaattatgactcagaattagaaaaagagataaagagcatgagtaaaattggtgctgccagaaccaccaaaaaaagaattccaaatacaaaagattttgactcttctgaagatgagaaacacagcaaaaaaggaatggataatcaagggcacaaaaatttgaagacctcacaagaaggatcatctgatgatgctgaaagaaaacaagagagagagactttctcttcagcagaaggcacagttgataaagacacgaccatcatggaattaagagatcgacttcctaagaagcagcaagcaagtgcttccactgatggtgtcgataagctttctgggaaagagcagagttttacttctttggaagttagaaaagttgctgaaactaaagaaaagagcaagcatctcaaaaccaaaacatgtaaaaaagtacaggatggcttatctgatattgcagagaaattcctaaagaaagaccagagcgatgaaacttctgaagatgataaaaagcagagcaaaaagggaactgaagaaaaaaagaaaccttcagactttaagaaaaaagtaattaaaatggaacaacagtatgaatcttcatctgatggcactgaaaagttacctgagcgagaagaaatttgtcattttcctaagggcataaaacaaattaagaatggaacaactgatggagaaaagaaaagtaaaaaaataagagataaaacttctaaaaagaaggatgaattatctgattatgctgagaagtcaacagggaaaggagatagttgtgactcttcagaggataaaaagagtaagaatggagcatatggtagagagaagaaaaggtgcaagttgcttggaaagagttcaaggaagagacaagattgttcatcatctgatactgagaaatattccatgaaagaagatggttgtaactcttctgataagagactgaaaagaatagaattgagggaaagaagaaatttaagttcaaagagaaatactaaggaaatacaaagtggctcatcatcatctgatgctgaggaaagttctgaagataataaaaagaagaagcaaagaacttcatctaaaaagaaggcagtcattgtcaaggagaaaaagagaaactccctaagaacaagcactaaaaggaagcaagctgacattacatcctcatcttcttctgatatagaagatgatgatcagaattctataggtgagggaagcagcgatgaacagaaaattaagcctgtgactgaaaatttagtgctgtcttcacatactggattttgccaatcttcaggagatgaagccttatctaaatcagtgcctgtcacagtggatgatgatgatgacgacaatgatcctgagaatagaattgccaagaagatgcttttagaagaaattaaagccaatctttcctctgatgaggatggatcttcagatgatgagccagaagaagggaaaaaaagaactggaaaacaaaatgaagaaaacccaggagatgaggaagcaaaaaatcaagtcaattctgaatcagattcagattctgaagaatctaagaagccaagatacagacataggcttttgcggcacaaattgactgtgagtgacggagaatctggagaagaaaaaaagacaaagcctaaagagcataaagaagtcaaaggcagaaacagaagaaaggtgagcagtgaagattcagaagattctgattttcaggaatcaggagttagtgaagaagttagtgaatccgaagatgaacagcggcccagaacaaggtctgcaaagaaagcagagttggaagaaaatcagcggagctataaacagaaaaagaaaaggcgacgtattaaggttcaagaagattcatccagtgaaaacaagagtaattctgaggaagaagaggaggaaaaagaagaggaggaggaagaggaggaggaggaggaagaggaggaggaagatgaaaatgatgattccaagtctcctggaaaaggcagaaagaaaattcggaagattcttaaagatgataaactgagaacagaaacacaaaatgctcttaaggaagaggaagagagacgaaaacgtattgctgagagggagcgtgagcgagaaaaattgagagaggtgatagaaattgaagatgcttcacccaccaagtgtccaataacaaccaagttggttttagatgaagatgaagaaaccaaagaacctttagtgcaggttcatagaaatatggttatcaaattgaaaccccatcaagtagatggtgttcagtttatgtgggattgctgctgtgagtctgtgaaaaaaacaaagaaatctccaggttcaggatgcattcttgcccactgtatgggccttggtaagactttacaggtggtaagttttcttcatacagttcttttgtgtgacaaactggatttcagcacggcgttagtggtttgtcctcttaatactgctttgaattggatgaatgaatttgagaagtggcaagagggattaaaagatgatgagaagcttgaggtttctgaattagcaactgtgaaacgtcctcaggagagaagctacatgctgcagaggtggcaagaagatggtggtgttatgatcataggctatgagatgtatagaaatcttgctcaaggaaggaatgtgaagagtcggaaacttaaagaaatatttaacaaagctttggttgatccaggccctgattttgttgtttgtgatgaaggccatattctaaaaaatgaagcatctgctgtttctaaagctatgaattctatacgatcaaggaggaggattattttaacaggaacaccacttcaaaataacctaattgagtatcattgtatggttaattttatcaaggaaaatttacttggatccattaaggagttcaggaatagatttataaatccaattcaaaatggtcagtgtgcagattctaccatggtagatgtcagagtgatgaaaaaacgtgctcacattctctatgagatgttagctggatgtgttcagaggaaagattatacagcattaacaaaattcttgcctccaaaacacgaatatgtgttagctgtgagaatgacttctattcagtgcaagctctatcagtactacttagatcacttaacaggtgtgggcaataatagtgaaggtggaagaggaaaggcaggtgcaaagcttttccaagattttcagatgttaagtagaatatggactcatccttggtgtttgcagctagactacattagcaaagaaaataagggttattttgatgaagacagtatggatgaatttatagcctcagattctgatgaaacctccatgagtttaagctccgatgattatacaaaaaagaagaaaaaagggaaaaaggggaaaaaagatagtagctcaagtggaagtggcagtgacaatgatgttgaagtgattaaggtctggaattcaagatctcggggaggtggtgaaggaaatgtggatgaaacaggaaacaatccttctgtttctttaaaactggaagaaagtaaagctacttcttcttctaatccaagcagcccagctccagactggtacaaagattttgttacagatgctgatgctgaggttttagagcattctgggaaaatggtacttctctttgaaattcttcgaatggcagaggaaattggggataaagtccttgttttcagccagtccctcatatctctggacttgattgaagattttcttgaattagctagtagggagaagacagaagataaagataaaccccttatttataaaggtgaggggaagtggcttcgaaacattgactattaccgtttagatggttccactactgcacagtcaaggaagaagtgggctgaagaatttaatgatgaaactaatgtgagaggacgattatttatcatttctactaaagcaggatctctaggaattaatctggtagctgctaatcgagtaattatattcgacgcttcttggaatccatcttatgacatccagagtatattcagagtttatcgctttggacaaactaagcctgtttatgtatataggttcttagctcagggaaccatggaagataagatttatgatcggcaagtaactaagcagtcactgtcttttcgagttgttgatcagcagcaggtggagcgtcattttactatgaatgagcttactgaactttatacttttgagccagacttattagatgaccctaattcagaaaagaagaagaagagggatactcccatgctgccaaaggataccatacttgcagagctccttcagatacataaagaacacattgtaggataccatgaacatgattctcttttggaccacaaagaagaagaagagttgactgaagaagaaagaaaagcagcttgggctgagtatgaagcagagaagaagggactgaccatgcgtttcaacataccaactgggaccaatttaccccctgtcagtttcaactctcaaactccttatattcctttcaatttgggagccctgtcagcaatgagtaatcaacagctggaggacctcattaatcaaggaagagaaaaagttgtagaagcaacaaacagtgtgacagcagtgaggattcaacctcttgaggatataatttcagctgtatggaaggagaacatgaatctctcagaggcccaagtacaggcgttagcattaagtagacaagccagccaggagcttgatgttaaacgaagagaagcaatctacaatgatgtattgacaaaacaacagatgttaatcagctgtgttcagcgaatacttatgaacagaaggctccagcagcagtacaatcagcagcaacagcaacaaatgacttatcaacaagcaacactgggtcacctcatgatgccaaagcccccaaatttgatcatgaatccttctaactaccagcagattgatatgagaggaatgtatcagccagtggctggtggtatgcagccaccaccattacagcgtgcaccacccccaatgagaagcaaaaatccaggaccttcccaagggaaatcaatgtgattttgcactaaaagcttaatggattgttaaaatcatagaaagatcttttatttttttaggaatcaatgacttaacagaactcaactgtataaatagtttggtccccttaaatgccaatcttccatattagttttactttttttttttttaaatagggcataccatttcttcctgacatttgtcagtgatgttgcctagaatcttcttacacacgctgagtacagaagatatttcaaattgttttcagtgaaaacaagtccttccataatagtaacaactccacagatttcctctctaaatttttatgcctgcttttagcaaccataaaattgtcataaaattaataaatttaggaaagaataaagatttatatattcattctttacatataaaaacacacagctgagttcttagagttgattcctcaagttatgaaatacttttgtacttaatccatttcttgattaaagtgattgaaatggttttaatgttcttttgactgaagtctgaaactgggctcctgctttattgtctctgtgactgaaagttagaaactgagggttatctttgacacagaattgtgtgcaatattcttaaatactactgctctaaaagttggagaagtcttgcagttatcttagcattgtataaacagccttaagtatagcctaagaagagaattcctttttcttctttagtccttctgccattttttattttcagttatatgtgctgaaataattactggtaaaatttcagggttgtggattatcttccacacatgaattttctctctcctggcacgaatataaagcacatctcttaactgcatggtgccagtgctaatgcttcatcctgttgctggcagtgggatgtggacttagaaaatcaagttctagcattttagtaggttaacactgaagttgtggttgttaggttcacaccctgttttataaacaacatcaaaatggcagaaccattgctgactttaggttcacatgaggaatgtacttttaacaattcccagtactatcagtattgtgaaataattcctctgaaagataagaatcactggcttctatgcgcttcttttctctcatcatcatgttcttttaccccagtttccttacatttttttaaattgtttcagagtttgttttttttttagtttagattgtgaggcaattattaaatcaaaattaattcatccaatacccctttactagaagttttactagaaaatgtattacattttattttttcttaatccagttctgcaaaaatgacctataaatttattcatgtacaattttggttacttgaattgttaaagaaaacattgtttttgactatgggagtcaactcaacatggcagaaccatttttgagatgatgatacaacaggtagtgaaacagcttaagaattccaaaaaaaaaaaaaaaaaaaaaaaaaagaaaactgggtttgggctttgctttaggtatcactggattagaatgagtttaacattagctaaaactgctttgagttgtttggatgattaagagattgccatttttatcttggaagaactagtggtaaaacatccaagagcactaggattgtgatacagaatttgtgaggtttggtggatccacgcccctctcccccactttcccatgatgaaatatcactaataaatcctgtatatttagatattatgctagccatgtaatcagatttatttaattgggtggggcaggtgtgtatttactttagaaaaaatgaaaaagacaagatttatgagaaatatttgaaggcagtacactctggccaactgttaccagttggtatttctacaagttcagaatattttaaacctgatttactagacctgggaattttcaacatggtctaattatttactcaaagacatagatgtgaaaattttaggcaaccttctaaatctttttcaccatggatgaaactataacttaaagaataatacttagaagggttaattggaaatcagagtttgaaataaaacttggaccactttgtatacactcttctcacttgacattttagctatataatatgtactttgagtataacatcaagctttaacaaatatttaaagacaaaaaaatcacgtcagtaaaatactaaaaggctcatttttatatttgttttagatgttttaaatagttgcaatggattaaaaatgatgatttaaaatgttgcttgtaatacagttttgcctgctaaattctccacattttgtaacctgttttatttctttgggtgtaaagcgtttttgcttagtattgtgatattgtatatgttttgtcccagttgtatagtaatgtttcagtccatcatccagctttggctgctgaaatcatacagctgtgaagacttgcctttgtttctgttagactgcttttcagttctgtattgagtatcttaagtactgtagaaaagatgtcacttcttcctttaaggctgttttgtaatatatataaggactggaattgtgtttttaaagaaaagcattcaagtatgacaatatactatctgtgttttcaccattcaaagtgctgtttagtagttgaaacttaaactatttaatgtcatttaataaagtgaccaaaatgtgttgtgctctttattgtattttcacagctttgaaaatctgtgcacatactgtttcatagaaaatgtatagcttttgttgtcctatataatggtggttcttttgcacatttagttatttaatattgagaggtcacgaagtttggttattgaatctgttatatactaaattctgtaaagggagatctctcatctcaaaaagaatttacataccaggaagtccatgtgtgtttgtgttagttttggatgtctttgtgtaatccagccccatttcctgtttcccaacagctgtaacactcattttaagtcaagcagggctaccaacccacacttgatagaaaagctgcttaccattcagaagcttccttattacctggcctccaaatgagctgaatattttgtagccttcccttagctatgttcattttccctccattatcataaaatcagatcgatatttatgtgccccaaacaaaactttaagagcagttacattctgtcccagtagcccttgtttcctttgagagtagcatgttgtgaggctatagagacttattctaccagtaaaacaggtcaatccttttacatgtttattatactaaaaattatgttcagggtatttactactttatttcaccagactcagtctcaagtgacttggctatctccaaatcagatctacccttagagaataaacatttttctaccgttattttttttcaagtctataatctgagccagtcccaaaggagtgatcaagtttcagaaatgctttcatcttcacaacattttatatatactattatatggggtgaataaagttttaaatccgaaatataaaaaaaaaaaaaaaaaaa

SEQ ID NO: 7 - protein, 2454 aa, Homo sapiens
MTAEPMSESKLNTLVQKLHDFLAHSSEESEETSSPPRLAMNQNTDKISGSGSNSDMMENSKEEGTSSSEKSKSSGSSRSKRKPSIVTKYVESDDEKPLDDETVNEDASNENSENDITMQSLPKEDGLHGIVSCTACGQQVNHFQKDSIYRHPSLQVLICKNCFKYYMSDDISRDSDGMDEQCRWCAEGGNLICCDFCHNAFCKKCILRNLGRKELSTIMDENNQWYCYICHPEPLLDLVTACNSVFENLEQLLQQNKKKIKVDSEKSNKVYEHTSRFSPKKTSSNCNGEEKKLDDSCSGSVTYSYSALIVPKEMIKKAKKLIETTANMNSSYVKFLKQATDNSEISSATKLRQLKAFKSVLADIKKAHLALEEDLNSEFRAMDAVNKEKNTKEHKVIDAKFETKARKGEKPCALEKKDISKSEAKLSRKQVDSEHMHQNVPTEEQRTNKSTGGEHKKSDRKEEPQYEPANTSEDLDMDIVSVPSSVPEDIFENLETAMEVQSSVDHQGDGSSGTEQEVESSSVKLNISSKDNRGGIKSKTTAKVTKELYVKLTPVSLSNSPIKGADCQEVPQDKDGYKSCGLNPKLEKCGLGQENSDNEHLVENEVSLLLEESDLRRSPRVKTTPLRRPTETNPVTSNSDEECNETVKEKQKLSVPVRKKDKRNSSDSAIDNPKPNKLPKSKQSETVDQNSDSDEMLAILKEVSRMSHSSSSDTDINEIHTNHKTLYDLKTQAGKDDKGKRKRKSSTSGSDFDTKKGKSAKSSIISKKKRQTQSESSNYDSELEKEIKSMSKIGAARTTKKRIPNTKDFDSSEDEKHSKKGMDNQGHKNLKTSQEGSSDDAERKQERETFSSAEGTVDKDTTIMELRDRLPKKQQASASTDGVDKLSGKEQSFTSLEVRKVAETKEKSKHLKTKTCKKVQDGLSDIAEKFLKKDQSDETSEDDKKQSKKGTEEKKKPSDFKKKVIKMEQQYESSSDGTEKLPEREEICHFPKGIKQIKNGTTDGEKKSKKIRDKTSKKKDELSDYAEKSTGKGDSCDSSEDKKSKNGAYGREKKRCKLLGKSSRKRQDCSSSDTEKYSMKEDGCNSSDKRLKRIELRERRNLSSKRNTKEIQSGSSSSDAEESSEDNKKKKQRTSSKKKAVIVKEKKRNSLRTSTKRKQADITSSSSSDIEDDDQNSIGEGSSDEQKIKPVTENLVLSSHTGFCQSSGDEALSKSVPVTVDDDDDDNDPENRIAKKMLLEEIKANLSSDEDGSSDDEPEEGKKRTGKQNEENPGDEEAKNQVNSESDSDSEESKKPRYRHRLLRHKLTVSDGESGEEKKTKPKEHKEVKGRNRRKVSSEDSEDSDFQESGVSEEVSESEDEQRPRTRSAKKAELEENQRSYKQKKKRRRIKVQEDSSSENKSNSEEEEEEKEEEEEEEEEEEEEEEDENDDSKSPGKGRKKIRKILKDDKLRTETQNALKEEEERRKRIAEREREREKLREVIEIEDASPTKCPITTKLVLDEDEETKEPLVQVHRNMVIKLKPHQVDGVQFMWDCCCESVKKTKKSPGSGCILAHCMGLGKTLQVVSFLHTVLLCDKLDFSTALVVCPLNTALNWMNEFEKWQEGLKDDEKLEVSELATVKRPQERSYMLQRWQEDGGVMIIGYEMYRNLAQGRNVKSRKLKEIFNKALVDPGPDFVVCDEGHILKNEASAVSKAMNSIRSRRRIILTGTPLQNNLIEYHCMVNFIKENLLGSIKEFRNRFINPIQNGQCADSTMVDVRVMKKRAHILYEMLAGCVQRKDYTALTKFLPPKHEYVLAVRMTSIQCKLYQYYLDHLTGVGNNSEGGRGKAGAKLFQDFQMLSRIWTHPWCLQLDYISKENKGYFDEDSMDEFIASDSDETSMSLSSDDYTKKKKKGKKGKKDSSSSGSGSDNDVEVIKVWNSRSRGGGEGNVDETGNNPSVSLKLEESKATSSSNPSSPAPDWYKDFVTDADAEVLEHSGKMVLLFEILRMAEEIGDKVLVFSQSLISLDLIEDFLELASREKTEDKDKPLIYKGEGKWLRNIDYYRLDGSTTAQSRKKWAEEFNDETNVRGRLFIISTKAGSLGINLVAANRVIIFDASWNPSYDIQSIFRVYRFGQTKPVYVYRFLAQGTMEDKIYDRQVTKQSLSFRVVDQQQVERHFTMNELTELYTFEPDLLDDPNSEKKKKRDTPMLPKDTILAELLQIHKEHIVGYHEHDSLLDHKEEEELTEEERKAAWAEYEAEKKGLTMRFNIPTGTNLPPVSFNSQTPYIPFNLGALSAMSNQQLEDLINQGREKVVEATNSVTAVRIQPLEDIISAVWKENMNLSEAQVQALALSRQASQELDVKRREAIYNDVLTKQQMLISCVQRILMNRRLQQQYNQQQQQQMTYQQATLGHLMMPKPPNLIMNPSNYQQIDMRGMYQPVAGGMQPPPLQRAPPPMRSKNPGPSQGKSM

SEQ ID NO: 8 - DNA, 11088 bp, Homo sapiens
aattctcctgcctgagcctcggcccaacaaaatggcggcggcagcggtgtcgctttgtttccgcggctcctgcggcggtggcagtggtagcggcctttgagctgtggggaggttccagcagcagctacagtgacgactaagactccagtgcatttctatcgtaaccgggcgcgggggagcgcagatcggcgcccagcaatcacagaagccgacaaggcgttcaagcgaaaacatgaccgctgagcccatgagtgaaagcaagttgaatacattggtgcagaagcttcatgacttccttgcacactcatcagaagaatctgaagaaacaagttctcctccacgacttgcaatgaatcaaaacacagataaaatcagtggttctggaagtaactctgatatgatggaaaacagcaaggaagagggaactagctcttcagaaaaatccaagtcttcaggatcgtcacgatcaaagaggaaaccttcaattgtaacaaagtatgtagaatcagatgatgaaaaacctttggatgatgaaactgtaaatgaagatgcgtctaatgaaaattcagaaaatgatattactatgcagagcttgccaaaagaagatgggcttcatgggattgtgagctgcactgcttgtggacaacaggtcaatcattttcaaaaagattccatttatagacacccttcattgcaagttcttatttgtaagaattgctttaagtattacatgagtgatgatattagccgtgactcagatggaatggatgaacaatgtaggtggtgtgcggaaggtggaaacttgatttgttgtgacttttgccataatgctttctgcaagaaatgcattctacgcaaccttggtcgaaaggagttgtccacaataatggatgaaaacaaccaatggtattgctacatttgtcacccagagcctttgttggacttggtcactgcatgtaacagcgtatttgagaatttagaacagttgttgcagcaaaataagaagaagataaaagttgacagtgaaaagagtaataaagtatatgaacatacatccagattttctccaaagaagactagttcaaattgtaatggagaagaaaagaaattagatgattcctgttctggctctgtaacctactcttattccgcactaattgtgcccaaagagatgattaagaaggcaaaaaaactgattgagaccacagccaacatgaactccagttatgttaaatttttaaagcaggcaacagataattcagaaatcagttctgctacaaaattacgtcagcttaaggcttttaagtctgtgttggctgatattaagaaggctcatcttgcattggaagaagacttaaattccgagtttcgagcgatggatgctgtaaacaaagagaaaaataccaaagagcataaagtcatagatgctaagtttgaaacaaaagcacgaaaaggagaaaaaccttgtgctttggaaaagaaggatatttcaaagtcagaagctaaactttcaagaaaacaggtagatagtgagcacatgcatcagaatgttccaacagaggaacaaagaacaaataaaagtaccggtggtgaacataagaaatctgatagaaaagaagaacctcaatatgaacctgccaacacttctgaagatttagacatggatattgtgtctgttccttcctcagttccagaagacatttttgagaatcttgagactgctatggaagttcagagttcagttgatcatcaaggggatggcagcagtggaactgaacaagaagtggagagttcatctgtaaaattaaatatttcttcaaaagacaacagaggaggtattaaatcaaaaactacagctaaagtaacaaaagaattatatgttaaactcactcctgtttccctttctaattccccaattaaaggtgctgattgtcaggaagttccacaagataaagatggctataaaagttgtggtctgaaccccaagttagagaaatgtggacttggacaggaaaacagtgataatgagcatttggttgaaaatgaagtttcattacttttagaggaatctgatcttcgaagatccccacgtgtaaagactacacccttgaggcgaccgacagaaactaaccctgtaacatctaattcagatgaagaatgtaatgaaacagttaaggagaaacaaaaactatcagttccagtgagaaaaaaggataagcgtaattcttctgacagtgctatagataatcctaagcctaataaattgccaaaatctaagcaatcagagactgtggatcaaaattcagattctgatgaaatgctagcaatcctcaaagaggtgagcaggatgagtcacagttcttcttcagatactgatattaatgaaattcatacaaaccataagactttgtatgatttaaagactcaggcggggaaagatgataaaggaaaaaggaaacgaaaaagttctacatctggctcagattttgatactaaaaagggcaaatcagctaagagctctataatttctaaaaagaaacgacaaacccagtctgagtcttctaattatgactcagaattagaaaaagagataaagagcatgagtaaaattggtgctgccagaaccaccaaaaaaagaattccaaatacaaaagattttgactcttctgaagatgagaaacacagcaaaaaaggaatggataatcaagggcacaaaaatttgaagacctcacaagaaggatcatctgatgatgctgaaagaaaacaagagagagagactttctcttcagcagaaggcacagttgataaagacacgaccatcatggaattaagagatcgacttcctaagaagcagcaagcaagtgcttccactgatggtgtcgataagctttctgggaaagagcagagttttacttctttggaagttagaaaagttgctgaaactaaagaaaagagcaagcatctcaaaaccaaaacatgtaaaaaagtacaggatggcttatctgatattgcagagaaattcctaaagaaagaccagagcgatgaaacttctgaagatgataaaaagcagagcaaaaagggaactgaagaaaaaaagaaaccttcagactttaagaaaaaagtaattaaaatggaacaacagtatgaatcttcatctgatggcactgaaaagttacctgagcgagaagaaatttgtcattttcctaagggcataaaacaaattaagaatggaacaactgatggagaaaagaaaagtaaaaaaataagagataaaacttctaaaaagaaggatgaattatctgattatgctgagaagtcaacagggaaaggagatagttgtgactcttcagaggataaaaagagtaagaatggagcatatggtagagagaagaaaaggtgcaagttgcttggaaagagttcaaggaagagacaagattgttcatcatctgatactgagaaatattccatgaaagaagatggttgtaactcttctgataagagactgaaaagaatagaattgagggaaagaagaaatttaagttcaaagagaaatactaaggaaatacaaagtggctcatcatcatctgatgctgaggaaagttctgaagataataaaaagaagaagcaaagaacttcatctaaaaagaaggcagtcattgtcaaggagaaaaagagaaactccctaagaacaagcactaaaaggaagcaagctgacattacatcctcatcttcttctgatatagaagatgatgatcagaattctataggtgagggaagcagcgatgaacagaaaattaagcctgtgactgaaaatttagtgctgtcttcacatactggattttgccaatcttcaggagatgaagccttatctaaatcagtgcctgtcacagtggatgatgatgatgacgacaatgatcctgagaatagaattgccaagaagatgcttttagaagaaattaaagccaatctttcctctgatgaggatggatcttcagatgatgagccagaagaagggaaaaaaagaactggaaaacaaaatgaagaaaacccaggagatgaggaagcaaaaaatcaagtcaattctgaatcagattcagattctgaagaatctaagaagccaagatacagacataggcttttgcggcacaaattgactgtgagtgacggagaatctggagaagaaaaaaagacaaagcctaaagagcataaagaagtcaaaggcagaaacagaagaaaggtgagcagtgaagattcagaagattctgattttcaggaatcaggagttagtgaagaagttagtgaatccgaagatgaacagcggcccagaacaaggtctgcaaagaaagcagagttggaagaaaatcagcggagctataaacagaaaaagaaaaggcgacgtattaaggttcaagaagattcatccagtgaaaacaagagtaattctgaggaagaagaggaggaaaaagaagaggaggaggaagaggaggaggaggaggaagaggaggaggaagatgaaaatgatgattccaagtctcctggaaaaggcagaaagaaaattcggaagattcttaaagatgataaactgagaacagaaacacaaaatgctcttaaggaagaggaagagagacgaaaacgtattgctgagagggagcgtgagcgagaaaaattgagagaggtgatagaaattgaagatgcttcacccaccaagtgtccaataacaaccaagttggttttagatgaagatgaagaaaccaaagaacctttagtgcaggttcatagaaatatggttatcaaattgaaaccccatcaagtagatggtgttcagtttatgtgggattgctgctgtgagtctgtgaaaaaaacaaagaaatctccaggttcaggatgcattcttgcccactgtatgggccttggtaagactttacaggtggtaagttttcttcatacagttcttttgtgtgacaaactggatttcagcacggcgttagtggtttgtcctcttaatactgctttgaattggatgaatgaatttgagaagtggcaagagggattaaaagatgatgagaagcttgaggtttctgaattagcaactgtgaaacgtcctcaggagagaagctacatgctgcagaggtggcaagaagatggtggtgttatgatcataggctatgagatgtatagaaatcttgctcaaggaaggaatgtgaagagtcggaaacttaaagaaatatttaacaaagctttggttgatccaggccctgattttgttgtttgtgatgaaggccatattctaaaaaatgaagcatctgctgtttctaaagctatgaattctatacgatcaaggaggaggattattttaacaggaacaccacttcaaaataacctaattgagtatcattgtatggttaattttatcaaggaaaatttacttggatccattaaggagttcaggaatagatttataaatccaattcaaaatggtcagtgtgcagattctaccatggtagatgtcagagtgatgaaaaaacgtgctcacattctctatgagatgttagctggatgtgttcagaggaaagattatacagcattaacaaaattcttgcctccaaaacacgaatatgtgttagctgtgagaatgacttctattcagtgcaagctctatcagtactacttagatcacttaacaggtgtgggcaataatagtgaaggtggaagaggaaaggcaggtgcaaagcttttccaagattttcagatgttaagtagaatatggactcatccttggtgtttgcagctagactacattagcaaagaaaataagggttattttgatgaagacagtatggatgaatttatagcctcagattctgatgaaacctccatgagtttaagctccgatgattatacaaaaaagaagaaaaaagggaaaaaggggaaaaaagatagtagctcaagtggaagtggcagtgacaatgatgttgaagtgattaaggtctggaattcaagatctcggggaggtggtgaaggaaatgtggatgaaacaggaaacaatccttctgtttctttaaaactggaagaaagtaaagctacttcttcttctaatccaagcagcccagctccagactggtacaaagattttgttacagatgctgatgctgaggttttagagcattctgggaaaatggtacttctctttgaaattcttcgaatggcagaggaaattggggataaagtccttgttttcagccagtccctcatatctctggacttgattgaagattttcttgaattagctagtagggagaagacagaagataaagataaaccccttatttataaaggtgaggggaagtggcttcgaaacattgactattaccgtttagatggttccactactgcacagtcaaggaagaagtgggctgaagaatttaatgatgaaactaatgtgagaggacgattatttatcatttctactaaagcaggatctctaggaattaatctggtagctgctaatcgagtaattatattcgacgcttcttggaatccatcttatgacatccagagtatattcagagtttatcgctttggacaaactaagcctgtttatgtatataggttcttagctcagggaaccatggaagataagatttatgatcggcaagtaactaagcagtcactgtcttttcgagttgttgatcagcagcaggtggagcgtcattttactatgaatgagcttactgaactttatacttttgagccagacttattagatgaccctaattcagaaaagaagaagaagagggatactcccatgctgccaaaggataccatacttgcagagctccttcagatacataaagaacacattgtaggataccatgaacatgattctcttttggaccacaaagaagaagaagagttgactgaagaagaaagaaaagcagcttgggctgagtatgaagcagagaagaagggactgaccatgcgtttcaacataccaactgggaccaatttaccccctgtcagtttcaactctcaaactccttatattcctttcaatttgggagccctgtcagcaatgagtaatcaacagctggaggacctcattaatcaaggaagagaaaaagttgtagaagcaacaaacagtgtgacagcagtgaggattcaacctcttgaggatataatttcagctgtatggaaggagaacatgaatctctcagaggcccaagtacaggcgttagcattaagtagacaagccagccaggagcttgatgttaaacgaagagaagcaatctacaatgatgtattgacaaaacaacagatgttaatcagctgtgttcagcgaatacttatgaacagaaggctccagcagcagtacaatcagcagcaacagcaacaaatgacttatcaacaagcaacactgggtcacctcatgatgccaaagcccccaaatttgatcatgaatccttctaactaccagcagattgatatgagaggaatgtatcagccagtggctggtggtatgcagccaccaccattacagcgtgcaccacccccaatgagaagcaaaaatccaggaccttcccaagggaaatcaatgtgattttgcactaaaagcttaatggattgttaaaatcatagaaagatcttttatttttttaggaatcaatgacttaacagaactcaactgtataaatagtttggtccccttaaatgccaatcttccatattagttttactttttttttttttaaatagggcataccatttcttcctgacatttgtcagtgatgttgcctagaatcttcttacacacgctgagtacagaagatatttcaaattgttttcagtgaaaacaagtccttccataatagtaacaactccacagatttcctctctaaatttttatgcctgcttttagcaaccataaaattgtcataaaattaataaatttaggaaagaataaagatttatatattcattctttacatataaaaacacacagctgagttcttagagttgattcctcaagttatgaaatacttttgtacttaatccatttcttgattaaagtgattgaaatggttttaatgttcttttgactgaagtctgaaactgggctcctgctttattgtctctgtgactgaaagttagaaactgagggttatctttgacacagaattgtgtgcaatattcttaaatactactgctctaaaagttggagaagtcttgcagttatcttagcattgtataaacagccttaagtatagcctaagaagagaattcctttttcttctttagtccttctgccattttttattttcagttatatgtgctgaaataattactggtaaaatttcagggttgtggattatcttccacacatgaattttctctctcctggcacgaatataaagcacatctcttaactgcatggtgccagtgctaatgcttcatcctgttgctggcagtgggatgtggacttagaaaatcaagttctagcattttagtaggttaacactgaagttgtggttgttaggttcacaccctgttttataaacaacatcaaaatggcagaaccattgctgactttaggttcacatgaggaatgtacttttaacaattcccagtactatcagtattgtgaaataattcctctgaaagataagaatcactggcttctatgcgcttcttttctctcatcatcatgttcttttaccccagtttccttacatttttttaaattgtttcagagtttgttttttttttagtttagattgtgaggcaattattaaatcaaaattaattcatccaatacccctttactagaagttttactagaaaatgtattacattttattttttcttaatccagttctgcaaaaatgacctataaatttattcatgtacaattttggttacttgaattgttaaagaaaacattgtttttgactatgggagtcaactcaacatggcagaaccatttttgagatgatgatacaacaggtagtgaaacagcttaagaattccaaaaaaaaaaaaaaaaaaaaaaaaaagaaaactgggtttgggctttgctttaggtatcactggattagaatgagtttaacattagctaaaactgctttgagttgtttggatgattaagagattgccatttttatcttggaagaactagtggtaaaacatccaagagcactaggattgtgatacagaatttgtgaggtttggtggatccacgcccctctcccccactttcccatgatgaaatatcactaataaatcctgtatatttagatattatgctagccatgtaatcagatttatttaattgggtggggcaggtgtgtatttactttagaaaaaatgaaaaagacaagatttatgagaaatatttgaaggcagtacactctggccaactgttaccagttggtatttctacaagttcagaatattttaaacctgatttactagacctgggaattttcaacatggtctaattatttactcaaagacatagatgtgaaaattttaggcaaccttctaaatctttttcaccatggatgaaactataacttaaagaataatacttagaagggttaattggaaatcagagtttgaaataaaacttggaccactttgtatacactcttctcacttgacattttagctatataatatgtactttgagtataacatcaagctttaacaaatatttaaagacaaaaaaatcacgtcagtaaaatactaaaaggctcatttttatatttgttttagatgttttaaatagttgcaatggattaaaaatgatgatttaaaatgttgcttgtaatacagttttgcctgctaaattctccacattttgtaacctgttttatttctttgggtgtaaagcgtttttgcttagtattgtgatattgtatatgttttgtcccagttgtatagtaatgtttcagtccatcatccagctttggctgctgaaatcatacagctgtgaagacttgcctttgtttctgttagactgcttttcagttctgtattgagtatcttaagtactgtagaaaagatgtcacttcttcctttaaggctgttttgtaatatatataaggactggaattgtgtttttaaagaaaagcattcaagtatgacaatatactatctgtgttttcaccattcaaagtgctgtttagtagttgaaacttaaactatttaatgtcatttaataaagtgaccaaaatgtgttgtgctctttattgtattttcacagctttgaaaatctgtgcacatactgtttcatagaaaatgtatagcttttgttgtcctatataatggtggttcttttgcacatttagttatttaatattgagaggtcacgaagtttggttattgaatctgttatatactaaattctgtaaagggagatctctcatctcaaaaagaatttacataccaggaagtccatgtgtgtttgtgttagttttggatgtctttgtgtaatccagccccatttcctgtttcccaacagctgtaacactcattttaagtcaagcagggctaccaacccacacttgatagaaaagctgcttaccattcagaagcttccttattacctggcctccaaatgagctgaatattttgtagccttcccttagctatgttcattttccctccattatcataaaatcagatcgatatttatgtgccccaaacaaaactttaagagcagttacattctgtcccagtagcccttgtttcctttgagagtagcatgttgtgaggctatagagacttattctaccagtaaaacaggtcaatccttttacatgtttattatactaaaaattatgttcagggtatttactactttatttcaccagactcagtctcaagtgacttggctatctccaaatcagatctacccttagagaataaacatttttctaccgttattttttttcaagtctataatctgagccagtcccaaaggagtgatcaagtttcagaaatgctttcatcttcacaacattttatatatactattatatggggtgaataaagttttaaatccgaaatataaaaaaaaaaaaaaaaaaa

SEQ ID NO: 9 - protein, 2285 aa, Homo sapiens
MAAQVAPAAASSLGNPPPPPPSELKKAEQQQREEAGGEAAAAAAAERGEMKAAAGQESEGPAVGPPQPLGKELQDGAESNGGGGGGGAGSGGGPGAEPDLKNSNGNAGPRPALNNNLTEPPGGGGGGSSDGVGAPPHSAAAALPPPAYGFGQPYGRSPSAVAAAAAAVFHQQHGGQQSPGLAALQSGGGGGLEPYAGPQQNSHDHGFPNHQYNSYYPNRSAYPPPAPAYALSSPRGGTPGSGAAAAAGSKPPPSSSASASSSSSSFAQQRFGAMGGGGPSAAGGGTPQPTATPTLNQLLTSPSSARGYQGYPGGDYSGGPQDGGAGKGPADMASQCWGAAAAAAAAAAASGGAQQRSHHAPMSPGSSGGGGQPLARTPQPSSPMDQMGKMRPQPYGGTNPYSQQQGPPSGPQQGHGYPGQPYGSQTPQRYPMTMQGRAQSAMGGLSYTQQIPPYGQQGPSGYGQQGQTPYYNQQSPHPQQQQPPYSQQPPSQTPHAQPSYQQQPQSQPPQLQSSQPPYSQQPSQPPHQQSPAPYPSQQSTTQQHPQSQPPYSQPQAQSPYQQQQPQQPAPSTLSQQAAYPQPQSQQSQQTAYSQQRFPPPQELSQDSFGSQASSAPSMTSSKGGQEDMNLSLQSRPSSLPDLSGSIDDLPMGTEGALSPGVSTSGISSSQGEQSNPAQSPFSPHTSPHLPGIRGPSPSPVGSPASVAQSRSGPLSPAAVPGNQMPPRPPSGQSDSIMHPSMNQSSIAQDRGYMQRNPQMPQYSSPQPGSALSPRQPSGGQIHTGMGSYQQNSMGSYGPQGGQYGPQGGYPRQPNYNALPNANYPSAGMAGGINPMGAGGQMHGQPGIPPYGTLPPGRMSHASMGNRPYGPNMANMPPQVGSGMCPPPGGMNRKTQETAVAMHVAANSIQNRPPGYPNMNQGGMMGTGPPYGQGINSMAGMINPQGPPYSMGGTMANNSAGMAASPEMMGLGDVKLTPATKMNNKADGTPKTESKSKKSSSSTTTNEKITKLYELGGEPERKMWVDRYLAFTEEKAMGMTNLPAVGRKPLDLYRLYVSVKEIGGLTQVNKNKKWRELATNLNVGTSSSAASSLKKQYIQCLYAFECKIERGEDPPPDIFAAADSKKSQPKIQPPSPAGSGSMQGPQTPQSTSSSMAEGGDLKPPTPASTPHSQIPPLPGMSRSNSVGIQDAFNDGSDSTFQKRNSMTPNPGYQPSMNTSDMMGRMSYEPNKDPYGSMRKAPGSDPFMSSGQGPNGGMGDPYSRAAGPGLGNVAMGPRQHYPYGGPYDRVRTEPGIGPEGNMSTGAPQPNLMPSNPDSGMYSPSRYPPQQQQQQQQRHDSYGNQFSTQGTPSGSPFPSQQTTMYQQQQQNYKRPMDGTYGPPAKRHEGEMYSVPYSTGQGQPQQQQLPPAQPQPASQQQAAQPSPQQDVYNQYGNAYPATATAATERRPAGGPQNQFPFQFGRDRVSAPPGTNAQQNMPPQMMGGPIQASAEVAQQGTMWQGRNDMTYNYANRQSTGSAPQGPAYHGVNRTDEMLHTDQRANHEGSWPSHGTRQPPYGPSAPVPPMTRPPPSNYQPPPSMQNHIPQVSSPAPLPRPMENRTSPSKSPFLHSGMKMQKAGPPVPASHIAPAPVQPPMIRRDITFPPGSVEATQPVLKQRRRLTMKDIGTPEAWRVMMSLKSGLLAESTWALDTINILLYDDNSIMTFNLSQLPGLLELLVEYFRRCLIEIFGILKEYEVGDPGQRTLLDPGRFSKVSSPAPMEGGEEEEELLGPKLEEEEEEEVVENDEEIAFSGKDKPASENSEEKLISKFDKLPVKIVQKNDPFVVDCSDKLGRVQEFDSGLLHWRIGGGDTTEHIQTHFESKTELLPSRPHAPCPPAPRKHVTTAEGTPGTTDQEGPPPDGPPEKRITATMDDMLSTRSSTLTEDGAKSSEAIKESSKFPFGISPAQSHRNIKILEDEPHSKDETPLCTLLDWQDSLAKRCVCVSNTIRSLSFVPGNDFEMSKHPGLLLILGKLILLHHKHPERKQAPLTYEKEEEQDQGVSCNKVEWWWDCLEMLRENTLVTLANISGQLDLSPYPESICLPVLDGLLHWAVCPSAEAQDPFSTLGPNAVLSPQRLVLETLSKLSIQDNNVDLILATPPFSRLEKLYSTMVRFLSDRKNPVCREMAVVLLANLAQGDSLAARAIAVQKGSIGNLLGFLEDSLAATQFQQSQASLLHMQNPPFEPTSVDMMRRAARALLALAKVDENHSEFTLYESRLLDISVSPLMNSLVSQVICDVLFLIGQS

SEQ ID NO: 10 - DNA, 8585 bp, Homo sapiens
cagaaagcggagagtcacagcggggccaggccctggggagcggagcctccaccgcccccctcattcccaggcaagggcttggggggaatgagccgggagagccgggtcccgagcctacagagccgggagcagctgagccgccggcgcctcggccgccgccgccgcctcctcctcctccgccgccgccagcccggagcctgagccggcggggcgggggggagaggagcgagcgcagcgcagcagcggagccccgcgaggcccgcccgggcgggtggggagggcagcccgggggactgggccccggggcggggtgggagggggggagaagacgaagacagggccgggtctctccgcggacgagacagcggggatcatggccgcgcaggtcgcccccgccgccgccagcagcctgggcaacccgccgccgccgccgccctcggagctgaagaaagccgagcagcagcagcgggaggaggcggggggcgaggcggcggcggcggcagcggccgagcgcggggaaatgaaggcagccgccgggcaggaaagcgagggccccgccgtggggccgccgcagccgctgggaaaggagctgcaggacggggccgagagcaatgggggtggcggcggcggcggagccggcagcggcggcgggcccggcgcggagccggacctgaagaactcgaacgggaacgcgggccctaggcccgccctgaacaataacctcacggagccgcccggcggcggcggtggcggcagcagcgatggggtgggggcgcctcctcactcagccgcggccgccttgccgcccccagcctacggcttcgggcaaccctacggccggagcccgtctgccgtcgccgccgccgcggccgccgtcttccaccaacaacatggcggacaacaaagccctggcctggcagcgctgcagagcggcggcggcgggggcctggagccctacgcggggccccagcagaactctcacgaccacggcttccccaaccaccagtacaactcctactaccccaaccgcagcgcctaccccccgcccgccccggcctacgcgctgagctccccgagaggtggcactccgggctccggcgcggcggcggctgccggctccaagccgcctccctcctccagcgcctccgcctcctcgtcgtcttcgtccttcgctcagcagcgcttcggggccatggggggaggcggcccctccgcggccggcgggggaactccccagcccaccgccacccccaccctcaaccaactgctcacgtcgcccagctcggcccggggctaccagggctaccccgggggcgactacagtggcgggccccaggacgggggcgccggcaagggcccggcggacatggcctcgcagtgttggggggctgcggcggcggcagctgcggcggcggccgcctcgggaggggcccaacaaaggagccaccacgcgcccatgagccccgggagcagcggcggcggggggcagccgctcgcccggacccctcagccatccagtccaatggatcagatgggcaagatgagacctcagccatatggcgggactaacccatactcgcagcaacagggacctccgtcaggaccgcagcaaggacatgggtacccagggcagccatacgggtcccagaccccgcagcggtacccgatgaccatgcagggccgggcgcagagtgccatgggcggcctctcttatacacagcagattcctccttatggacaacaaggccccagcgggtatggtcaacagggccagactccatattacaaccagcaaagtcctcaccctcagcagcagcagccaccctactcccagcaaccaccgtcccagacccctcatgcccaaccttcgtatcagcagcagccacagtctcaaccaccacagctccagtcctctcagcctccatactcccagcagccatcccagcctccacatcagcagtccccggctccatacccctcccagcagtcgacgacacagcagcacccccagagccagcccccctactcacagccacaggctcagtctccttaccagcagcagcaacctcagcagccagcaccctcgacgctctcccagcaggctgcgtatcctcagccccagtctcagcagtcccagcaaactgcctattcccagcagcgcttccctccaccgcaggagctatctcaagattcatttgggtctcaggcatcctcagccccctcaatgacctccagtaagggagggcaagaagatatgaacctgagccttcagtcaagaccctccagcttgcctgatctatctggttcaatagatgacctccccatggggacagaaggagctctgagtcctggagtgagcacatcagggatttccagcagccaaggagagcagagtaatccagctcagtctcctttctctcctcatacctcccctcacctgcctggcatccgaggcccttccccgtcccctgttggctctcccgccagtgttgctcagtctcgctcaggaccactctcgcctgctgcagtgccaggcaaccagatgccacctcggccacccagtggccagtcggacagcatcatgcatccttccatgaaccaatcaagcattgcccaagatcgaggttatatgcagaggaacccccagatgccccagtacagttccccccagcccggctcagccttatctccgcgtcagccttccggaggacagatacacacaggcatgggctcctaccagcagaactccatggggagctatggtccccaggggggtcagtatggcccacaaggtggctaccccaggcagccaaactataatgccttgcccaatgccaactaccccagtgcaggcatggctggaggcataaaccccatgggtgccggaggtcaaatgcatggacagcctggcatcccaccttatggcacactccctccagggaggatgagtcacgcctccatgggcaaccggccttatggccctaacatggccaatatgccacctcaggttgggtcagggatgtgtcccccaccagggggcatgaaccggaaaacccaagaaactgctgtcgccatgcatgttgctgccaactctatccaaaacaggccgccaggctaccccaatatgaatcaagggggcatgatgggaactggacctccttatggacaagggattaatagtatggctggcatgatcaaccctcagggacccccatattccatgggtggaaccatggccaacaattctgcagggatggcagccagcccagagatgatgggccttggggatgtaaagttaactccagccaccaaaatgaacaacaaggcagatgggacacccaagacagaatccaaatccaagaaatccagttcttctactacaaccaatgagaagatcaccaagttgtatgagctgggtggtgagcctgagaggaagatgtgggtggaccgttatctggccttcactgaggagaaggccatgggcatgacaaatctgcctgctgtgggtaggaaacctctggacctctatcgcctctatgtgtctgtgaaggagattggtggattgactcaggtcaacaagaacaaaaaatggcgggaacttgcaaccaacctcaatgtgggcacatcaagcagtgctgccagctccttgaaaaagcagtatatccagtgtctctatgcctttgaatgcaagattgaacggggagaagaccctcccccagacatctttgcagctgctgattccaagaagtcccagcccaagatccagcctccctctcctgcgggatcaggatctatgcaggggccccagactccccagtcaaccagcagttccatggcagaaggaggagacttaaagccaccaactccagcatccacaccacacagtcagatccccccattgccaggcatgagcaggagcaattcagttgggatccaggatgcctttaatgatggaagtgactccacattccagaagcggaattccatgactccaaaccctgggtatcagcccagtatgaatacctctgacatgatggggcgcatgtcctatgagccaaataaggatccttatggcagcatgaggaaagctccagggagtgatcccttcatgtcctcagggcagggccccaacggcgggatgggtgacccctacagtcgtgctgccggccctgggctaggaaatgtggcgatgggaccacgacagcactatccctatggaggtccttatgacagagtgaggacggagcctggaatagggcctgagggaaacatgagcactggggccccacagccgaatctcatgccttccaacccagactcggggatgtattctcctagccgctaccccccgcagcagcagcagcagcagcagcaacgacatgattcctatggcaatcagttctccacccaaggcaccccttctggcagccccttccccagccagcagactacaatgtatcaacagcaacagcagaattacaagcggccaatggatggcacatatggccctcctgccaagcggcacgaaggggagatgtacagcgtgccatacagcactgggcaggggcagcctcagcagcagcagttgcccccagcccagccccagcctgccagccagcaacaagctgcccagccttcccctcagcaagatgtatacaaccagtatggcaatgcctatcctgccactgccacagctgctactgagcgccgaccagcaggcggcccccagaaccaatttccattccagtttggccgagaccgtgtctctgcaccccctggcaccaatgcccagcaaaacatgccaccacaaatgatgggcggccccatacaggcatcagctgaggttgctcagcaaggcaccatgtggcaggggcgtaatgacatgacctataattatgccaacaggcagagcacgggctctgccccccagggccccgcctatcatggcgtgaaccgaacagatgaaatgctgcacacagatcagagggccaaccacgaaggctcgtggccttcccatggcacacgccagcccccatatggtccctctgcccctgtgccccccatgacaaggccccctccatctaactaccagcccccaccaagcatgcagaatcacattcctcaggtatccagccctgctcccctgccccggccaatggagaaccgcacctctcctagcaagtctccattcctgcactctgggatgaaaatgcagaaggcaggtcccccagtacctgcctcgcacatagcacctgcccctgtgcagccccccatgattcggcgggatatcaccttcccacctggctctgttgaagccacacagcctgtgttgaagcagaggaggcggctcacaatgaaagacattggaaccccggaggcatggcgggtaatgatgtccctcaagtctggtctcctggcagagagcacatgggcattagataccatcaacatcctgctgtatgatgacaacagcatcatgaccttcaacctcagtcagctcccagggttgctagagctccttgtagaatatttccgacgatgcctgattgagatctttggcattttaaaggagtatgaggtgggtgacccaggacagagaacgctactggatcctgggaggttcagcaaggtgtctagtccagctcccatggagggtggggaagaagaagaagaacttctaggtcctaaactagaagaggaagaagaagaggaagtagttgaaaatgatgaggagatagccttttcaggcaaggacaagccagcttcagagaatagtgaggagaagctgatcagtaagtttgacaagcttccagtaaagatcgtacagaagaatgatccatttgtggtggactgctcagataagcttgggcgtgtgcaggagtttgacagtggcctgctgcactggcggattggtgggggggacaccactgagcatatccagacccacttcgagagcaagacagagctgctgccttcccggcctcacgcaccctgcccaccagcccctcggaagcatgtgacaacagcagagggtacaccagggacaacagaccaggaggggcccccacctgatggacctccagaaaaacggatcacagccactatggatgacatgttgtctactcggtctagcaccttgaccgaggatggagctaagagttcagaggccatcaaggagagcagcaagtttccatttggcattagcccagcacagagccaccggaacatcaagatcctagaggacgaaccccacagtaaggatgagaccccactgtgtacccttctggactggcaggattctcttgccaagcgctgcgtctgtgtgtccaataccattcgaagcctgtcatttgtgccaggcaatgactttgagatgtccaaacacccagggctgctgctcatcctgggcaagctgatcctgctgcaccacaagcacccagaacggaagcaggcaccactaacttatgaaaaggaggaggaacaggaccaaggggtgagctgcaacaaagtggagtggtggtgggactgcttggagatgctccgggaaaacaccttggttacactcgccaacatctcggggcagttggacctatctccataccccgagagcatttgcctgcctgtcctggacggactcctacactgggcagtttgcccttcagctgaagcccaggaccccttttccaccctgggccccaatgccgtcctttccccgcagagactggtcttggaaaccctcagcaaactcagcatccaggacaacaatgtggacctgattctggccacaccccccttcagccgcctggagaagttgtatagcactatggtgcgcttcctcagtgaccgaaagaacccggtgtgccgggagatggctgtggtactgctggccaacctggctcagggggacagcctggcagctcgtgccattgcagtgcagaagggcagtatcggcaacctcctgggcttcctagaggacagccttgccgccacacagttccagcagagccaggccagcctcctccacatgcagaacccaccctttgagccaactagtgtggacatgatgcggcgggctgcccgcgcgctgcttgccttggccaaggtggacgagaaccactcagagtttactctgtacgaatcacggctgttggacatctcggtatcaccgttgatgaactcattggtttcacaagtcatttgtgatgtactgtttttgattggccagtcatgacagccgtgggacacctcccccccccgtgtgtgtgtgcgtgtgtggagaacttagaaactgactgttgccctttatttatgcaaaaccacctcagaatccagtttaccctgtgctgtccagcttctcccttgggaaaaagtctctcctgtttctctctcctccttccacctcccctccctccatcacctcacgcctttctgttccttgtcctcaccttactcccctcaggaccctaccccaccctctttgaaaagacaaagctctgcctacatagaagactttttttattttaaccaaagttactgttgtttacagtgagtttggggaaaaaaaataaaataaaaatggctttcccagtccttgcatcaacgggatgccacatttcataactgtttttaatggtaaaaaaaaaaaaaaaaaatacaaaaaaaaattctgaaggacaaaaaaggtgactgctgaactgtgtgtggtttattgttgtacattcacaatcttgcaggagccaagaagttcgcagttgtgaacagaccctgttcactggagaggcctgtgcagtagagtgtagaccctttcatgtactgtactgtacacctgatactgtaaacatactgtaataataatgtctcacatggaaacagaaaacgctgggtcagcagcaagctgtagtttttaaaaatgtttttagttaaacgttgaggagaaaaaaaaaaaaggcttttcccccaaagtatcatgtgtgaacctacaacaccctgacctctttctctcctccttgattgtatgaataaccctgagatcacctcttagaactggttttaacctttagctgcagcggctacgctgccacgtgtgtatatatatgacgttgtacattgcacatacccttggatccccacagtttggtcctcctcccagctacccctttatagtatgacgagttaacaagttggtgacctgcacaaagcgagacacagctatttaatctcttgccagatatcgcccctcttggtgcgatgctgtacaggtctctgtaaaaagtccttgctgtctcagcagccaatcaacttatagtttatttttttctgggtttttgttttgttttgttttctttctaatcgaggtgtgaaaaagttctaggttcagttgaagttctgatgaagaaacacaattgagattttttcagtgataaaatctgcatatttgtatttcaacaatgtagctaaaacttgatgtaaattcctcctttttttccttttttggcttaatgaatatcatttattcagtatgaaatctttatactatatgttccacgtgttaagaataaatgtacattaaatcttggtaagacttt

SEQ ID NO: 11 - protein, 2068 aa, Homo sapiens
MAAQVAPAAASSLGNPPPPPPSELKKAEQQQREEAGGEAAAAAAAERGEMKAAAGQESEGPAVGPPQPLGKELQDGAESNGGGGGGGAGSGGGPGAEPDLKNSNGNAGPRPALNNNLTEPPGGGGGGSSDGVGAPPHSAAAALPPPAYGFGQPYGRSPSAVAAAAAAVFHQQHGGQQSPGLAALQSGGGGGLEPYAGPQQNSHDHGFPNHQYNSYYPNRSAYPPPAPAYALSSPRGGTPGSGAAAAAGSKPPPSSSASASSSSSSFAQQRFGAMGGGGPSAAGGGTPQPTATPTLNQLLTSPSSARGYQGYPGGDYSGGPQDGGAGKGPADMASQCWGAAAAAAAAAAASGGAQQRSHHAPMSPGSSGGGGQPLARTPQPSSPMDQMGKMRPQPYGGTNPYSQQQGPPSGPQQGHGYPGQPYGSQTPQRYPMTMQGRAQSAMGGLSYTQQIPPYGQQGPSGYGQQGQTPYYNQQSPHPQQQQPPYSQQPPSQTPHAQPSYQQQPQSQPPQLQSSQPPYSQQPSQPPHQQSPAPYPSQQSTTQQHPQSQPPYSQPQAQSPYQQQQPQQPAPSTLSQQAAYPQPQSQQSQQTAYSQQRFPPPQELSQDSFGSQASSAPSMTSSKGGQEDMNLSLQSRPSSLPDLSGSIDDLPMGTEGALSPGVSTSGISSSQGEQSNPAQSPFSPHTSPHLPGIRGPSPSPVGSPASVAQSRSGPLSPAAVPGNQMPPRPPSGQSDSIMHPSMNQSSIAQDRGYMQRNPQMPQYSSPQPGSALSPRQPSGGQIHTGMGSYQQNSMGSYGPQGGQYGPQGGYPRQPNYNALPNANYPSAGMAGGINPMGAGGQMHGQPGIPPYGTLPPGRMSHASMGNRPYGPNMANMPPQVGSGMCPPPGGMNRKTQETAVAMHVAANSIQNRPPGYPNMNQGGMMGTGPPYGQGINSMAGMINPQGPPYSMGGTMANNSAGMAASPEMMGLGDVKLTPATKMNNKADGTPKTESKSKKSSSSTTTNEKITKLYELGGEPERKMWVDRYLAFTEEKAMGMTNLPAVGRKPLDLYRLYVSVKEIGGLTQVNKNKKWRELATNLNVGTSSSAASSLKKQYIQCLYAFECKIERGEDPPPDIFAAADSKKSQPKIQPPSPAGSGSMQGPQTPQSTSSSMAEGGDLKPPTPASTPHSQIPPLPGMSRSNSVGIQDAFNDGSDSTFQKRNSMTPNPGYQPSMNTSDMMGRMSYEPNKDPYGSMRKAPGSDPFMSSGQGPNGGMGDPYSRAAGPGLGNVAMGPRQHYPYGGPYDRVRTEPGIGPEGNMSTGAPQPNLMPSNPDSGMYSPSRYPPQQQQQQQQRHDSYGNQFSTQGTPSGSPFPSQQTTMYQQQQQVSSPAPLPRPMENRTSPSKSPFLHSGMKMQKAGPPVPASHIAPAPVQPPMIRRDITFPPGSVEATQPVLKQRRRLTMKDIGTPEAWRVMMSLKSGLLAESTWALDTINILLYDDNSIMTFNLSQLPGLLELLVEYFRRCLIEIFGILKEYEVGDPGQRTLLDPGRFSKVSSPAPMEGGEEEEELLGPKLEEEEEEEVVENDEEIAFSGKDKPASENSEEKLISKFDKLPVKIVQKNDPFVVDCSDKLGRVQEFDSGLLHWRIGGGDTTEHIQTHFESKTELLPSRPHAPCPPAPRKHVTTAEGTPGTTDQEGPPPDGPPEKRITATMDDMLSTRSSTLTEDGAKSSEAIKESSKFPFGISPAQSHRNIKILEDEPHSKDETPLCTLLDWQDSLAKRCVCVSNTIRSLSFVPGNDFEMSKHPGLLLILGKLILLHHKHPERKQAPLTYEKEEEQDQGVSCNKVEWWWDCLEMLRENTLVTLANISGQLDLSPYPESICLPVLDGLLHWAVCPSAEAQDPFSTLGPNAVLSPQRLVLETLSKLSIQDNNVDLILATPPFSRLEKLYSTMVRFLSDRKNPVCREMAVVLLANLAQGDSLAARAIAVQKGSIGNLLGFLEDSLAATQFQQSQASLLHMQNPPFEPTSVDMMRRAARALLALAKVDENHSEFTLYESRLLDISVSPLMNSLVSQVICDVLFLIGQS

SEQ ID NO: 12 - DNA, 7934 bp, Homo sapiens
cagaaagcggagagtcacagcggggccaggccctggggagcggagcctccaccgcccccctcattcccaggcaagggcttggggggaatgagccgggagagccgggtcccgagcctacagagccgggagcagctgagccgccggcgcctcggccgccgccgccgcctcctcctcctccgccgccgccagcccggagcctgagccggcggggcgggggggagaggagcgagcgcagcgcagcagcggagccccgcgaggcccgcccgggcgggtggggagggcagcccgggggactgggccccggggcggggtgggagggggggagaagacgaagacagggccgggtctctccgcggacgagacagcggggatcatggccgcgcaggtcgcccccgccgccgccagcagcctgggcaacccgccgccgccgccgccctcggagctgaagaaagccgagcagcagcagcgggaggaggcggggggcgaggcggcggcggcggcagcggccgagcgcggggaaatgaaggcagccgccgggcaggaaagcgagggccccgccgtggggccgccgcagccgctgggaaaggagctgcaggacggggccgagagcaatgggggtggcggcggcggcggagccggcagcggcggcgggcccggcgcggagccggacctgaagaactcgaacgggaacgcgggccctaggcccgccctgaacaataacctcacggagccgcccggcggcggcggtggcggcagcagcgatggggtgggggcgcctcctcactcagccgcggccgccttgccgcccccagcctacggcttcgggcaaccctacggccggagcccgtctgccgtcgccgccgccgcggccgccgtcttccaccaacaacatggcggacaacaaagccctggcctggcagcgctgcagagcggcggcggcgggggcctggagccctacgcggggccccagcagaactctcacgaccacggcttccccaaccaccagtacaactcctactaccccaaccgcagcgcctaccccccgcccgccccggcctacgcgctgagctccccgagaggtggcactccgggctccggcgcggcggcggctgccggctccaagccgcctccctcctccagcgcctccgcctcctcgtcgtcttcgtccttcgctcagcagcgcttcggggccatggggggaggcggcccctccgcggccggcgggggaactccccagcccaccgccacccccaccctcaaccaactgctcacgtcgcccagctcggcccggggctaccagggctaccccgggggcgactacagtggcgggccccaggacgggggcgccggcaagggcccggcggacatggcctcgcagtgttggggggctgcggcggcggcagctgcggcggcggccgcctcgggaggggcccaacaaaggagccaccacgcgcccatgagccccgggagcagcggcggcggggggcagccgctcgcccggacccctcagccatccagtccaatggatcagatgggcaagatgagacctcagccatatggcgggactaacccatactcgcagcaacagggacctccgtcaggaccgcagcaaggacatgggtacccagggcagccatacgggtcccagaccccgcagcggtacccgatgaccatgcagggccgggcgcagagtgccatgggcggcctctcttatacacagcagattcctccttatggacaacaaggccccagcgggtatggtcaacagggccagactccatattacaaccagcaaagtcctcaccctcagcagcagcagccaccctactcccagcaaccaccgtcccagacccctcatgcccaaccttcgtatcagcagcagccacagtctcaaccaccacagctccagtcctctcagcctccatactcccagcagccatcccagcctccacatcagcagtccccggctccatacccctcccagcagtcgacgacacagcagcacccccagagccagcccccctactcacagccacaggctcagtctccttaccagcagcagcaacctcagcagccagcaccctcgacgctctcccagcaggctgcgtatcctcagccccagtctcagcagtcccagcaaactgcctattcccagcagcgcttccctccaccgcaggagctatctcaagattcatttgggtctcaggcatcctcagccccctcaatgacctccagtaagggagggcaagaagatatgaacctgagccttcagtcaagaccctccagcttgcctgatctatctggttcaatagatgacctccccatggggacagaaggagctctgagtcctggagtgagcacatcagggatttccagcagccaaggagagcagagtaatccagctcagtctcctttctctcctcatacctcccctcacctgcctggcatccgaggcccttccccgtcccctgttggctctcccgccagtgttgctcagtctcgctcaggaccactctcgcctgctgcagtgccaggcaaccagatgccacctcggccacccagtggccagtcggacagcatcatgcatccttccatgaaccaatcaagcattgcccaagatcgaggttatatgcagaggaacccccagatgccccagtacagttccccccagcccggctcagccttatctccgcgtcagccttccggaggacagatacacacaggcatgggctcctaccagcagaactccatggggagctatggtccccaggggggtcagtatggcccacaaggtggctaccccaggcagccaaactataatgccttgcccaatgccaactaccccagtgcaggcatggctggaggcataaaccccatgggtgccggaggtcaaatgcatggacagcctggcatcccaccttatggcacactccctccagggaggatgagtcacgcctccatgggcaaccggccttatggccctaacatggccaatatgccacctcaggttgggtcagggatgtgtcccccaccagggggcatgaaccggaaaacccaagaaactgctgtcgccatgcatgttgctgccaactctatccaaaacaggccgccaggctaccccaatatgaatcaagggggcatgatgggaactggacctccttatggacaagggattaatagtatggctggcatgatcaaccctcagggacccccatattccatgggtggaaccatggccaacaattctgcagggatggcagccagcccagagatgatgggccttggggatgtaaagttaactccagccaccaaaatgaacaacaaggcagatgggacacccaagacagaatccaaatccaagaaatccagttcttctactacaaccaatgagaagatcaccaagttgtatgagctgggtggtgagcctgagaggaagatgtgggtggaccgttatctggccttcactgaggagaaggccatgggcatgacaaatctgcctgctgtgggtaggaaacctctggacctctatcgcctctatgtgtctgtgaaggagattggtggattgactcaggtcaacaagaacaaaaaatggcgggaacttgcaaccaacctcaatgtgggcacatcaagcagtgctgccagctccttgaaaaagcagtatatccagtgtctctatgcctttgaatgcaagattgaacggggagaagaccctcccccagacatctttgcagctgctgattccaagaagtcccagcccaagatccagcctccctctcctgcgggatcaggatctatgcaggggccccagactccccagtcaaccagcagttccatggcagaaggaggagacttaaagccaccaactccagcatccacaccacacagtcagatccccccattgccaggcatgagcaggagcaattcagttgggatccaggatgcctttaatgatggaagtgactccacattccagaagcggaattccatgactccaaaccctgggtatcagcccagtatgaatacctctgacatgatggggcgcatgtcctatgagccaaataaggatccttatggcagcatgaggaaagctccagggagtgatcccttcatgtcctcagggcagggccccaacggcgggatgggtgacccctacagtcgtgctgccggccctgggctaggaaatgtggcgatgggaccacgacagcactatccctatggaggtccttatgacagagtgaggacggagcctggaatagggcctgagggaaacatgagcactggggccccacagccgaatctcatgccttccaacccagactcggggatgtattctcctagccgctaccccccgcagcagcagcagcagcagcagcaacgacatgattcctatggcaatcagttctccacccaaggcaccccttctggcagccccttccccagccagcagactacaatgtatcaacagcaacagcaggtatccagccctgctcccctgccccggccaatggagaaccgcacctctcctagcaagtctccattcctgcactctgggatgaaaatgcagaaggcaggtcccccagtacctgcctcgcacatagcacctgcccctgtgcagccccccatgattcggcgggatatcaccttcccacctggctctgttgaagccacacagcctgtgttgaagcagaggaggcggctcacaatgaaagacattggaaccccggaggcatggcgggtaatgatgtccctcaagtctggtctcctggcagagagcacatgggcattagataccatcaacatcctgctgtatgatgacaacagcatcatgaccttcaacctcagtcagctcccagggttgctagagctccttgtagaatatttccgacgatgcctgattgagatctttggcattttaaaggagtatgaggtgggtgacccaggacagagaacgctactggatcctgggaggttcagcaaggtgtctagtccagctcccatggagggtggggaagaagaagaagaacttctaggtcctaaactagaagaggaagaagaagaggaagtagttgaaaatgatgaggagatagccttttcaggcaaggacaagccagcttcagagaatagtgaggagaagctgatcagtaagtttgacaagcttccagtaaagatcgtacagaagaatgatccatttgtggtggactgctcagataagcttgggcgtgtgcaggagtttgacagtggcctgctgcactggcggattggtgggggggacaccactgagcatatccagacccacttcgagagcaagacagagctgctgccttcccggcctcacgcaccctgcccaccagcccctcggaagcatgtgacaacagcagagggtacaccagggacaacagaccaggaggggcccccacctgatggacctccagaaaaacggatcacagccactatggatgacatgttgtctactcggtctagcaccttgaccgaggatggagctaagagttcagaggccatcaaggagagcagcaagtttccatttggcattagcccagcacagagccaccggaacatcaagatcctagaggacgaaccccacagtaaggatgagaccccactgtgtacccttctggactggcaggattctcttgccaagcgctgcgtctgtgtgtccaataccattcgaagcctgtcatttgtgccaggcaatgactttgagatgtccaaacacccagggctgctgctcatcctgggcaagctgatcctgctgcaccacaagcacccagaacggaagcaggcaccactaacttatgaaaaggaggaggaacaggaccaaggggtgagctgcaacaaagtggagtggtggtgggactgcttggagatgctccgggaaaacaccttggttacactcgccaacatctcggggcagttggacctatctccataccccgagagcatttgcctgcctgtcctggacggactcctacactgggcagtttgcccttcagctgaagcccaggaccccttttccaccctgggccccaatgccgtcctttccccgcagagactggtcttggaaaccctcagcaaactcagcatccaggacaacaatgtggacctgattctggccacaccccccttcagccgcctggagaagttgtatagcactatggtgcgcttcctcagtgaccgaaagaacccggtgtgccgggagatggctgtggtactgctggccaacctggctcagggggacagcctggcagctcgtgccattgcagtgcagaagggcagtatcggcaacctcctgggcttcctagaggacagccttgccgccacacagttccagcagagccaggccagcctcctccacatgcagaacccaccctttgagccaactagtgtggacatgatgcggcgggctgcccgcgcgctgcttgccttggccaaggtggacgagaaccactcagagtttactctgtacgaatcacggctgttggacatctcggtatcaccgttgatgaactcattggtttcacaagtcatttgtgatgtactgtttttgattggccagtcatgacagccgtgggacacctcccccccccgtgtgtgtgtgcgtgtgtggagaacttagaaactgactgttgccctttatttatgcaaaaccacctcagaatccagtttaccctgtgctgtccagcttctcccttgggaaaaagtctctcctgtttctctctcctccttccacctcccctccctccatcacctcacgcctttctgttccttgtcctcaccttactcccctcaggaccctaccccaccctctttgaaaagacaaagctctgcctacatagaagactttttttattttaaccaaagttactgttgtttacagtgagtttggggaaaaaaaataaaataaaaatggctttcccagtccttgcatcaacgggatgccacatttcataactgtttttaatggtaaaaaaaaaaaaaaaaaatacaaaaaaaaattctgaaggacaaaaaaggtgactgctgaactgtgtgtggtttattgttgtacattcacaatcttgcaggagccaagaagttcgcagttgtgaacagaccctgttcactggagaggcctgtgcagtagagtgtagaccctttcatgtactgtactgtacacctgatactgtaaacatactgtaataataatgtctcacatggaaacagaaaacgctgggtcagcagcaagctgtagtttttaaaaatgtttttagttaaacgttgaggagaaaaaaaaaaaaggcttttcccccaaagtatcatgtgtgaacctacaacaccctgacctctttctctcctccttgattgtatgaataaccctgagatcacctcttagaactggttttaacctttagctgcagcggctacgctgccacgtgtgtatatatatgacgttgtacattgcacatacccttggatccccacagtttggtcctcctcccagctacccctttatagtatgacgagttaacaagttggtgacctgcacaaagcgagacacagctatttaatctcttgccagatatcgcccctcttggtgcgatgctgtacaggtctctgtaaaaagtccttgctgtctcagcagccaatcaacttatagtttatttttttctgggtttttgttttgttttgttttctttctaatcgaggtgtgaaaaagttctaggttcagttgaagttctgatgaagaaacacaattgagattttttcagtgataaaatctgcatatttgtatttcaacaatgtagctaaaacttgatgtaaattcctcctttttttccttttttggcttaatgaatatcatttattcagtatgaaatctttatactatatgttccacgtgttaagaataaatgtacattaaatcttggtaagacttt

(19) United States
(12) Patent Application Publication
(10) Pub. No.: US 2021/0008075 A1
(43) Pub. Date: Jan. 14, 2021

(54) PEDIATRIC DOSING FOR TREATMENT OF CANCER WITH AN EZH2 INHIBITOR

(71) Applicant: Epizyme, Inc., Cambridge, MA (US)

(72) Inventors: Heike KEILHACK, Belmont, MA (US); Sarah K. KNUTSON, Lincoln, MA (US); Nigel WATERS, Belmont, MA (US)

(21) Appl. No.: 16/867,717

(22) Filed: May 6, 2020

Related U.S. Application Data
(63) Continuation of application No. 15/773,757, filed on May 4, 2018.
(60) Provisional application No. 62/252,190, filed on Nov. 6, 2015.

(51) Int. Cl. A61K 31/5377; A61K 31/501; A61K 31/4436; A61K 31/496; A61K 31/4412; A61K 31/444; A61P 35/00; A61K 9/00

(52) U.S. Cl. CPC A61K 31/5377; A61K 31/501; A61K 31/4436; A61K 31/496; A61K 9/0085; A61K 31/444; A61P 35/00; A61K 9/0043; A61K 9/0053; A61K 31/4412

(57) ABSTRACT

The disclosure provides a method of treating a an INI1-deficient tumor in a subject in need thereof comprising administering to the subject a therapeutically-effective amount of an enhancer of a zeste homolog 2 (EZH2) inhibitor. In a preferred embodiment of this method, the subject is pediatric and the EZH2 inhibitor is Tazemetostat.

### RELATED APPLICATIONS

This application is a continuation of U.S. application Ser. No. 15/773,757, filed on May 4, 2018, which is a U.S. National Phase application, filed under 35 U.S.C. § 371, of International Application No. PCT/US2016/060852, filed on Nov. 7, 2016, which claims priority to, and the benefit of U.S. Provisional Application No. 62/252,190 filed Nov. 6, 2015 the contents of each of which are incorporated herein by reference in their entireties. su

### INCORPORATION-BY-REFERENCE OF SEQUENCE LISTING

The contents of the text file named “EPIZ058N01US_ST25.txt,” which was created on Sep. 25, 2018 and is 140 KB in size, are hereby incorporated by reference in their entireties.

### FIELD OF THE DISCLOSURE

The disclosure is directed to the fields of small molecule therapies, cancer, and methods of treating rare cancer types, particularly in pediatric subjects.

### BACKGROUND

There is a long-felt yet unmet need for effective treatments for certain cancers caused by genetic alterations or loss of function of subunits of the SWI/SNF chromatin remodeling complex that result in EZH2-dependent oncogenesis.

### SUMMARY

Some aspects of this disclosure provide methods, strategies, and dosage schedules for inhibiting EZH2 in a subject, e.g., in a human pediatric patient, by administering a therapeutically-effective amount of an enhancer of a zeste homolog 2 (EZH2) inhibitor to the subject. The methods, strategies, and dosage schedules provided herein are useful, for example, for treating cancer in pediatric patients.

Some aspects of this disclosure provide a method of treating a cancer, e.g., an INI1-deficient tumor, in a subject in need thereof comprising administering to the subject a therapeutically-effective amount of an enhancer of a zeste homolog 2 (EZH2) inhibitor. Methods of treating cancer, e.g., INI1-deficient tumors, provided herein may comprise preventing and/or inhibiting proliferation of a malignant cell, e.g., an INI1-deficient cell, or cell population.

In certain embodiments of the methods of the disclosure, the EZH2 inhibitor comprises

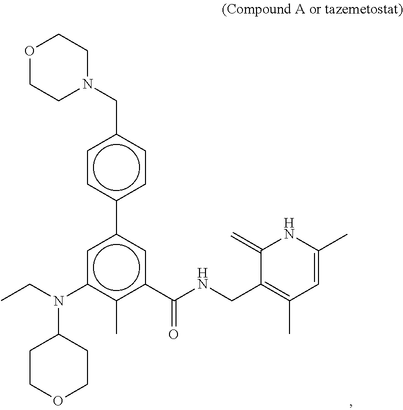

or a pharmaceutically-acceptable salt thereof.

In certain embodiments of the methods of the disclosure, the EZH2 inhibitor comprises

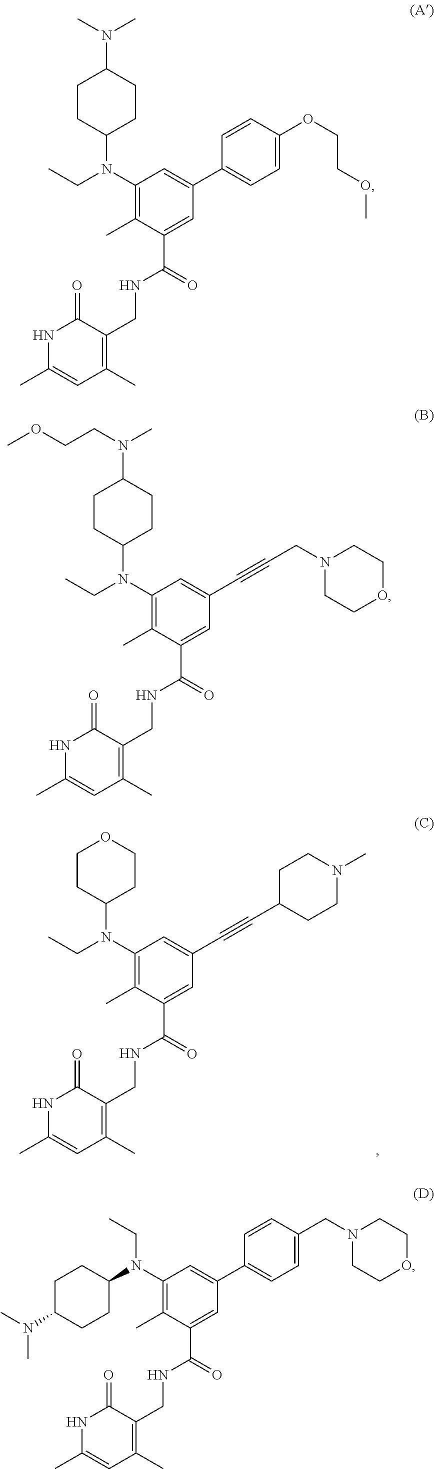

a stereoisomer, a pharmaceutically acceptable salt and/or a solvate thereof.

In certain embodiments of the methods of the disclosure, the EZH2 inhibitor comprises

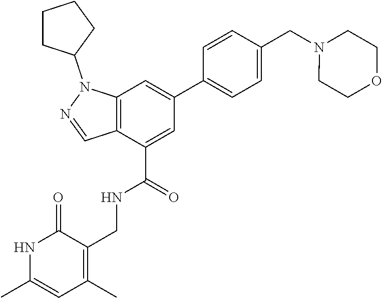 [SMILES: Cc1cc(C)c(CNC(=O)c2cc(-c3ccc(CN4CCOCC4)cc3)cc3c2cnn3C2CCCC2)c(=O)[nH]1]

or a pharmaceutically acceptable salt thereof.

In certain embodiments of the methods of the disclosure, the EZH2 inhibitor comprises

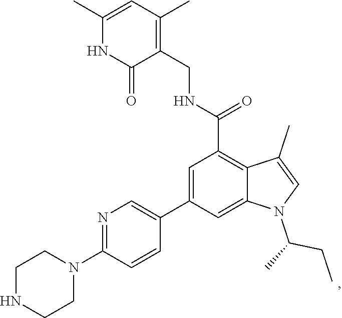 [SMILES: CC[C@H](C)n1cc(C)c2c(C(=O)NCc3c(C)cc(C)[nH]c3=O)cc(-c3ccc(N4CCNCC4)nc3)cc21]

a stereoisomer, a pharmaceutically acceptable salt and/or a solvate thereof.

In certain embodiments of the methods of the disclosure, the EZH2 inhibitor comprises

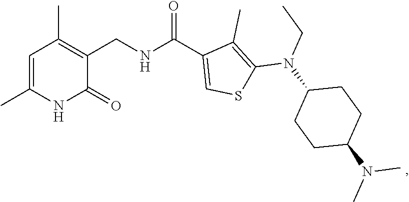

a stereoisomer, a pharmaceutically acceptable salt and/or a solvate thereof.

In certain embodiments of the methods of the disclosure, the EZH2 inhibitor comprises

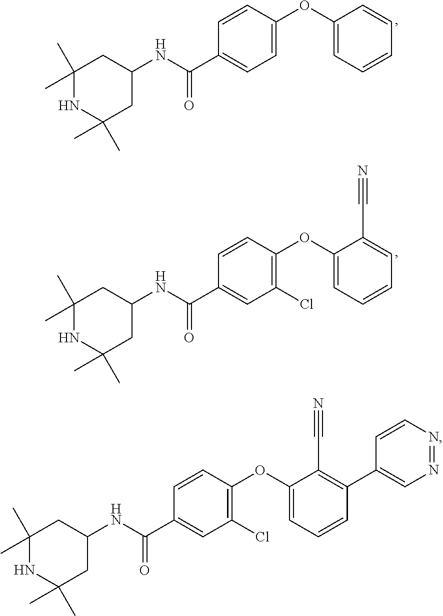 [SMILES: CC1(C)CC(NC(=O)c2ccc(Oc3ccccc3)cc2)CC(C)(C)N1 ; CC1(C)CC(NC(=O)c2ccc(Oc3ccccc3C#N)c(Cl)c2)CC(C)(C)N1 ; CC1(C)CC(NC(=O)c2ccc(Oc3cccc(-c4ccnnc4)c3C#N)c(Cl)c2)CC(C)(C)N1]

a stereoisomer, a pharmaceutically acceptable salt and/or a solvate thereof.

EZH2 inhibitors of the disclosure may be administered orally. For example, the EZH2 inhibitor may be formulated as an oral tablet or suspension.

EZH2 inhibitors of the disclosure may be formulated for administration to cerebral spinal fluid (C SF) by any route. Exemplary routes of administration to the CSF include, but are not limited to, an intraspinal, an intracranial, an intrathecal or an intranasal route.

In certain embodiments, including, but not limited to, those embodiments wherein the EZH2 inhibitor is formulated as an oral tablet, or as a suspension or solution, EZH2 inhibitors of the disclosure may be administered at a dose of between 10 mg/kg/day and 1600 mg/kg/day. EZH2 inhibitors of the disclosure may be administered at a dose of about 100, 200, 400, 800, or 1600 mg. EZH2 inhibitors of the disclosure may be administered at a dose of about 800 mg. EZH2 inhibitors of the disclosure may be administered once or twice per day (BID). In some embodiments, EZH2 inhibitors of the disclosure may be administered at a dose of between 10 mg/kg/day and 1600 mg/kg/day BID. For example, in some embodiments, EZH2 inhibitors of the disclosure may be administered at a dose of 800 mg BID.

In some embodiments, including, but not limited to, those embodiments wherein the EZH2 inhibitor is formulated as an oral tablet, suspension, or solution, and/or formulated for administration to the CSF by any route, the EZH2 inhibitor may be administered at a dose of between 10 mg/kg/day and 1600 mg/kg/day, e.g., at a dose of 10 mg/kg/day, 20 mg/kg/day, 25 mg/kg/day, 30 mg/kg/day, 40 mg/kg/day, 50 mg/kg/day, 60 mg/kg/day, 70 mg/kg/day, 75 mg/kg/day, 80 mg/kg/day, 90 mg/kg/day, 100 mg/kg/day, 200 mg/kg/day, 250 mg/kg/day, 300 mg/kg/day, 400 mg/kg/day, 500 mg/kg/day, 600 mg/kg/day, 700 mg/kg/day, 750 mg/kg/day, 800 mg/kg/day, 900 mg/kg/day, 1000 mg/kg/day, 1100 mg/kg/day, 1200 mg/kg/day, 1250 mg/kg/day, 1300 mg/kg/day, 1400 mg/kg/day, 1500 mg/kg/day, or 1600 mg/kg/day. For example, EZH2 inhibitors of the disclosure may be administered at a dose of between 10 mg/kg/day and 1600 mg/kg/day BID. For example, EZH2 inhibitors of the disclosure may be administered at a dose of 800 mg BID.

In some embodiments, including, but not limited to, those embodiments wherein the EZH2 inhibitor is formulated as an oral tablet, suspension, or solution, and/or formulated for administration to the CSF by any route, the EZH2 inhibitor may be administered at a dose of between 10 mg/m²/day and 1200 mg/m²/day, e.g., at a dose of 10 mg/m²/day, 20 mg/m²/day, 25 mg/m²/day, 30 mg/m²/day, 40 mg/m²/day, 50 mg/m²/day, 60 mg/m²/day, 70 mg/m²/day, 75 mg/m²/day, 80 mg/m²/day, 90 mg/m²/day, 100 mg/m²/day, 110 mg/m²/day, 120 mg/m²/day, 125 mg/m²/day, 130 mg/m²/day, 140 mg/m²/day, 150 mg/m²/day, 160 mg/m²/day, 170 mg/m²/day, 175 mg/m²/day, 180 mg/m²/day, 190 mg/m²/day, 200 mg/m²/day, 210 mg/m²/day, 220 mg/m²/day, 225 mg/m²/day, 230 mg/m²/day, 240 mg/m²/day, 250 mg/m²/day, 260 mg/m²/day, 270 mg/m²/day, 275 mg/m²/day, 280 mg/m²/day, 290 mg/m²/day, 300 mg/m²/day, 310 mg/m²/day, 320 mg/m²/day, 325 mg/m²/day, 330 mg/m²/day, 340 mg/m²/day, 350 mg/m²/day, 360 mg/m²/day, 370 mg/m²/day, 375 mg/m²/day, 380 mg/m²/day, 390 mg/m²/day, 400 mg/m²/day, 410 mg/m²/day, 420 mg/m²/day, 425 mg/m²/day, 430 mg/m²/day, 440 mg/m²/day, 450 mg/m²/day, 460 mg/m²/day, 470 mg/m²/day, 475 mg/m²/day, 480 mg/m²/day, 490 mg/m²/day, 500 mg/m²/day, 525 mg/m²/day, 550 mg/m²/day, 575 mg/m²/day, 600 mg/m²/day, 625 mg/m²/day, 650 mg/m²/day, 675 mg/m²/day, 700 mg/m²/day, 750 mg/m²/day, 800 mg/m²/day, 850 mg/m²/day, 900 mg/m²/day, or 1000 mg/m2/day. In some embodiments, including, but not limited to, those embodiments wherein the EZH2 inhibitor is formulated as an oral tablet, suspension, or solution, and/or formulated for administration to the CSF by any route, the EZH2 inhibitor may be administered at a dose of between 10 mg/m²/day and 1200 mg/m²/day, e.g., between 100 and 300 mg/m²/day, between 200 and 300 mg/m²/day, between 200 and 400 mg/m²/day, between 250 and 500 mg/m²/day, between 150 and 400 mg/m²/day, between 150 and 300 mg/m²/day, between 300 and 600 mg/m²/day, between 350 and 400 mg/m²/day, between 350 and 700 mg/m²/day, or between 400 and 1200 mg/m²/day. For example, EZH2 inhibitors of the disclosure may be administered at a dose of between 10 mg/m²/day and 1200 mg/m²/day BID. For example, EZH2 inhibitors of the disclosure may be administered at a dose of 100, 120, 140, 150, 160, 200, 240, 250, 260, 300, 320, 350, 380, 400, or 600 mg/m2 BID.

In certain embodiments, including, but not limited to, those embodiments wherein the EZH2 inhibitor is formulated as an oral tablet, or as a suspension or solution and/or formulated for administration to the CSF by any route, EZH2 inhibitors of the disclosure may be administered at a dose of 50%, 60%, 70%, 80%, 90%, or any percentage in between of a value of an area under the curve (AUC) of a steady state plasma and/or CSF concentration (AUC_{SS}) of an EZH2 inhibitor, wherein the AUC_{SS} is determined following administration of the EZH2 inhibitor to an adult subject at a dose of between 10 mg/kg/day and 1600 mg/kg/day BID. In certain embodiments of the methods of the disclosure, including, but not limited to, those embodiments wherein the EZH2 inhibitor is formulated as an oral suspension and/or formulated to administration to the CSF by any route, EZH2 inhibitors of the disclosure may be administered at a dose of between 230 mg/m² and 600 mg/m², inclusive of the endpoints. EZH2 inhibitors of the disclosure may be administered at a dose of between 300 mg/m² and 600 mg/m². EZH2 inhibitors of the disclosure may be administered at a dose of between 230 mg/m² and 305 mg/m², inclusive of the endpoints. EZH2 inhibitors of the disclosure may be administered at a dose of 240 mg/m². EZH2 inhibitors of the disclosure may be administered at a dose of 300 mg/m². EZH2 inhibitors of the disclosure may be administered once or twice per day (BID). For example, EZH2 inhibitors of the disclosure may be administered at a dose of between 230 mg/m² and 600 mg/m² BID, inclusive of the endpoints.

For example, an EZH2 inhibitor of the disclosure may be administered at a dose of about 60% of the area under the curve (AUC) at steady state (AUC_{SS}) following administration of 1600 mg twice a day to an adult subject. Accordingly, an EZH2 inhibitor of the disclosure administered at a dose of about 60% of the area under the curve (AUC) at steady state (AUC_{SS}) following administration of 1600 mg twice a day to an adult subject, is administered at a dose of about 600 mg/m² per day or at least 600 mg/m² per day. In certain aspects of this example, the subject treated with the EZH2 inhibitor is a pediatric subject.

For example, an EZH2 inhibitor of the disclosure may be administered at a dose of about 80% of the area under the curve (AUC) at steady state (AUC_{S}) following administration of 800 mg twice a day to an adult subject. Accordingly, an EZH2 inhibitor of the disclosure administered at a dose of about 80% of the area under the curve (AUC) at steady state (AUC_{SS}) following administration of 800 mg twice a day to an adult subject, is administered at a dose of about 390 mg/m² per day or at least 390 mg/m² per day. In certain aspects of this example, the subject treated with the EZH2 inhibitor is a pediatric subject.

In some embodiment, the subject may be a pediatric subject. In some embodiments, a pediatric subject of the disclosure is between 6 months and 21 years of age, inclusive of the endpoints. For example, in some embodiments, a pediatric subject of the disclosure is between 1 year and 18 years of age, inclusive of the endpoints; 10 years of age or less; 5 years of age or less; between 6 months and 1 year of age, inclusive of the endpoints; between 1 year and 2 years of age, inclusive of the endpoints; between 2 years and 6 years of age, inclusive of the endpoints; between 6 years and 12 years of age, inclusive of the endpoints; or between 12 years and 18 years of age, inclusive of the endpoints. In some embodiments, a pediatric subject is about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, or about 21 years of age. In some embodiments, a pediatric subject is at least 1, 2, 3, 4, 5, 6, 7, 8, 9, 10, or 12 years of age, and not more than 5, 6, 7, 8, 9, 10, 11, 12, 13, 14 ,15, 16, 17, 18, 19, 20, or 21 years of age, wherein every possible age range that can be formed with these values (e.g., at least 4 and not older than 12 years, or at least 10 and not older than 18 years, to provide two non-limiting examples) is embraced by the present disclosure.

In some embodiments, the disclosure provides a method of treating cancer, e.g., an INI1-deficient tumor, in a subject in need thereof comprising administering to the subject a therapeutically-effective amount of tazemetostat, wherein the therapeutically effective amount is at least 300 mg/m² twice per day (BID), and wherein the subject is a pediatric subject, e.g., a subject between 6 months and 21 years of age, inclusive of the endpoints.

### BRIEF DESCRIPTION OF THE DRAWINGS

FIGS. 1A and 1B are a series of Western blot analyses of cell lines with wild type (RD and SJCRH30) and mutant SNF5.

FIGS. 2A-2E are a series of graphs establishing that SNF5 mutant cell lines A204 (FIG. 2C), G401 (FIG. 2D) and G402 (FIG. 2E) selectively respond to EZH2 compound (Compound D) compared to wild type cell lines RD (FIG. 2A) and SJCRH30 (FIG. 2B).

FIGS. 3A-3D are a series of bar graphs showing that G401 SNF mutant cell line is responding to Compound D after 7 days in soft agar compared to wild type cells RD. FIG. 3A shows cell line RD (5,000 cells/well). FIG. 3B shows G401 cells (5,000 cells/well). FIG. 3C shows G401 cells in 2D growth. FIG. 3D shows G401 cells (10,000 cells/well).

FIGS. 4A-4D are four graphs showing that G401 SNF5 mutant cell line is sensitive to Compound A in vitro. Wild type cell line SJCRH30 (FIG. 4A) and RD (FIG. 4C) and SNF5 mutant cell line G401 (FIG. 4B) and A204 (FIG. 4D) were pretreated for 7 days with indicated concentrations of Compound A and replated on day 0. Cell viability was determined by CellTiter-Glo® Luminescent Cell Viability Assay.

FIGS. 5A-5E are a series of graphs showing durable regressions in G401 xenografts (malignant rhabdoid tumor model) with Compound A treatment. (FIG. 5A) Tumor regressions induced by Compound A at the indicated doses. (FIG. 5B) Tumor regressions induced by twice daily administration of Compound A at the indicated doses. Data represent the mean values±SEM (n=8). Compound administration was stopped on day 28. (FIG. 5C) EZH2 target inhibition in G401 xenograft tumor tissue collected from a parallel cohort of mice on day 21. Each point shows the ratio of H3K27Me3 to total H3. Horizontal lines represent group mean values. BLLQ=below lower limit of quantification. (FIG. 5D, FIG. 5E) Immunohistochemical staining of tumor histone methylation of tumor samples from the vehicle treated (FIG. 5D) and Compound A treated (FIG. 5E) (at 125 mg/kg) mice.

FIG. 6 is a graph showing the locations of ATRX mutations identified in SCLC cell lines.

FIG. 7A is a graph showing that LNCAP prostate cancer cells display dose-dependent cell growth inhibition with Compound D treatment in vitro.

FIG. 7B is a graph showing IC50 value of Compound D at day 11 and day 14 for WSU-DLCL2 and LNCAP cells.

FIGS. 8A-8C are three graphs establishing that ATRX mutant SCLC lines NCI-H446 (FIG. 8A), SW1271 (FIG. 8B) and NCI-H841 (FIG. 8C) are responding to Compound D.

FIGS. 9A-9C are three microscopy images showing that SCLC line NCI-H841 changes morphology after treatment with vehicle (FIG. 9A) or Compound D at concentration of 4.1E-02 uM (FIG. 9B) or 3.3 uM (FIG. 9C).

FIGS. 10A-10C are a series of graphs showing effects of Compound A on cellular global histone methylation and cell viability. (FIG. 10A) Chemical structure of Compound A (or tazemetostat). (FIG. 10B) Concentration-dependent inhibition of cellular H3K27Me3 levels in G401 and RD cells. (FIG. 10C) Selective inhibition of proliferation of SMARCB1-deleted G401 cells by Compound A in vitro (measured by ATP content). G401 (FIGS. 10C and 10D) and RD cells (FIGS. 10E and 10F) were re-plated at the original seeding densities on day 7. Each point represents the mean for each concentration (n=3).

FIGS. 11A and 11B are a series of graphs showing biochemical mechanism of action studies. The IC50 value of Compound A increases with increasing SAM concentration (FIG. 11A) and is minimally affected by increasing oligonucleosome concentration (FIG. 11B), indicating SAM-competitive and nucleosome-noncompetitive mechanism of action.

FIGS. 12A and 12B are a series of panels demonstrating verification of SMARCB1 and EZH2 expression in cell lines and specificity of Compound A for inhibition of cellular histone methylation. (FIG. 12A) Cell lysates were analyzed by immunoblot with antibodies specific to SMARCB1, EZH2 and Actin (loading control). (FIG. 12B) Selective inhibition of cellular H3K27 methylation in G401 and RD cells. Cells were incubated with Compound A for 4 days, and acid-extracted histones were analyzed by immunoblot.

FIGS. 13A and 13B are a series of bar graphs demonstrating that Compound A induces G₁ arrest and apoptosis in SMARCB1-deleted MRT cells. Cell cycle analysis (by flow cytometry) and determination of apoptosis (by TUNEL assay) in RD (FIG. 13A) or G401 cells (FIG. 13B) during incubation with either vehicle or 1 μM Compound A for up to 14 days. G₁ arrest was observed as of day 7 and apoptosis was induced as of day 11. Data are represented as mean values±SEM (n=2). The DMSO control values shown are the average±SEM from each time point. Cells were split and re-plated on days 4, 7 and 11 at the original seeding density.

FIGS. 14A-14C are a series of graphs showing that Compound A induces changes in expression of SMARCB1 regulated genes and cell morphology. (FIG. 14A) Basal expression of SMARCB1 regulated genes in G401 SMARCB1-deleted cells, relative to RD control cells (measured by qPCR, n=2). (FIGS. 14B-14L) G401 and RD cells were incubated with either DMSO or 1 μM Compound A for 2, 4 and 7 days. Gene expression was determined by qPCR (n=2) and is expressed relative to the DMSO control of each time point. FIGS. 14B-14K correspond to genes GLI1, PTCh1, DOCK4, CD133, PTPRK, BIN1, CDKN1A, CDKN2A, EZH2, and MYC, respectively. (FIG. 14L) G402 cells were incubated with either DMSO (left panel) or 1 μM Compound A (right panel) for 14 days. Cells were split and re-plated to the original seeding density on day 7.

FIGS. 15A-15D are series of graphs demonstrating body weights, tumor regressions and plasma levels in G401 xenograft bearing mice treated with Compound A. (FIG. 15A) Body weights were determined twice a week for animals treated with Compound A on a BID schedule for 28 days. Data are presented as mean values±SEM (n=16 until day 21, n=8 from day 22 to 60). (FIG. 15B) Tumor regressions induced by twice daily (BID) administration of Compound A for 21 days at the indicated doses (mean values±SEM, n=16). *p<0.05, **p<0.01, repeated measures ANOVA, Dunnett's post-test vs. vehicle. (FIG. 15C) Tumor weights of 8 mice euthanized on day 21. ****p<0.0001, Fisher's exact test. (FIG. 15D) Plasma was collected 5 min before and 3 h after dosing of Compound A on day 21, and compound levels were measured by LC-MS/MS. Animals were euthanized, and tumors were collected 3 h after dosing on day 21. Tumor homogenates were generated and subjected to LC-MS/MS analysis to determine Compound A concentrations. Note that tumor compound levels could not be determined from all animals especially in the higher dose groups because the xenografts were too small on day 21. Dots represent values for the individual animals; horizontal lines represent group mean values.

FIGS. 16A-16C are a series of graphs showing that Compound A eradicates SMARCB1-deleted MRT xenografts in SCID mice. (FIG. 16A) Tumor regressions induced by twice daily (BID) administration of Compound A for 28 days at the indicated doses. Compound administration was stopped on day 28 and tumors were allowed to re-grow until they reached 2000 mm³ (data shown as mean values±SEM, n=8). (FIG. 16B) EZH2 target inhibition in G401 xenograft tumor tissue collected from mice euthanized on day 21. Each point shows the ratio of H3K27Me3 to total H3, measured by ELISA. Horizontal lines represent group mean values; grey symbols are values outside of the ELISA standard curve. (FIGS. 16C-16F) Change in gene expression in G401 xenograft tumor tissue collected from mice treated with Compound A for 21 days. FIGS. 16C-16F correspond to genes CD133, PTPRK, DOCK4, and GLI1, respectively. Data are presented as fold change compared to vehicle±SEM (n=6, n=4 for 500 mg/kg group). *p<0.05, **p<0.01, ****p<0.0001, vs. vehicle, Fisher's exact test.

FIG. 17 is a schematic diagram depicting epigenetic control of gene expression. Combinations of histone modifications encode information that governs coordinated activation or repression of genetic programs as well as developmental cell identity and fate decisions.

FIG. 18 is a graph showing that EZH2 is over expressed and associated with chromosome 7 amplification in medulloblastoma. Solid bars indicate a balanced chromosome 7 whereas hatched bars indicate a chromosome 7 gain.

FIG. 19 is a schematic diagram depicting control of histone lysine methylation by EZH2 and MLL.

FIG. 20A is a graph showing the probability of overall survival (OS) as a function of time since diagnosis (in months) with medulloblastoma. Histone lysine methylation is altered in medulloblastoma. H3K27me3 abundance is increased in medulloblastoma cells compared to control cells.

FIG. 20B is a graph showing the probability of overall survival (OS) as a function of time since diagnosis (in months) with medulloblastoma. Histone lysine methylation is altered in medulloblastoma. H3K27me3 abundance is increased in medulloblastoma cells compared to control cells.

FIG. 21A is a series of photographs and a graph showing the abundances of H3K4me3 and H3K27Me3 in medulloblastoma cells. The data demonstrate deregulation of the histone code in medulloblastoma.

FIG. 21B is a graph depicting the probability of overall survival as a function of time since diagnosis (in months) for medulloblastoma subjects having deregulated histone methylation at H3K4me3 and/or H3K27Me3.

FIG. 22A is a graph demonstrating that inhibition of EZH2 by a short-hairpin EZH2 (shEZH2) construct suppresses medulloblastoma cell growth (growth of the DAOY medulloblastoma cell line) compared to a negative-control construct.

FIG. 22B a series of photographs and a graph demonstrating that inhibition of EZH2 by a short-hairpin EZH2 (shEZH2) construct suppresses medulloblastoma cell growth (growth of the DAOY medulloblastoma cell line) compared to a negative-control construct and/or the empty pSIF vector control.

FIG. 23A is a schematic diagram depicting the mechanism by which INI1 loss creates an oncogenic dependency on EZH2 in tumors.

FIG. 23B is a graph showing the percent of tumor-free survival of INI1 deficient mice as a function of time (days) when EZH2 is knocked out. EZH2 knockout reverses oncogenesis induced by INI1 loss.

FIG. 24A is a series of photographs showing control or EZH2 inhibitor-treated (DNZep-treated) atypical teratoid rhabdoid tumors (ATRTs) at 1, 3, 5, and 7 days post-treatment. Inhibition of EZH2 suppresses ATRT cell self-renewal.

FIG. 24B is a graph quantifying the results of FIG. 24A.

FIG. 24C is a graph quantifying the results of FIG. 24A.

FIG. 24D is a series of photographs showing control or EZH2 inhibitor-treated (DNZep-treated) atypical teratoid rhabdoid tumors (ATRTs) at 3, 5, 8 and 10 days post-treatment. Inhibition of EZH2 suppresses ATRT cell self-renewal.

FIG. 24E is a graph quantifying the results of FIG. 24D.

FIG. 25A is a pair of graphs showing a surviving fraction of untreated or DZNEP-treated ATRT cells (from a BT-16 ATRT cell line) exposed to 2Gy radiation. Inhibition of EZH2 radio-sensitizes ATRT.

FIG. 25B is a pair of graphs showing a surviving fraction of untreated or DZNEP-treated ATRT cells (from a UPN737 ATRT cell line, “737”) exposed to 2Gy radiation. Inhibition of EZH2 radio-sensitizes ATRT.

FIG. 26A is a graph showing the concentration of medulloblastoma cells (total cells per milliliter) as a function of time (days) following treatment with GSK-126, a small molecule inhibitor of EZH2. Small molecule inhibitors of EZH2 decrease medulloblastoma cell growth.

FIG. 26B is a graph showing the concentration of medulloblastoma cells (total cells per milliliter) as a function of time (days) following treatment with UNC 1999, a small molecule inhibitor of EZH2. Small molecule inhibitors of EZH2 decrease medulloblastoma cell growth.

FIG. 26C is a graph showing the concentration of medulloblastoma cells (total cells per milliliter) as a function of time (days) following treatment with tazemetostat (EPZ 6438), a small molecule inhibitor of EZH2. Small molecule inhibitors of EZH2 decrease medulloblastoma cell growth.

FIG. 26D is a graph showing the concentration of medulloblastoma cells (total cells per milliliter) as a function of time (days) following treatment with GSK-126, UNC 1999, and tazemetostat (EPZ 6438). Tazemetostat has the greatest effect on medulloblastoma cell growth of the small molecule inhibitors tested.

FIG. 27A is a chemical structure diagram of tazemetostat.

FIG. 27B is a pair of schematic diagrams depicting the relative selectivity of tazemetostat for EZH2.

FIG. 28A is a schematic diagram depicting the process by which primary medulloblastoma cell growth is evaluated ex vivo.

FIG. 28B is a graph depicting the relative abundances (percent of cells) of untreated or tazemetostat (EPZ 6438)-treated primary medulloblastoma cells in various cell cycle stages (sub Go/G1, Go/G1, S, or G2/M). A slice culture of medulloblastoma was freshly isolated from a 5 year old subject. The slice culture was treated with tazemetostat for 4 days before being disaggregated and analyzed by flow cytometry. Tazemetostat treatment decreases primary medulloblastoma cell growth ex vivo.

FIG. 28C is a graph depicting BrdU expression of the cells analyzed in FIG. 28B. Tazemetostat treatment decreases primary medulloblastoma cell growth ex vivo.

FIG. 29A is a graph depicting percent survival of vehicle or tazemetostat (EPZ 6438)-treated ATRT cells in vivo as a function of time (days) post-treatment. Tazemetostat decreases ATRT in vivo.

FIG. 29B is a photograph of a Western blot showing the relative amounts of H2K27me3 and H3 in vehicle or tazemetostat (EPZ 6438)-treated ATRT cells from FIG. 29A.

FIG. 30 is a schematic illustrating the generalized layout of a physiologically-based pharmacokinetic (PBPK) model.

FIG. 31 is a scheme illustrating the modeling and simulation for pediatric starting dose selection in early clinical development.

FIG. 32 is a series of graphs showing that the model fit for interim adult PK data at steady-state (Day 15, n=24) showed a good fit for each dose group. Symbols represent observed data from individuals (+/−SD, n=3 or 6 per dose) and the solid line represents the mean profile predicted from the PBPK model in Gastroplus'. The dotted grey lines represent the 90% CI. Tazemetostat PK data from patients enrolled in the dose escalation cohorts of a phase 1 clinical study were previously presented by Ribrag et al., Blood (2015) 126:473, the content of which is incorporated herein by reference in its entirety.

FIG. 33 is a pair of graphs showing the predicted mean total steady state plasma concentration-time profiles of tazemetostat administered as a 240 mg/m² BID or 300 mg/m² BID oral dose across the age ranges and mean measured total steady-state plasma concentration-time profile of tazemetostat administered as a 390 mg/m² (800 mg) or 780 mg/m² (1600 mg) BID oral dose in adults (n=6 per dose). The adult model was used to predict the PK profile in pediatric populations by accounting for age-dependent physiological differences, such as ontogeny of the GI tract and other organs, blood flows, P450 expression, plasma protein binding and hematocrit.

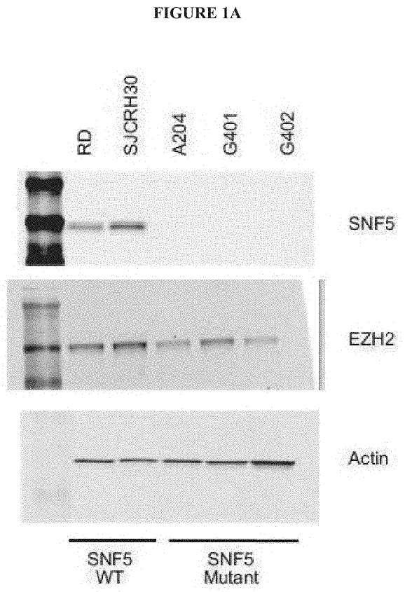

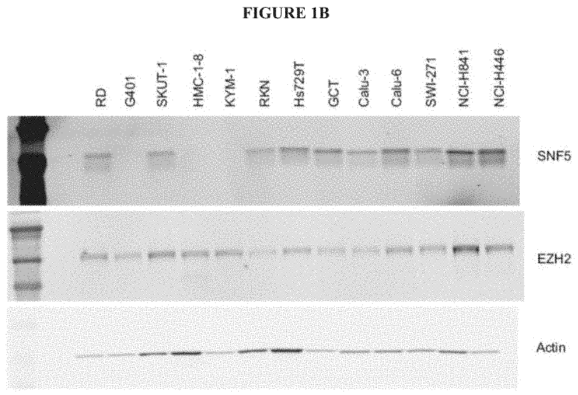

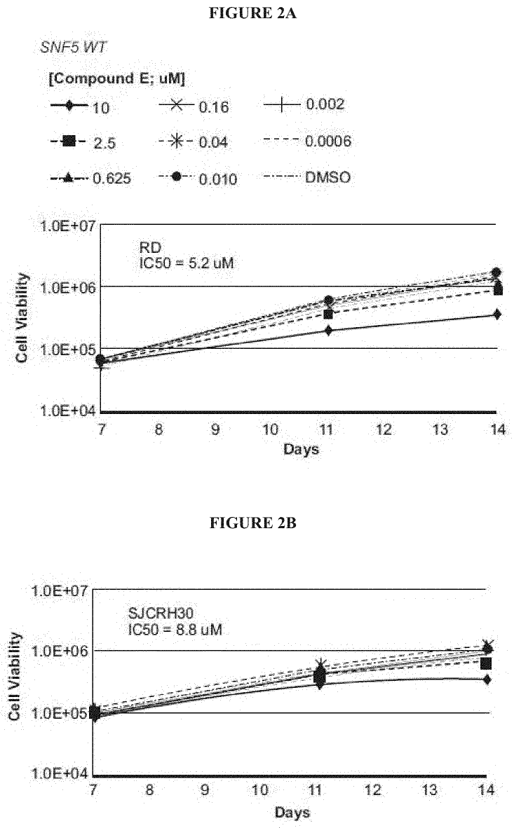

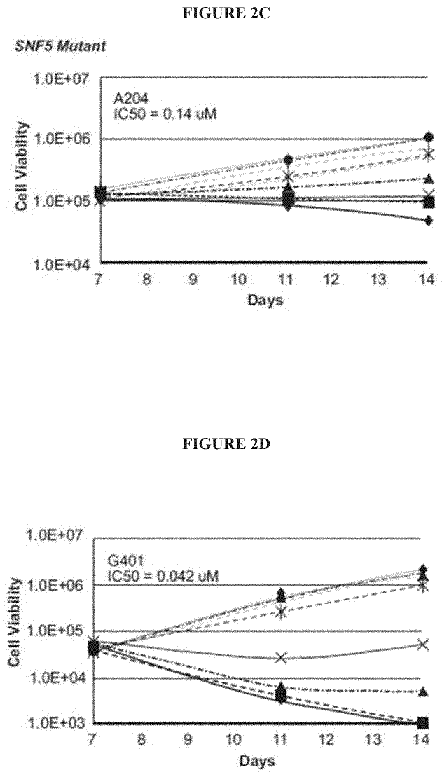

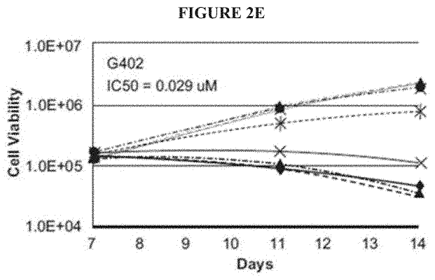

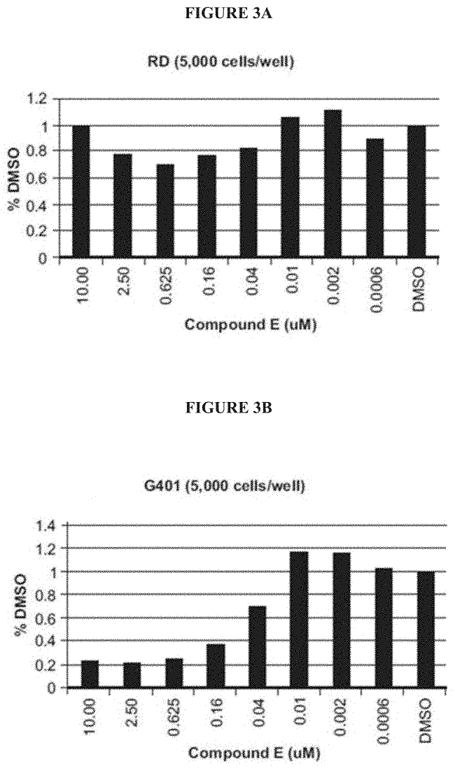

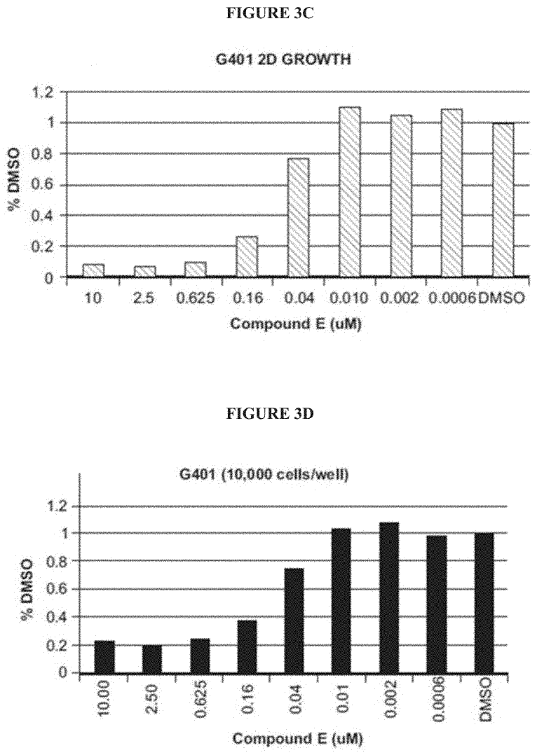

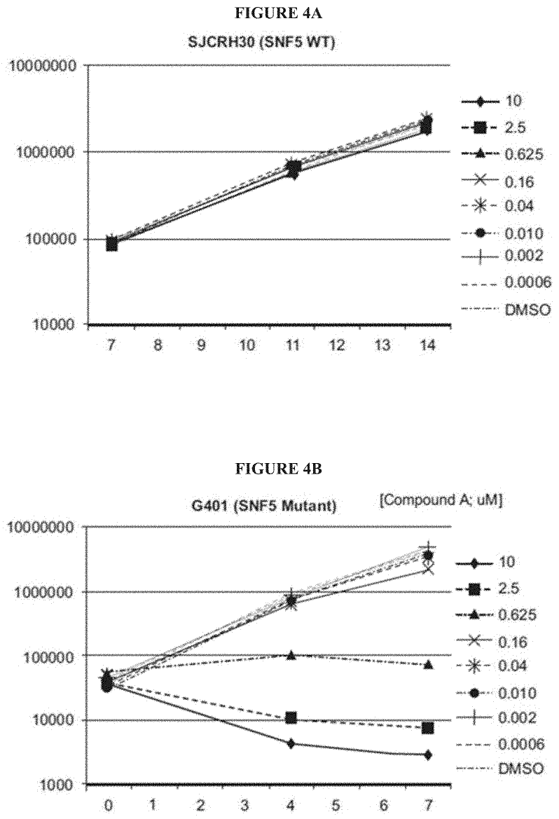

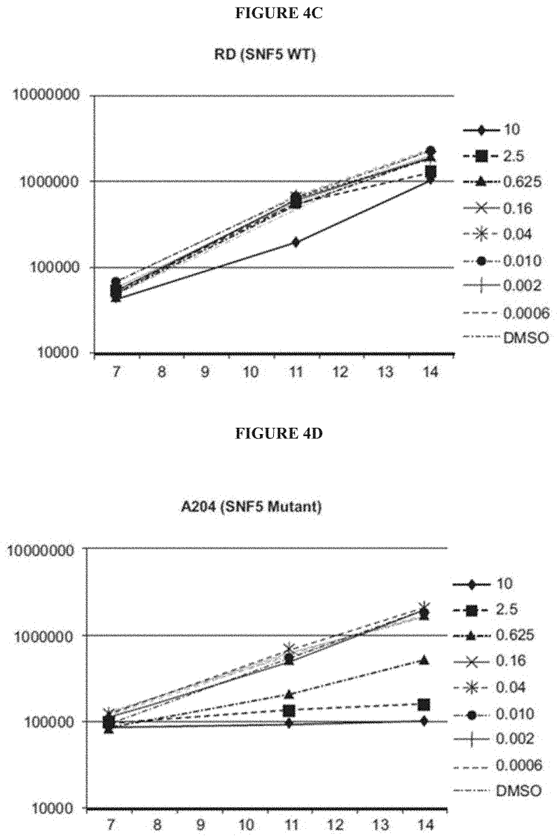

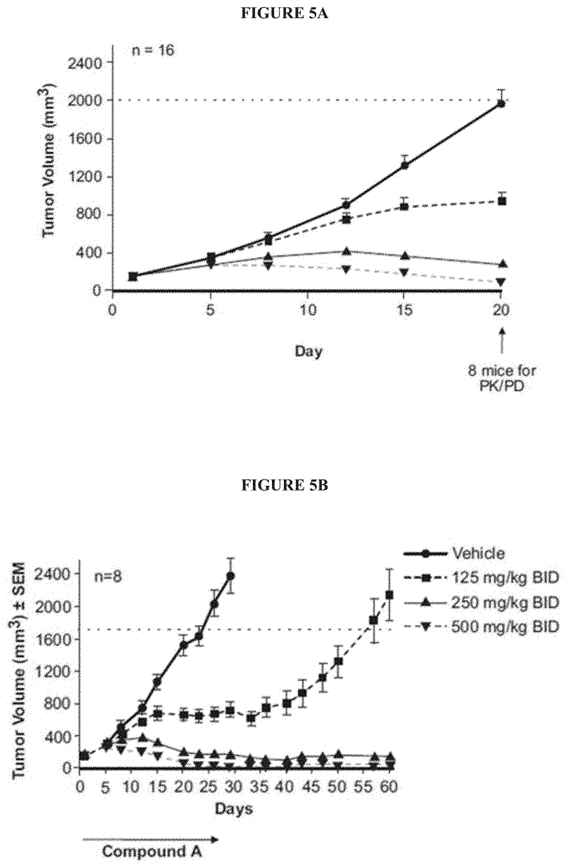

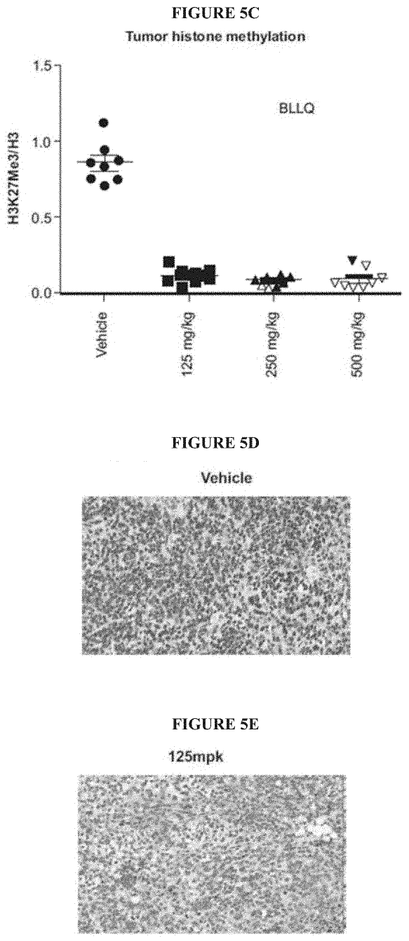

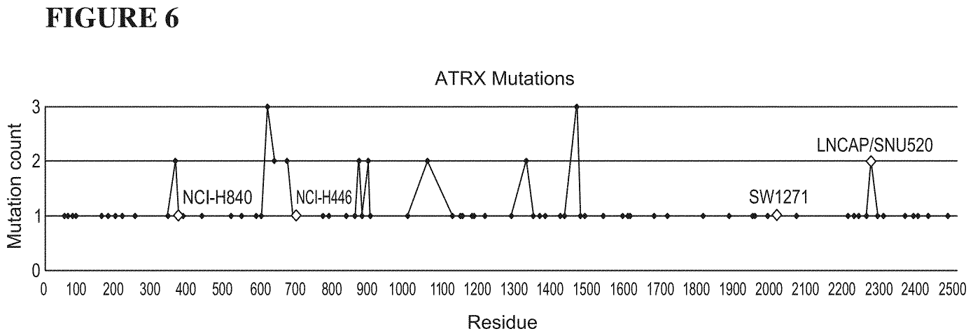

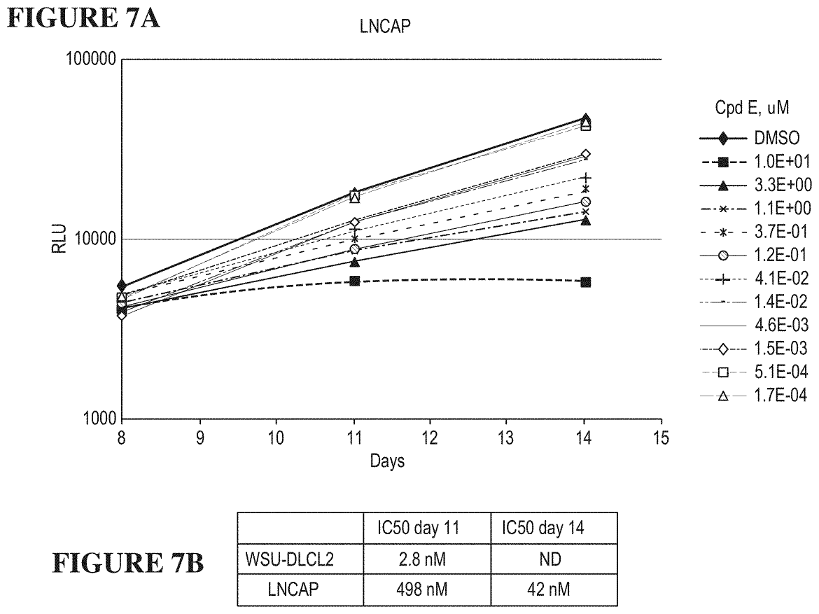

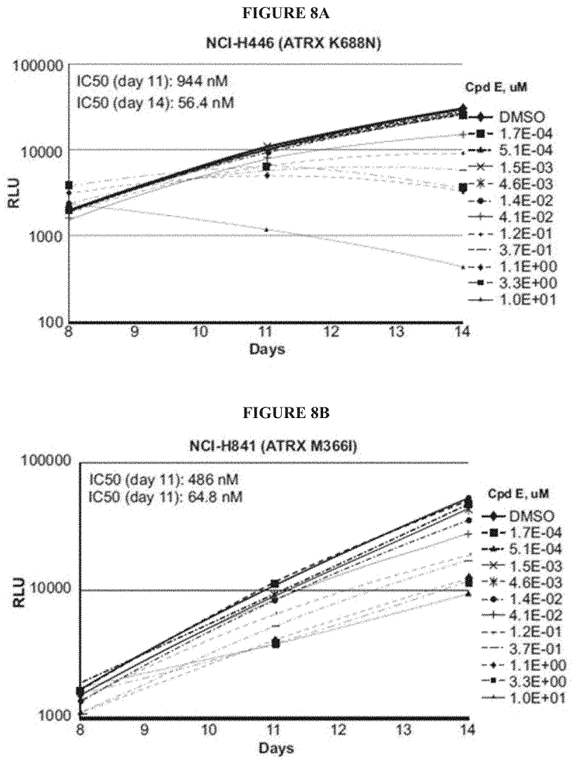

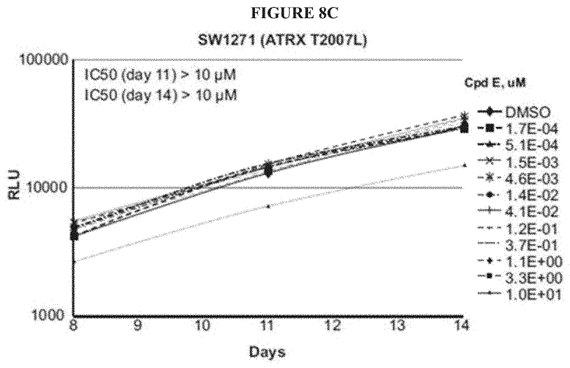

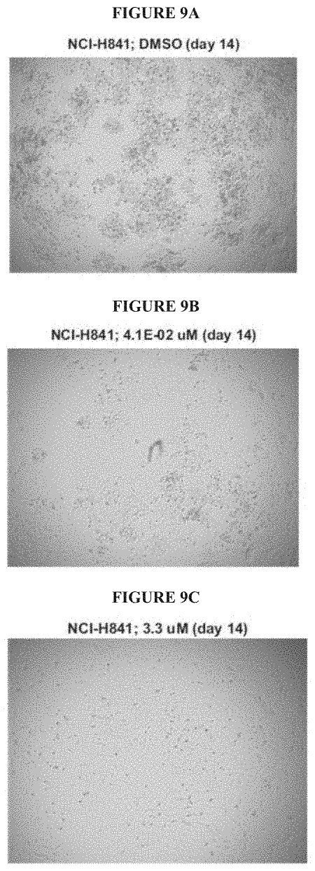

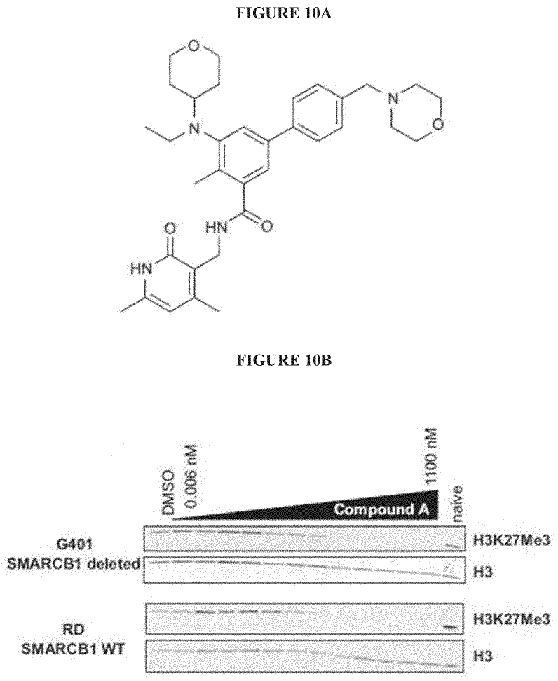

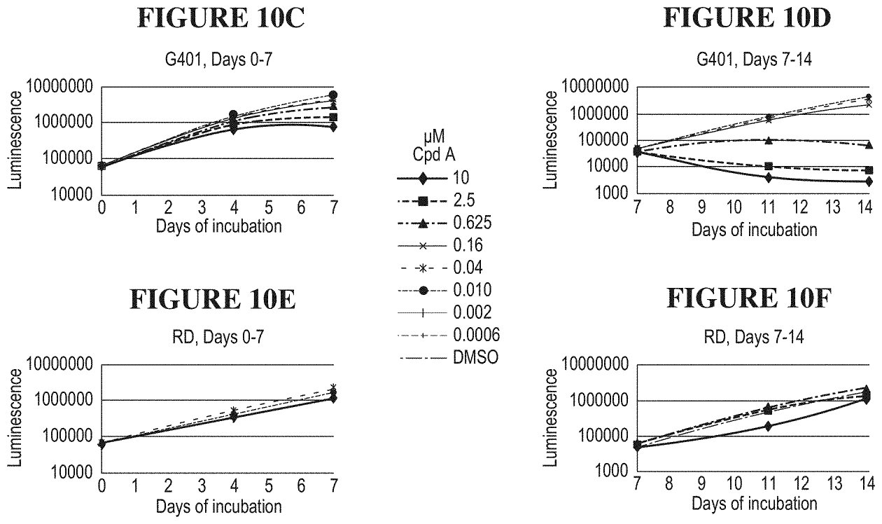

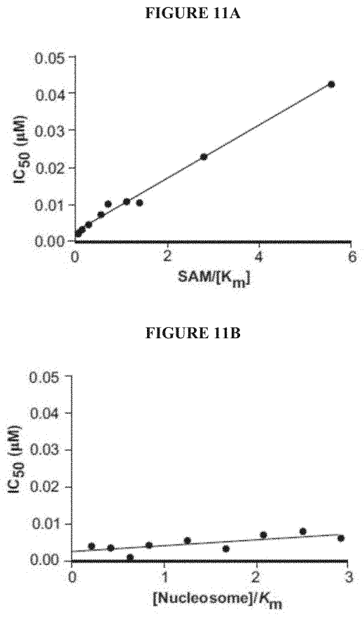

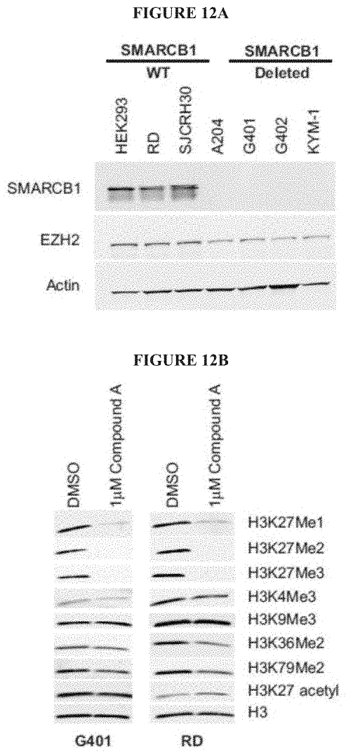

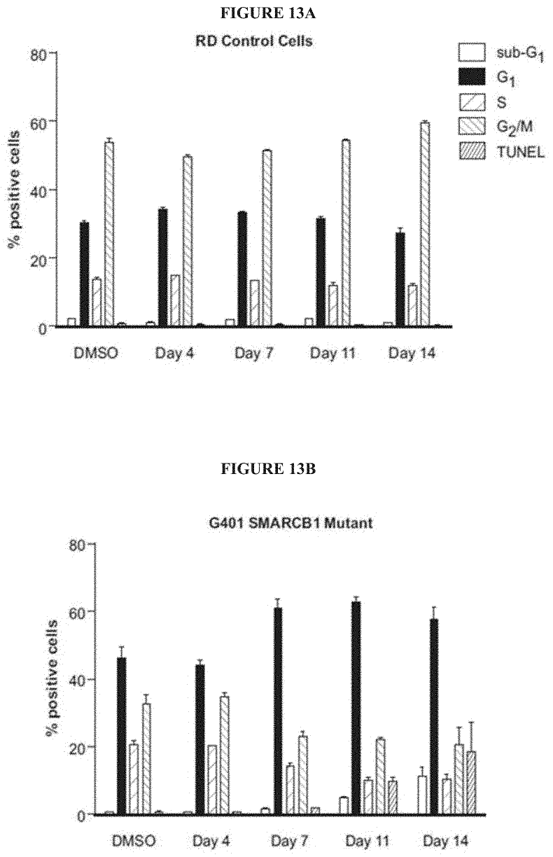

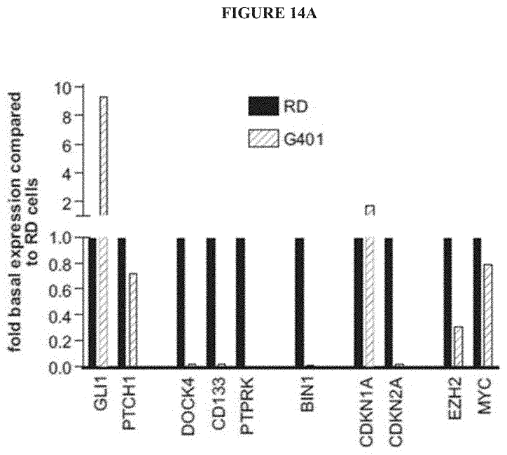

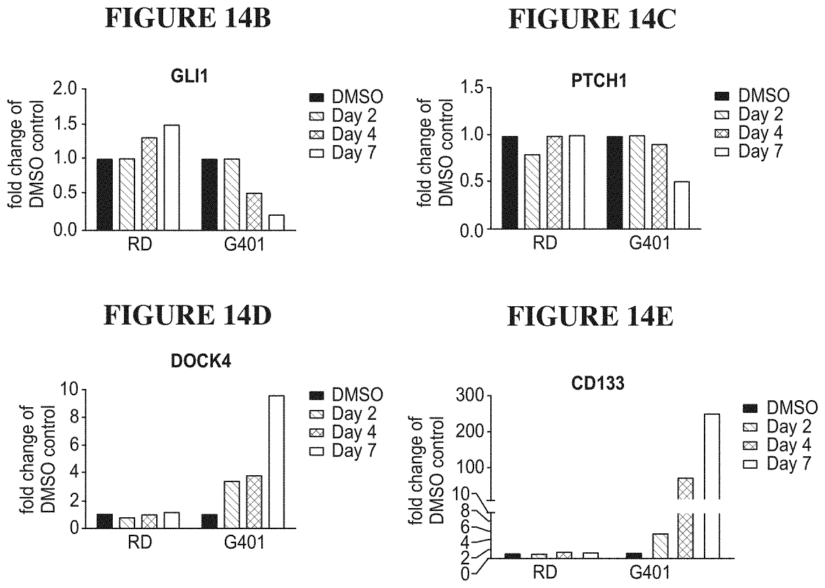

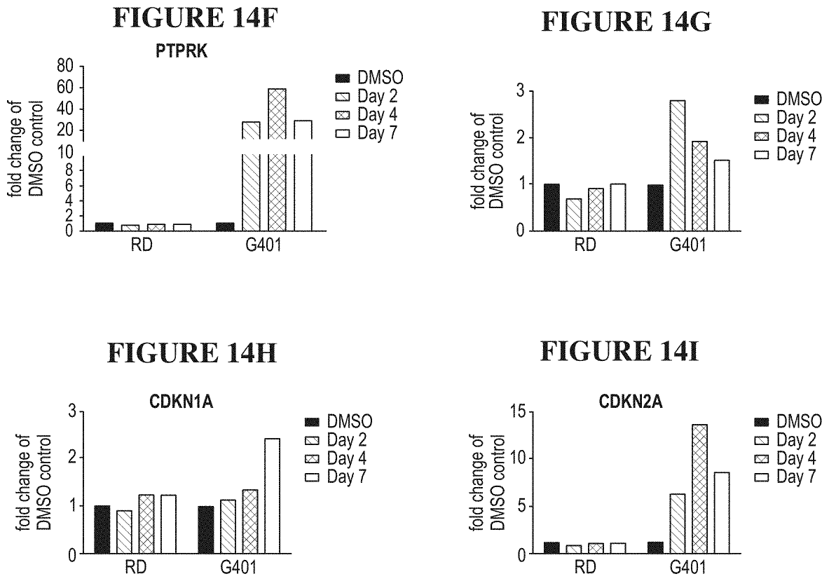

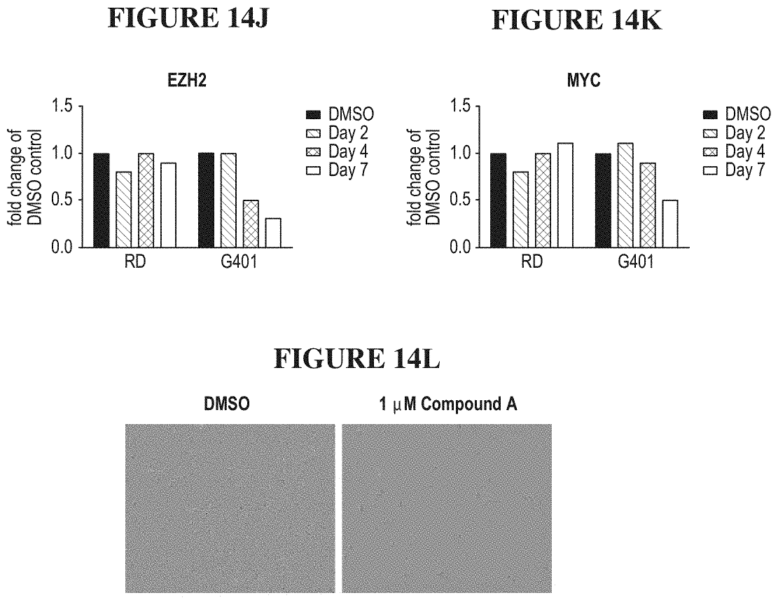

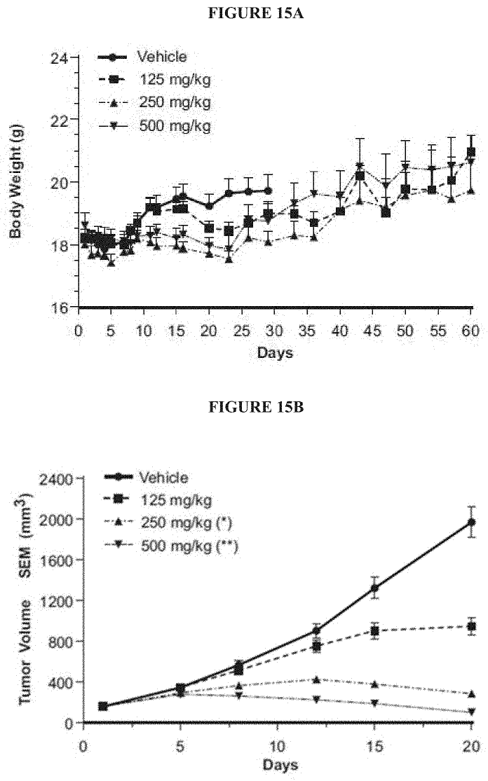

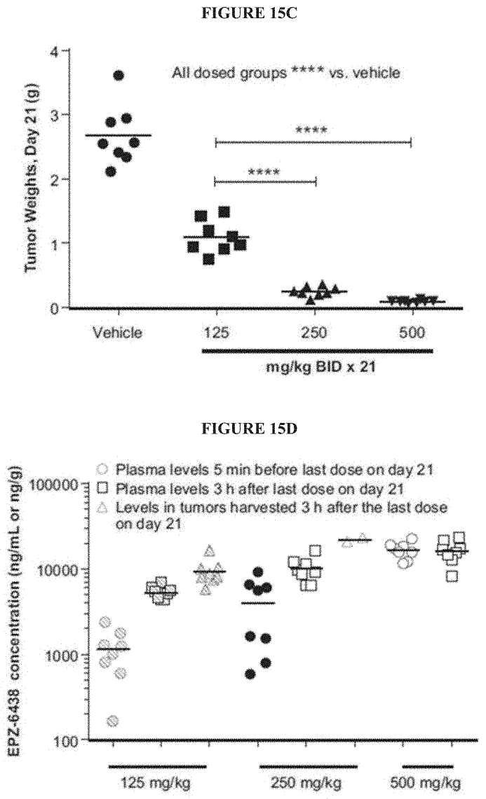

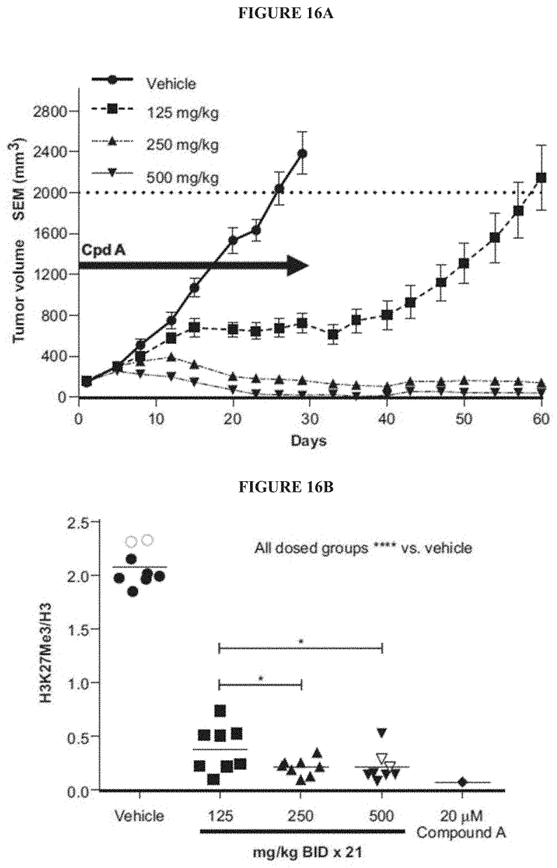

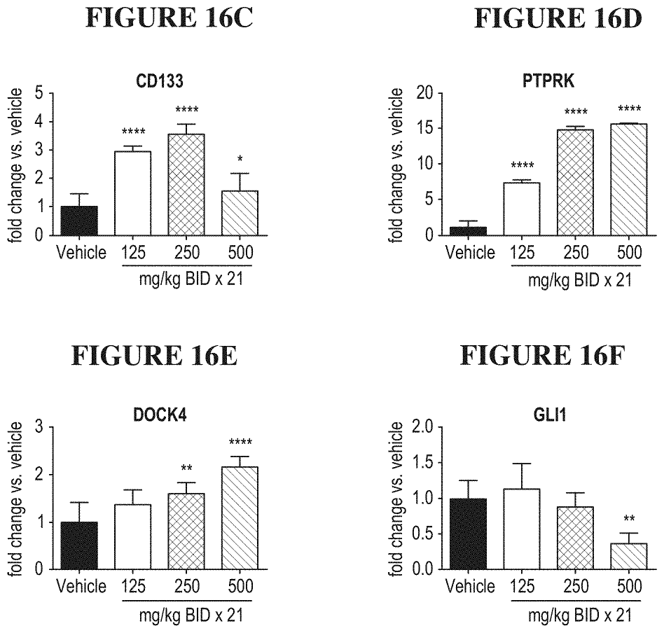

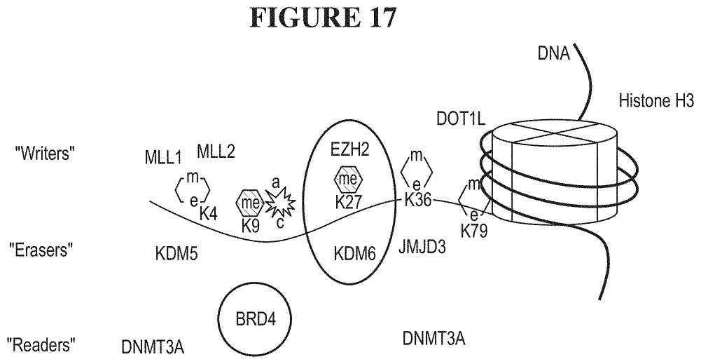

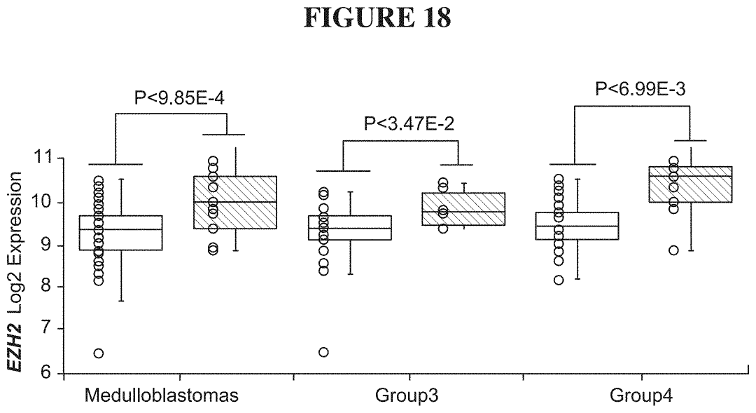

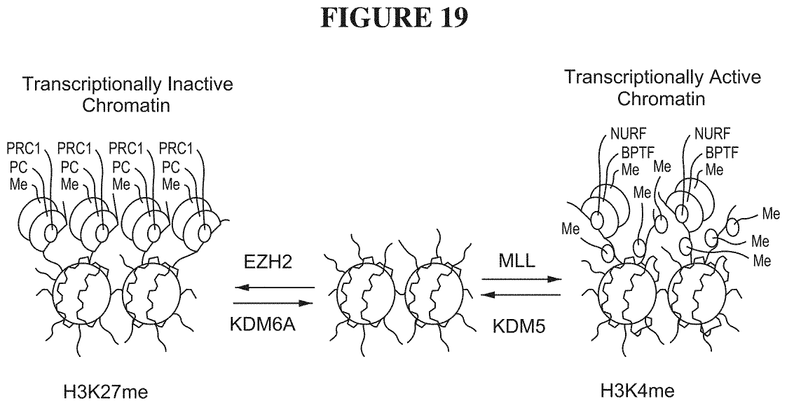

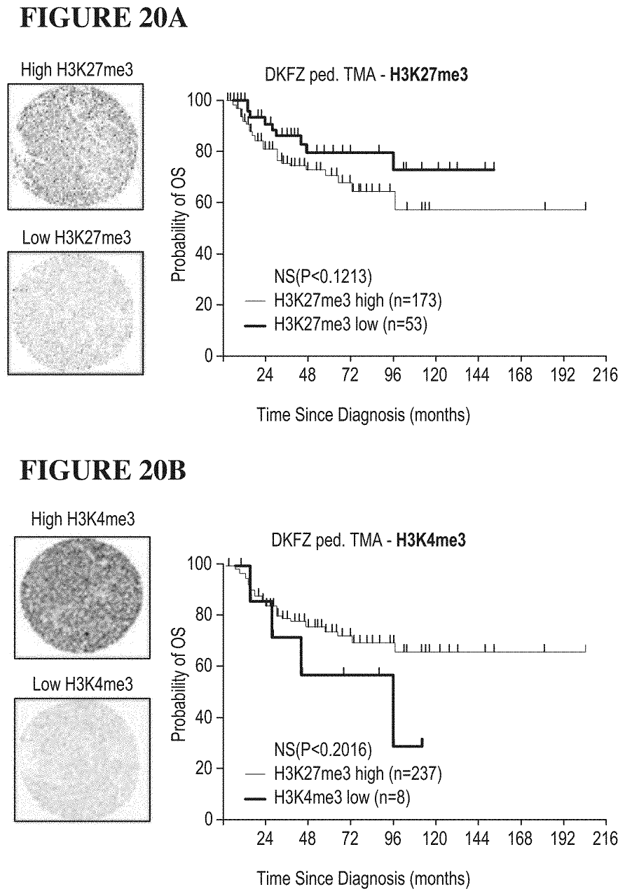

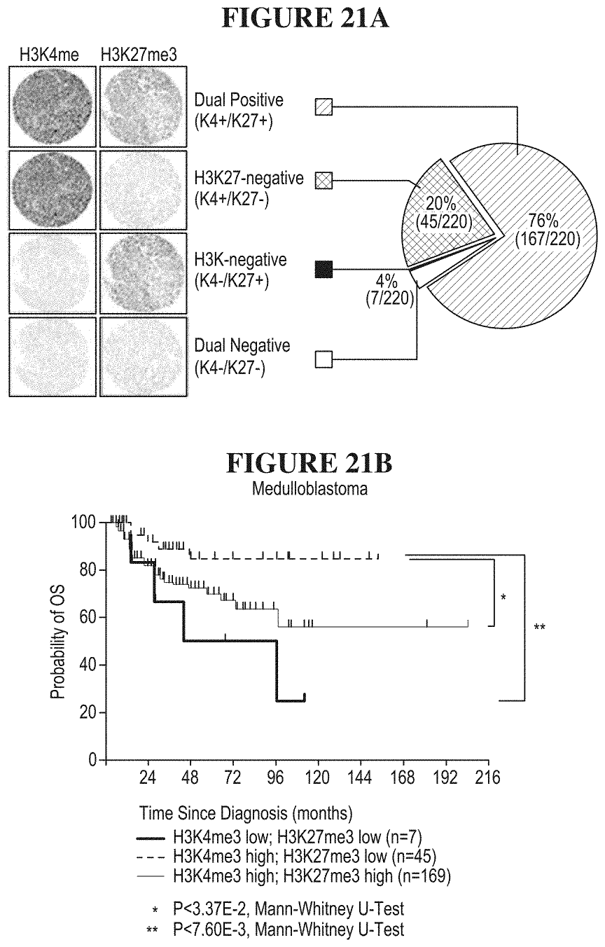

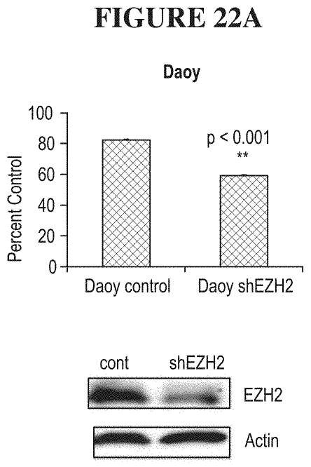

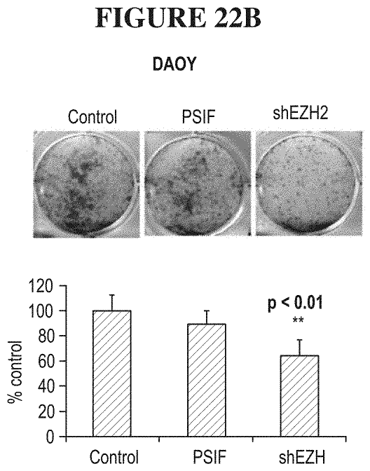

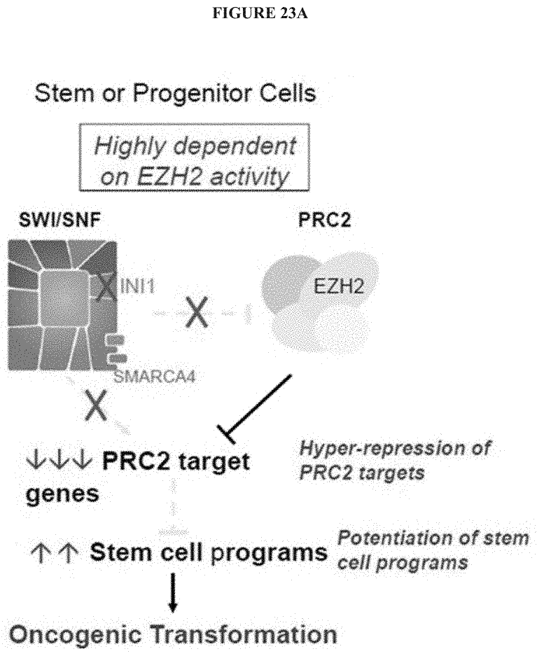

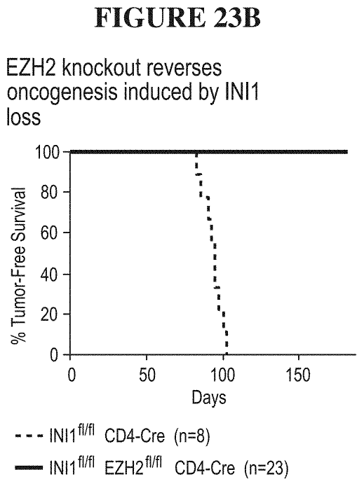

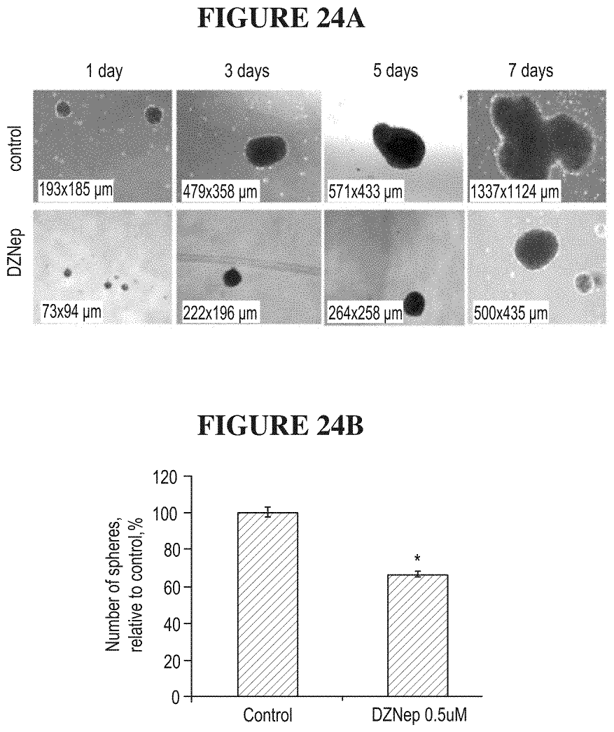

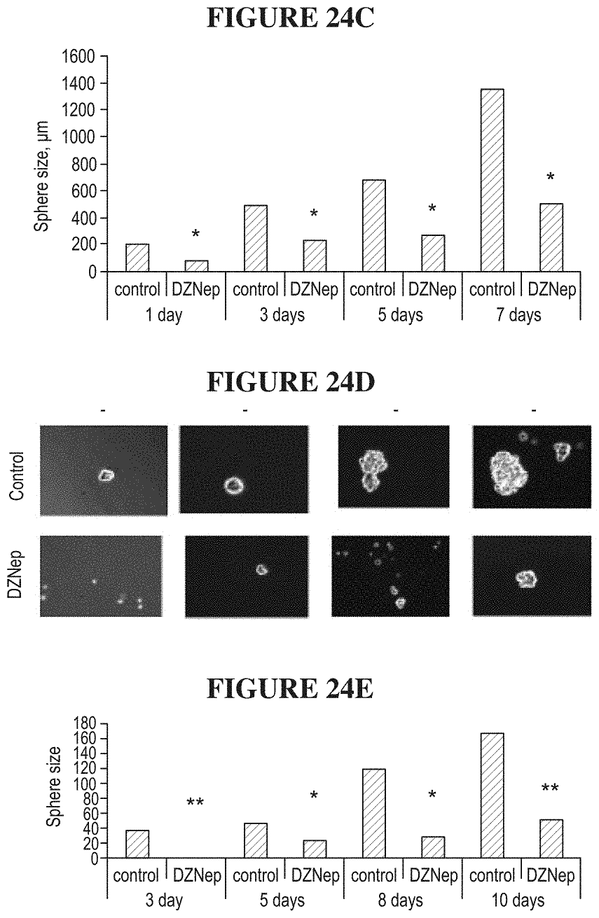

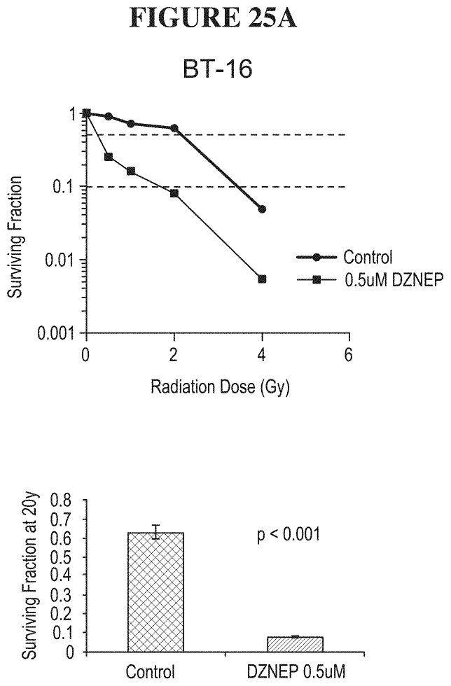

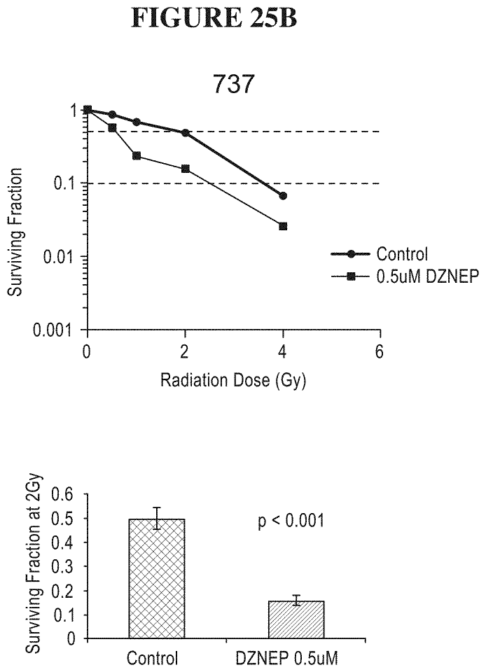

### DETAILED DESCRIPTION

Some aspects of this disclosure provide methods, strategies, and dosing schedules for treating cancer in a subject by administering to the subject a therapeutically-effective amount of an enhancer of a zeste homolog 2 (EZH2) inhibitor. In some embodiments, the cancer is an INI1-deficient tumor. In some embodiments, methods of treating cancer, e.g., an INI1-deficient tumor, of the disclosure may comprise preventing and/or inhibiting proliferation of a malignant cell, e.g., of an INI1-deficient cell.

The disclosure provides a method for treating or alleviating a symptom of a SWI/SNF-associated cancer in a subject by administering to a subject in need thereof a therapeutically effective amount of an EZH2 inhibitor. For example, the SWPSNF-associated cancer is characterized by reduced expression and/or loss of function of the SWI/SNF complex or one or more components of the SWI/SNF complex. In a preferred embodiment, the cancer is an INI1-deficient tumor

The disclosure also provides a method of treating or alleviating a symptom of a SWPSNF-associated cancer in a subject in need thereof by (a) determining the expression level of at least one gene selected from the group consisting of differentiation genes, cell cycle inhibition genes and tumor suppressor genes in a sample obtained from the subject; (b) selecting the subject having a decreased expression level of at least one gene in step a; and (c) administering to the subject selected in step b an effective amount of an EZH2 inhibitor, thereby treating or alleviating a symptom of cancer in the subject. In a preferred embodiment, the cancer is an INI1-deficient tumor.

The disclosure further provides a method of treating or alleviating a symptom of a SWPSNF-associated cancer in a subject in need thereof by (a) determining the expression level of at least one gene selected from the group consisting of hedgehog pathway genes, myc pathway genes and histone methyltransferase genes in a sample obtained from the subject; (b) selecting the subject having an increased expression level of at least one gene in step a; and (c) administering to the subject selected in step b an effective amount of an EZH2 inhibitor, thereby treating or alleviating a symptom of cancer in the subject. In a preferred embodiment, the cancer is an INI1-deficient tumor.

For example, the differentiation gene is CD133, DOCK4, or PTPRK.

For example, the cell cycle inhibition gene is CKDN1A or CDKN2A.

For example, the tumor suppressor gene is BIN1.

For example, the hedgehog pathway gene is GLI1 or PTCH1.

For example, the myc pathway gene is MYC.

For example, the histone methyltransferase gene is EZH2.

The disclosure also provides a method of inducing differentiation, cell cycle inhibition or tumor suppression by contacting a cell with an EZH2 inhibitor. The EZH2 inhibitor may be in an amount sufficient to increase expression of at least one gene selected from the group consisting of CD133, DOCK4, PTPRK, CKDN1A, CDKN2A and BIN1.

The disclosure also provides a method of inhibiting hedgehog signaling by contacting a cell with an EZH2 inhibitor. The EZH2 inhibitor can be in an amount sufficient to reduce expression of GLI1 and/or PTCH1.

The disclosure also provides a method of inducing gene expression by contacting a cell with an EZH2 inhibitor. The EZH2 inhibitor can be in an amount sufficient to induce differentiation, cell cycle inhibition and/or tumor suppression. For example, the gene can be CD133, DOCK4, PTPRK, CKDN1A, CKDN2A or BIN1.

The disclosure also provides a method of inhibiting gene expression by contacting a cell with an EZH2 inhibitor. The EZH2 inhibitor is in an amount sufficient to inhibit hedgehog signaling. For example, the gene can be GLI1 or PTCH1.

For example, the cell may have loss of function of SNF5, ARID1A, ATRX, and/or a component of the SWI/SNF complex.

For example, the loss of function is caused by a deletion of SNF5.

For example, the cell is a cancer cell. Preferably, the cancer is an INI1-deficient cancer cell.

For example, the EZH2 inhibitor comprises

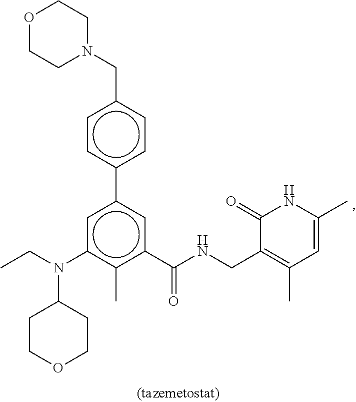

or a pharmaceutically-acceptable salt thereof.

For example, the EZH2 inhibitor comprises

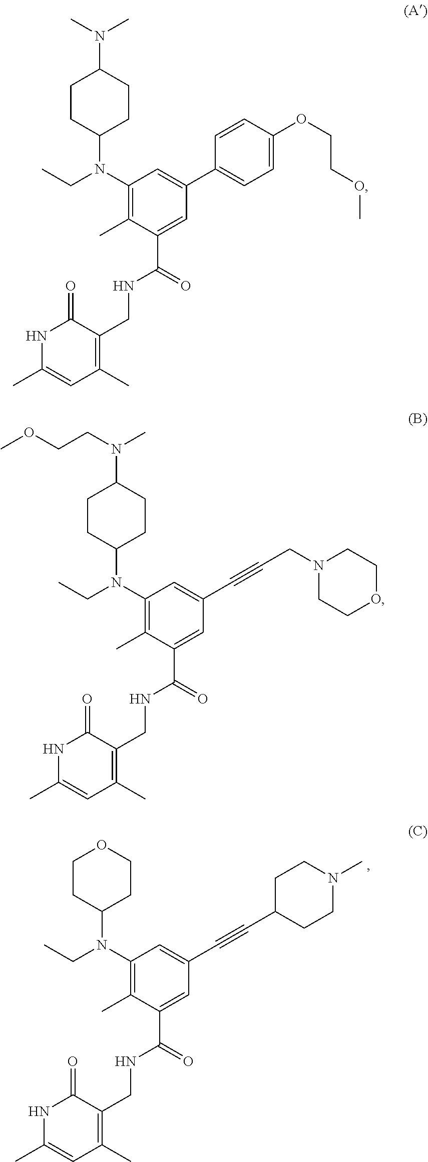

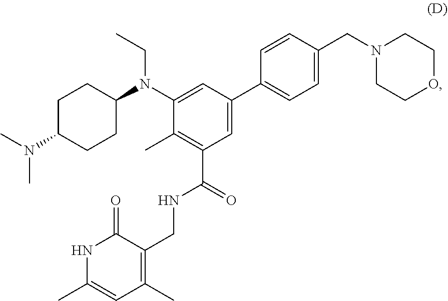 [SMILES: CCN(c1cc(-c2ccc(CN3CCOCC3)cc2)cc(C(=O)NCc2c(C)cc(C)[nH]c2=O)c1C)[C@H]1CC[C@H](N(C)C)CC1]

a stereoisomer, a pharmaceutically acceptable salt and/or a solvate thereof.

For example, the EZH2 inhibitor comprises

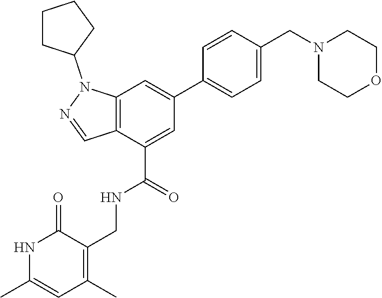 [SMILES: Cc1cc(C)c(CNC(=O)c2cc(-c3ccc(CN4CCOCC4)cc3)cc3c2cnn3C2CCCC2)c(=O)[nH]1]

or a pharmaceutically acceptable salt thereof.

For example, the EZH2 inhibitor comprises

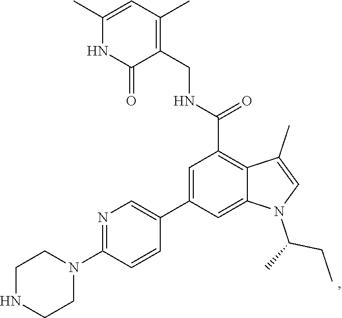 [SMILES: CC[C@H](C)n1cc(C)c2c(C(=O)NCc3c(C)cc(C)[nH]c3=O)cc(-c3ccc(N4CCNCC4)nc3)cc21]

a stereoisomer, a pharmaceutically acceptable salt and/or a solvate thereof.

For example, the EZH2 inhibitor comprises

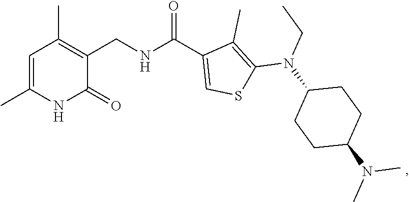

a stereoisomer, a pharmaceutically acceptable salt and/or a solvate thereof.

For example, the EZH2 inhibitor comprises

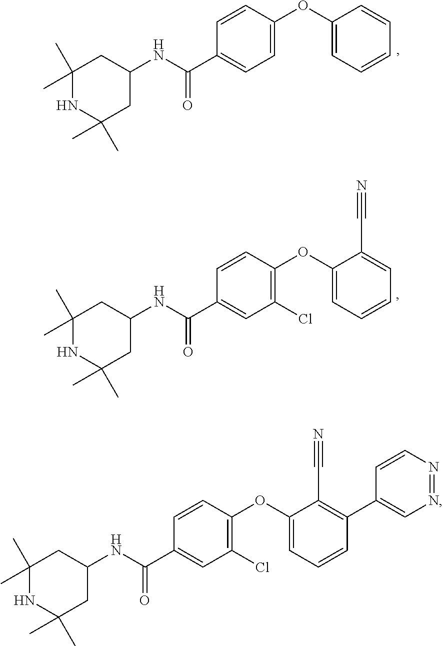 [SMILES: CC1(C)CC(NC(=O)c2ccc(Oc3ccccc3)cc2)CC(C)(C)N1 ; CC1(C)CC(NC(=O)c2ccc(Oc3ccccc3C#N)c(Cl)c2)CC(C)(C)N1 ; CC1(C)CC(NC(=O)c2ccc(Oc3cccc(-c4ccnnc4)c3C#N)c(Cl)c2)CC(C)(C)N1]

a stereoisomer, a pharmaceutically acceptable salt and/or a solvate thereof.

Human nucleic acid and amino acid sequence of components of the SWI/SNF complex have previously been described. See, e.g., GenBank Accession Nos NP_003064.2, NM_003073.3, NP_001007469.1, and NM_001007468.1 for SNF5, GenBank Accession Nos NM_000489.3, NP_000480.2, NM_138270.2, and NP_612114.1 for ATRX, GenBank Accession Nos NP_006006.3, NM_006015.4, NP_624361.1, and NM_139135.2 for ARID1A, each of which is incorporated herein by reference in its entirety.

Spectrum of hSNF5 somatic mutations in human has also been described in Sevenet et al., Human Molecular Genetics, 8: 2359-2368, 1999, which is incorporated herein by reference in its entirety.

A subject in need thereof may have reduced expression, haploinsufficiency, and/or loss of function of SNF5. For example, a subject can comprise a deletion of SNF5 in SNF5 polypeptide or a nucleic acid sequence encoding a SNF5 polypeptide.

| SWI/SNF-related matrix-associated actin-dependent regulator of chromatin subfamily B | |
|---|---|
| member 1 isoform a (SMARCB1, also called SNF5)[Homo sapiens] | |
| (SEQ ID NO: 1) | |
| 1 | mmmmalsktf gqkpvkfqle ddgefymigs evgnylrmfr gslykrypsl wrrlatveer |
| 61 | kkivasshgk ktkpntkdhg yttlatsvtl lkaseveeil dgndekykav sistepptyl |
| 121 | reqkakrnsq wvptlpnssh hldavpcstt inrnrmgrdk krtfplcfdd hdpavihena |
| 181 | sqpevlvpir ldmeidgqkl rdaftwnmne klmtpemfse ilcddldlnp ltfvpaiasa |
| 241 | irqqiesypt dsiledqsdq rviiklnihv gnislvdqfe wdmsekensp ekfalklcse |
| 301 | lglggefvtt iaysirgqls whqktyafse nplptveiai rntgdadqwc plletltdae |
| 361 | mekkirdqdr ntrrmrrlan tapaw |
| Homo sapiens SWI/SNF related, matrix associated, actin dependent regulator of chromatin, | |
| subfamily b, member 1 (SMARCB1, also called SNF5), transcript variant 1, mRNA | |
| (SEQ ID NO: 2) | |
| 1 | aacgccagcg cctgcgcact gagggcggcc tggtcgtcgt ctgcggcggc ggcggcggct |
| 61 | gaggagcccg gctgaggcgc cagtacccgg cccggtccgc atttcgcctt ccggcttcgg |
| 121 | tttccctcgg cccagcacgc cccggccccg ccccagccct cctgatccct cgcagcccgg |
| 181 | ctccggccgc ccgcctctgc cgccgcaatg atgatgatgg cgctgagcaa gaccttcggg |
| 241 | cagaagcccg tgaagttcca gctggaggac gacggcgagt tctacatgat cggctccgag |
| 301 | gtgggaaact acctccgtat gttccgaggt tctctgtaca agagataccc ctcactctgg |
| 361 | aggcgactag ccactgtgga agagaggaag aaaatagttg catcgtcaca tggtaaaaaa |
| 421 | acaaaaccta acactaagga tcacggatac acgactctag ccaccagtgt gaccctgtta |
| 481 | aaagcctcgg aagtggaaga gattctggat ggcaacgatg agaagtacaa ggctgtgtcc |
| 541 | atcagcacag agccccccac ctacctcagg gaacagaagg ccaagaggaa cagccagtgg |
| 601 | gtacccaccc tgcccaacag ctcccaccac ttagatgccg tgccatgctc cacaaccatc |
| 661 | aacaggaacc gcatgggccg agacaagaag agaaccttcc ccctttgctt tgatgaccat |
| 721 | gacccagctg tgatccatga gaacgcatct cagcccgagg tgctggtccc catccggctg |
| 781 | gacatggaga tcgatgggca gaagctgcga gacgccttca cctggaacat gaatgagaag |
| 841 | ttgatgacgc ctgagatgtt ttcagaaatc ctctgtgacg atctggattt gaacccgctg |
| 901 | acgtttgtgc cagccatcgc ctctgccatc agacagcaga tcgagtccta ccccacggac |
| 961 | agcatcctgg aggaccagtc agaccagcgc gtcatcatca agctgaacat ccatgtggga |
| 1021 | aacatttccc tggtggacca gtttgagtgg gacatgtcag agaaggagaa ctcaccagag |
| 1081 | aagtttgccc tgaagctgtg ctcggagctg gggttgggcg gggagtttgt caccaccatc |
| 1141 | gcatacagca tccggggaca gctgagctgg catcagaaga cctacgcctt cagcgagaac |
| 1201 | cctctgccca cagtggagat tgccatccgg aacacgggcg atgcggacca gtggtgccca |
| 1261 | ctgctggaga ctctgacaga cgctgagatg gagaagaaga tccgcgacca ggacaggaac |
| 1321 | acgaggcgga tgaggcgtct tgccaacacg gccccggcct ggtaaccagc ccatcagcac |
| 1381 | acggctccca cggagcatct cagaagattg ggccgcctct cctccatctt ctggcaagga |
| 1441 | cagaggcgag gggacagccc agcgccatcc tgaggatcgg gtgggggtgg agtgggggct |
| 1501 | tccaggtggc ccttcccggc acacattcca tttgttgagc cccagtcctg ccccccaccc |
| 1561 | caccctccct acccctcccc agtctctggg gtcaggaaga aaccttattt taggttgtgt |
| 1621 | tttgtttttg tataggagcc ccaggcaggg ctagtaacag tttttaaata aaaggcaaca |
| 1681 | ggtcatgttc aatttcttca acaaaaaaaa aaaaaaa |
| SWI/SNF-related matrix-associated actin-dependent regulator of chromatin subfamily B | |
| member 1 isoform b [Homo sapiens](SMARCB1, also called SNF5) | |
| (SEQ ID NO: 3) | |
| 1 | mmmmalsktf gqkpvkfqle ddgefymigs evgnylrmfr gslykrypsl wrrlatveer |
| 61 | kkivasshdh gyttlatsvt llkaseveei ldgndekyka vsisteppty lreqkakrns |
| 121 | qwvptlpnss hhldavpcst tinrnrmgrd kkrtfplcfd dhdpavihen asqpevlvpi |
| 181 | rldmeidgqk lrdaftwnmn eklmtpemfs eilcddldln pltfvpaias airqqiesyp |
| 241 | tdsiledqsd qrviiklnih vgnislvdqf ewdmsekens pekfalklcs elglggefvt |
| 301 | tiaysirgql swhqktyafs enplptveia irntgdadqw cplletltda emekkirdqd |
| 361 | rntrrmrrla ntapaw |
| Homo sapiens SWI/SNF related, matrix associated, actin dependent regulator of chromatin, | |
| subfamily b, member 1 (SMARCB1, also called SNF5), transcript variant 2, mRNA | |
| (SEQ ID NO: 4) | |
| 1 | aacgccagcg cctgcgcact gagggcggcc tggtcgtcgt ctgcggcggc ggcggcggct |
| 61 | gaggagcccg gctgaggcgc cagtacccgg cccggtccgc atttcgcctt ccggcttcgg |
| 121 | tttccctcgg cccagcacgc cccggccccg ccccagccct cctgatccct cgcagcccgg |
| 181 | ctccggccgc ccgcctctgc cgccgcaatg atgatgatgg cgctgagcaa gaccttcggg |
| 241 | cagaagcccg tgaagttcca gctggaggac gacggcgagt tctacatgat cggctccgag |
| 301 | gtgggaaact acctccgtat gttccgaggt tctctgtaca agagataccc ctcactctgg |
| 361 | aggcgactag ccactgtgga agagaggaag aaaatagttg catcgtcaca tgatcacgga |
| 421 | tacacgactc tagccaccag tgtgaccctg ttaaaagcct cggaagtgga agagattctg |
| 481 | gatggcaacg atgagaagta caaggctgtg tccatcagca cagagccccc cacctacctc |
| 541 | agggaacaga aggccaagag gaacagccag tgggtaccca ccctgcccaa cagctcccac |
| 601 | cacttagatg ccgtgccatg ctccacaacc atcaacagga accgcatggg ccgagacaag |
| 661 | aagagaacct tccccctttg ctttgatgac catgacccag ctgtgatcca tgagaacgca |
| 721 | tctcagcccg aggtgctggt ccccatccgg ctggacatgg agatcgatgg gcagaagctg |
| 781 | cgagacgcct tcacctggaa catgaatgag aagttgatga cgcctgagat gttttcagaa |
| 841 | atcctctgtg acgatctgga tttgaacccg ctgacgtttg tgccagccat cgcctctgcc |
| 901 | atcagacagc agatcgagtc ctaccccacg gacagcatcc tggaggacca gtcagaccag |
| 961 | cgcgtcatca tcaagctgaa catccatgtg ggaaacattt ccctggtgga ccagtttgag |
| 1021 | tgggacatgt cagagaagga gaactcacca gagaagtttg ccctgaagct gtgctcggag |
| 1081 | ctggggttgg gcggggagtt tgtcaccacc atcgcataca gcatccgggg acagctgagc |
| 1141 | tggcatcaga agacctacgc cttcagcgag aaccctctgc ccacagtgga gattgccatc |
| 1201 | cggaacacgg gcgatgcgga ccagtggtgc ccactgctgg agactctgac agacgctgag |
| 1261 | atggagaaga agatccgcga ccaggacagg aacacgaggc ggatgaggcg tcttgccaac |
| 1321 | acggccccgg cctggtaacc agcccatcag cacacggctc ccacggagca tctcagaaga |
| 1381 | ttgggccgcc tctcctccat cttctggcaa ggacagaggc gaggggacag cccagcgcca |
| 1441 | tcctgaggat cgggtggggg tggagtgggg gcttccaggt ggcccttccc ggcacacatt |
| 1501 | ccatttgttg agccccagtc ctgcccccca ccccaccctc cctacccctc cccagtctct |
| 1561 | ggggtcagga agaaacctta ttttaggttg tgttttgttt ttgtatagga gccccaggca |
| 1621 | gggctagtaa cagtttttaa ataaaaggca acaggtcatg ttcaatttct tcaacaaaaa |
| 1681 | aaaaaaaaaa |

A subject in need thereof may have reduced expression, haploinsufficiency, and/or loss of function of ATRX. For example, a subject can comprise a mutation selected from the group consisting of a substitution of asparagine (N) for the wild type residue lysine (K) at amino acid position 688 of SEQ ID NO: 5 (K688N), and a substitution of isoleucine (I) for the wild type residue methionine (M) at amino acid position 366 of SEQ ID NO: 5 (M366I).

| Homo sapiens alpha thalassemia/mental retardation syndrome X-linked (ATRX) isoform 1 | |
|---|---|
| (SEQ ID NO: 5) | |
| 1 | mtaepmsesk lntlvqklhd flahsseese etsspprlam nqntdkisgs gsnsdmmens |
| 61 | keegtsssek skssgssrsk rkpsivtkyv esddekpldd etvnedasne nsenditmqs |
| 121 | 1pkgtvivqp epvlnedkdd fkgpefrsrs kmktenlkkr gedglhgivs ctacgqqvnh |
| 181 | fqkdsiyrhp slqvlicknc fkyymsddis rdsdgmdeqc rwcaeggnli ccdfchnafc |
| 241 | kkcilrnlgr kelstimden nqwycyichp eplldlvtac nsvfenleql lqqnkkkikv |
| 301 | dseksnkvye htsrfspkkt ssncngeekk lddscsgsvt ysysalivpk emikkakkli |
| 361 | ettanmnssy vkflkqatdn seissatklr qlkafksvla dikkahlale edlnsefram |
| 421 | davnkekntk ehkvidakfe tkarkgekpc alekkdisks eaklsrkqvd sehmhqnvpt |
| 481 | eeqrtnkstg gehkksdrke epqyepants edldmdivsv pssvpedife nletamevqs |
| 541 | svdhqgdgss gteqevesss vklnisskdn rggiksktta kvtkelyvkl tpvslsnspi |
| 601 | kgadcqevpq dkdgykscgl npklekcglg qensdnehlv enevslllee sdlrrsprvk |
| 661 | ttplrrptet npvtsnsdee cnetvkekqk lsvpvrkkdk rnssdsaidn pkpnklpksk |
| 721 | qsetvdqnsd sdemlailke vsrmshssss dtdineihtn hktlydlktq agkddkgkrk |
| 781 | rksstsgsdf dtkkgksaks siiskkkrqt qsessnydse lekeiksmsk igaarttkkr |
| 841 | ipntkdfdss edekhskkgm dnqghknlkt sqegssddae rkqeretfss aegtvdkdtt |
| 901 | imelrdrlpk kqqasastdg vdklsgkeqs ftslevrkva etkekskhlk tktckkvgdg |
| 961 | lsdiaekflk kdqsdetsed dkkqskkgte ekkkpsdfkk kvikmeqqye sssdgteklp |
| 1021 | ereeichfpk gikqikngtt dgekkskkir dktskkkdel sdyaekstgk gdscdssedk |
| 1081 | kskngaygre kkrckllgks srkrqdcsss dtekysmked gcnssdkrlk rielrerrnl |
| 1141 | sskrntkeiq sgssssdaee ssednkkkkq rtsskkkavi vkekkrnslr tstkrkqadi |
| 1201 | tsssssdied ddqnsigegs sdeqkikpvt enlvlsshtg fcqssgdeal sksvpvtvdd |
| 1261 | ddddndpenr iakkmlleei kanlssdedg ssddepeegk krtgkqneen pgdeeaknqv |
| 1321 | nsesdsdsee skkpryrhrl lrhkltvsdg esgeekktkp kehkevkgrn rrkvssedse |
| 1381 | dsdfqesgvs eevsesedeq rprtrsakka eleenqrsyk qkkkrrrikv qedsssenks |
| 1441 | nseeeeeeke eeeeeeeeee eeeedendds kspgkgrkki rkilkddklr tetqnalkee |
| 1501 | eerrkriaer erereklrev ieiedasptk cpittklvld edeetkeplv qvhrnmvikl |
| 1561 | kphqvdgvqf mwdcccesvk ktkkspgsgc ilahcmglgk tlqvvsflht vllcdkldfs |
| 1621 | talvvcplnt alnwmnefek wqeglkddek levselatvk rpqersymlq rwqedggvmi |
| 1681 | igyemyrnla qgrnvksrkl keifnkalvd pgpdfvvcde ghilkneasa vskamnsirs |
| 1741 | rrriiltgtp lqnnlieyhc mvnfikenll gsikefrnrf inpigngqca dstmvdvrvm |
| 1801 | kkrahilyem lagcvqrkdy taltkflppk heyvlavrmt siqcklyqyy ldhltgvgnn |
| 1861 | seggrgkaga klfqdfqmls riwthpwclq ldyiskenkg yfdedsmdef iasdsdetsm |
| 1921 | slssddytkk kkkgkkgkkd ssssgsgsdn dvevikvwns rsrgggegnv detgnnpsys |
| 1981 | lkleeskats ssnpsspapd wykdfvtdad aevlehsgkm vllfeilrma eeigdkvlvf |
| 2041 | sqslisldli edflelasre ktedkdkpli ykgegkwlrn idyyrldgst taqsrkkwae |
| 2101 | efndetnvrg rlfiistkag slginlvaan rviifdaswn psydiqsifr vyrfgqtkpv |
| 2161 | yvyrflaqgt medkiydrqv tkqslsfrvv dqqqverhft mneltelytf epdllddpns |
| 2221 | ekkkkrdtpm lpkdtilael lqihkehivg yhehdslldh keeeelteee rkaawaeyea |
| 2281 | ekkgltmrfn iptgtnlppv sfnsqtpyip fnlgalsams nqqledlinq grekvveatn |
| 2341 | svtavriqpl ediisavwke nmnlseaqvg alalsrqasq eldvkrreai yndvltkqqm |
| 2401 | liscvqrilm nrrlqqqynq qqqqqmtyqg atlghlmmpk ppnlimnpsn yqqidmrgmy |
| 2461 | qpvaggmqpp plqrapppmr sknpgpsqgk sm |
| Homo sapiens alpha thalassemia/mental retardation syndrome X-linked (ATRX), transcript | |
| variant 1, mRNA | |
| (SEQ ID NO: 6) | |
| 1 | aattctcctg cctgagcctc ggcccaacaa aatggcggcg gcagcggtgt cgctttgttt |
| 61 | ccgcggctcc tgcggcggtg gcagtggtag cggcctttga gctgtgggga ggttccagca |
| 121 | gcagctacag tgacgactaa gactccagtg catttctatc gtaaccgggc gcgggggagc |
| 181 | gcagatcggc gcccagcaat cacagaagcc gacaaggcgt tcaagcgaaa acatgaccgc |
| 241 | tgagcccatg agtgaaagca agttgaatac attggtgcag aagcttcatg acttccttgc |
| 301 | acactcatca gaagaatctg aagaaacaag ttctcctcca cgacttgcaa tgaatcaaaa |
| 361 | cacagataaa atcagtggtt ctggaagtaa ctctgatatg atggaaaaca gcaaggaaga |
| 421 | gggaactagc tcttcagaaa aatccaagtc ttcaggatcg tcacgatcaa agaggaaacc |
| 481 | ttcaattgta acaaagtatg tagaatcaga tgatgaaaaa cctttggatg atgaaactgt |
| 541 | aaatgaagat gcgtctaatg aaaattcaga aaatgatatt actatgcaga gcttgccaaa |
| 601 | aggtacagtg attgtacagc cagagccagt gctgaatgaa gacaaagatg attttaaagg |
| 661 | gcctgaattt agaagcagaa gtaaaatgaa aactgaaaat ctcaaaaaac gcggagaaga |
| 721 | tgggcttcat gggattgtga gctgcactgc ttgtggacaa caggtcaatc attttcaaaa |
| 781 | agattccatt tatagacacc cttcattgca agttcttatt tgtaagaatt gctttaagta |
| 841 | ttacatgagt gatgatatta gccgtgactc agatggaatg gatgaacaat gtaggtggtg |
| 901 | tgcggaaggt ggaaacttga tttgttgtga cttttgccat aatgctttct gcaagaaatg |
| 961 | cattctacgc aaccttggtc gaaaggagtt gtccacaata atggatgaaa acaaccaatg |
| 1021 | gtattgctac atttgtcacc cagagccttt gttggacttg gtcactgcat gtaacagcgt |
| 1081 | atttgagaat ttagaacagt tgttgcagca aaataagaag aagataaaag ttgacagtga |
| 1141 | aaagagtaat aaagtatatg aacatacatc cagattttct ccaaagaaga ctagttcaaa |
| 1201 | ttgtaatgga gaagaaaaga aattagatga ttcctgttct ggctctgtaa cctactctta |
| 1261 | ttccgcacta attgtgccca aagagatgat taagaaggca aaaaaactga ttgagaccac |
| 1321 | agccaacatg aactccagtt atgttaaatt tttaaagcag gcaacagata attcagaaat |
| 1381 | cagttctgct acaaaattac gtcagcttaa ggcttttaag tctgtgttgg ctgatattaa |
| 1441 | gaaggctcat cttgcattgg aagaagactt aaattccgag tttcgagcga tggatgctgt |
| 1501 | aaacaaagag aaaaatacca aagagcataa agtcatagat gctaagtttg aaacaaaagc |
| 1561 | acgaaaagga gaaaaacctt gtgctttgga aaagaaggat atttcaaagt cagaagctaa |
| 1621 | actttcaaga aaacaggtag atagtgagca catgcatcag aatgttccaa cagaggaaca |
| 1681 | aagaacaaat aaaagtaccg gtggtgaaca taagaaatct gatagaaaag aagaacctca |
| 1741 | atatgaacct gccaacactt ctgaagattt agacatggat attgtgtctg ttccttcctc |
| 1801 | agttccagaa gacatttttg agaatcttga gactgctatg gaagttcaga gttcagttga |
| 1861 | tcatcaaggg gatggcagca gtggaactga acaagaagtg gagagttcat ctgtaaaatt |
| 1921 | aaatatttct tcaaaagaca acagaggagg tattaaatca aaaactacag ctaaagtaac |
| 1981 | aaaagaatta tatgttaaac tcactcctgt ttccctttct aattccccaa ttaaaggtgc |
| 2041 | tgattgtcag gaagttccac aagataaaga tggctataaa agttgtggtc tgaaccccaa |
| 2101 | gttagagaaa tgtggacttg gacaggaaaa cagtgataat gagcatttgg ttgaaaatga |
| 2161 | agtttcatta cttttagagg aatctgatct tcgaagatcc ccacgtgtaa agactacacc |
| 2221 | cttgaggcga ccgacagaaa ctaaccctgt aacatctaat tcagatgaag aatgtaatga |
| 2281 | aacagttaag gagaaacaaa aactatcagt tccagtgaga aaaaaggata agcgtaattc |
| 2341 | ttctgacagt gctatagata atcctaagcc taataaattg ccaaaatcta agcaatcaga |
| 2401 | gactgtggat caaaattcag attctgatga aatgctagca atcctcaaag aggtgagcag |
| 2461 | gatgagtcac agttcttctt cagatactga tattaatgaa attcatacaa accataagac |
| 2521 | tttgtatgat ttaaagactc aggcggggaa agatgataaa ggaaaaagga aacgaaaaag |
| 2581 | ttctacatct ggctcagatt ttgatactaa aaagggcaaa tcagctaaga gctctataat |
| 2641 | ttctaaaaag aaacgacaaa cccagtctga gtcttctaat tatgactcag aattagaaaa |
| 2701 | agagataaag agcatgagta aaattggtgc tgccagaacc accaaaaaaa gaattccaaa |
| 2761 | tacaaaagat tttgactctt ctgaagatga gaaacacagc aaaaaaggaa tggataatca |
| 2821 | agggcacaaa aatttgaaga cctcacaaga aggatcatct gatgatgctg aaagaaaaca |
| 2881 | agagagagag actttctctt cagcagaagg cacagttgat aaagacacga ccatcatgga |
| 2941 | attaagagat cgacttccta agaagcagca agcaagtgct tccactgatg gtgtcgataa |
| 3001 | gctttctggg aaagagcaga gttttacttc tttggaagtt agaaaagttg ctgaaactaa |
| 3061 | agaaaagagc aagcatctca aaaccaaaac atgtaaaaaa gtacaggatg gcttatctga |
| 3121 | tattgcagag aaattcctaa agaaagacca gagcgatgaa acttctgaag atgataaaaa |
| 3181 | gcagagcaaa aagggaactg aagaaaaaaa gaaaccttca gactttaaga aaaaagtaat |
| 3241 | taaaatggaa caacagtatg aatcttcatc tgatggcact gaaaagttac ctgagcgaga |
| 3301 | agaaatttgt cattttccta agggcataaa acaaattaag aatggaacaa ctgatggaga |
| 3361 | aaagaaaagt aaaaaaataa gagataaaac ttctaaaaag aaggatgaat tatctgatta |
| 3421 | tgctgagaag tcaacaggga aaggagatag ttgtgactct tcagaggata aaaagagtaa |
| 3481 | gaatggagca tatggtagag agaagaaaag gtgcaagttg cttggaaaga gttcaaggaa |
| 3541 | gagacaagat tgttcatcat ctgatactga gaaatattcc atgaaagaag atggttgtaa |
| 3601 | ctcttctgat aagagactga aaagaataga attgagggaa agaagaaatt taagttcaaa |
| 3661 | gagaaatact aaggaaatac aaagtggctc atcatcatct gatgctgagg aaagttctga |
| 3721 | agataataaa aagaagaagc aaagaacttc atctaaaaag aaggcagtca ttgtcaagga |
| 3781 | gaaaaagaga aactccctaa gaacaagcac taaaaggaag caagctgaca ttacatcctc |
| 3841 | atcttcttct gatatagaag atgatgatca gaattctata ggtgagggaa gcagcgatga |
| 3901 | acagaaaatt aagcctgtga ctgaaaattt agtgctgtct tcacatactg gattttgcca |
| 3961 | atcttcagga gatgaagcct tatctaaatc agtgcctgtc acagtggatg atgatgatga |
| 4021 | cgacaatgat cctgagaata gaattgccaa gaagatgctt ttagaagaaa ttaaagccaa |
| 4081 | tctttcctct gatgaggatg gatcttcaga tgatgagcca gaagaaggga aaaaaagaac |
| 4141 | tggaaaacaa aatgaagaaa acccaggaga tgaggaagca aaaaatcaag tcaattctga |
| 4201 | atcagattca gattctgaag aatctaagaa gccaagatac agacataggc ttttgcggca |
| 4261 | caaattgact gtgagtgacg gagaatctgg agaagaaaaa aagacaaagc ctaaagagca |
| 4321 | taaagaagtc aaaggcagaa acagaagaaa ggtgagcagt gaagattcag aagattctga |
| 4381 | ttttcaggaa tcaggagtta gtgaagaagt tagtgaatcc gaagatgaac agcggcccag |
| 4441 | aacaaggtct gcaaagaaag cagagttgga agaaaatcag cggagctata aacagaaaaa |
| 4501 | gaaaaggcga cgtattaagg ttcaagaaga ttcatccagt gaaaacaaga gtaattctga |
| 4561 | ggaagaagag gaggaaaaag aagaggagga ggaagaggag gaggaggagg aagaggagga |
| 4621 | ggaagatgaa aatgatgatt ccaagtctcc tggaaaaggc agaaagaaaa ttcggaagat |
| 4681 | tcttaaagat gataaactga gaacagaaac acaaaatgct cttaaggaag aggaagagag |
| 4741 | acgaaaacgt attgctgaga gggagcgtga gcgagaaaaa ttgagagagg tgatagaaat |
| 4801 | tgaagatgct tcacccacca agtgtccaat aacaaccaag ttggttttag atgaagatga |
| 4861 | agaaaccaaa gaacctttag tgcaggttca tagaaatatg gttatcaaat tgaaacccca |
| 4921 | tcaagtagat ggtgttcagt ttatgtggga ttgctgctgt gagtctgtga aaaaaacaaa |
| 4981 | gaaatctcca ggttcaggat gcattcttgc ccactgtatg ggccttggta agactttaca |
| 5041 | ggtggtaagt tttcttcata cagttctttt gtgtgacaaa ctggatttca gcacggcgtt |
| 5101 | agtggtttgt cctcttaata ctgctttgaa ttggatgaat gaatttgaga agtggcaaga |
| 5161 | gggattaaaa gatgatgaga agcttgaggt ttctgaatta gcaactgtga aacgtcctca |
| 5221 | ggagagaagc tacatgctgc agaggtggca agaagatggt ggtgttatga tcataggcta |
| 5281 | tgagatgtat agaaatcttg ctcaaggaag gaatgtgaag agtcggaaac ttaaagaaat |
| 5341 | atttaacaaa gctttggttg atccaggccc tgattttgtt gtttgtgatg aaggccatat |
| 5401 | tctaaaaaat gaagcatctg ctgtttctaa agctatgaat tctatacgat caaggaggag |
| 5461 | gattatttta acaggaacac cacttcaaaa taacctaatt gagtatcatt gtatggttaa |
| 5521 | ttttatcaag gaaaatttac ttggatccat taaggagttc aggaatagat ttataaatcc |
| 5581 | aattcaaaat ggtcagtgtg cagattctac catggtagat gtcagagtga tgaaaaaacg |
| 5641 | tgctcacatt ctctatgaga tgttagctgg atgtgttcag aggaaagatt atacagcatt |
| 5701 | aacaaaattc ttgcctccaa aacacgaata tgtgttagct gtgagaatga cttctattca |
| 5761 | gtgcaagctc tatcagtact acttagatca cttaacaggt gtgggcaata atagtgaagg |
| 5821 | tggaagagga aaggcaggtg caaagctttt ccaagatttt cagatgttaa gtagaatatg |
| 5881 | gactcatcct tggtgtttgc agctagacta cattagcaaa gaaaataagg gttattttga |
| 5941 | tgaagacagt atggatgaat ttatagcctc agattctgat gaaacctcca tgagtttaag |
| 6001 | ctccgatgat tatacaaaaa agaagaaaaa agggaaaaag gggaaaaaag atagtagctc |
| 6061 | aagtggaagt ggcagtgaca atgatgttga agtgattaag gtctggaatt caagatctcg |
| 6121 | gggaggtggt gaaggaaatg tggatgaaac aggaaacaat ccttctgttt ctttaaaact |
| 6181 | ggaagaaagt aaagctactt cttcttctaa tccaagcagc ccagctccag actggtacaa |
| 6241 | agattttgtt acagatgctg atgctgaggt tttagagcat tctgggaaaa tggtacttct |
| 6301 | ctttgaaatt cttcgaatgg cagaggaaat tggggataaa gtccttgttt tcagccagtc |
| 6361 | cctcatatct ctggacttga ttgaagattt tcttgaatta gctagtaggg agaagacaga |
| 6421 | agataaagat aaacccctta tttataaagg tgaggggaag tggcttcgaa acattgacta |
| 6481 | ttaccgttta gatggttcca ctactgcaca gtcaaggaag aagtgggctg aagaatttaa |
| 6541 | tgatgaaact aatgtgagag gacgattatt tatcatttct actaaagcag gatctctagg |
| 6601 | aattaatctg gtagctgcta atcgagtaat tatattcgac gcttcttgga atccatctta |
| 6661 | tgacatccag agtatattca gagtttatcg ctttggacaa actaagcctg tttatgtata |
| 6721 | taggttctta gctcagggaa ccatggaaga taagatttat gatcggcaag taactaagca |
| 6781 | gtcactgtct tttcgagttg ttgatcagca gcaggtggag cgtcatttta ctatgaatga |
| 6841 | gcttactgaa ctttatactt ttgagccaga cttattagat gaccctaatt cagaaaagaa |
| 6901 | gaagaagagg gatactccca tgctgccaaa ggataccata cttgcagagc tccttcagat |
| 6961 | acataaagaa cacattgtag gataccatga acatgattct cttttggacc acaaagaaga |
| 7021 | agaagagttg actgaagaag aaagaaaagc agcttgggct gagtatgaag cagagaagaa |
| 7081 | gggactgacc atgcgtttca acataccaac tgggaccaat ttaccccctg tcagtttcaa |
| 7141 | ctctcaaact ccttatattc ctttcaattt gggagccctg tcagcaatga gtaatcaaca |
| 7201 | gctggaggac ctcattaatc aaggaagaga aaaagttgta gaagcaacaa acagtgtgac |
| 7261 | agcagtgagg attcaacctc ttgaggatat aatttcagct gtatggaagg agaacatgaa |
| 7321 | tctctcagag gcccaagtac aggcgttagc attaagtaga caagccagcc aggagcttga |
| 7381 | tgttaaacga agagaagcaa tctacaatga tgtattgaca aaacaacaga tgttaatcag |
| 7441 | ctgtgttcag cgaatactta tgaacagaag gctccagcag cagtacaatc agcagcaaca |
| 7501 | gcaacaaatg acttatcaac aagcaacact gggtcacctc atgatgccaa agcccccaaa |
| 7561 | tttgatcatg aatccttcta actaccagca gattgatatg agaggaatgt atcagccagt |
| 7621 | ggctggtggt atgcagccac caccattaca gcgtgcacca cccccaatga gaagcaaaaa |
| 7681 | tccaggacct tcccaaggga aatcaatgtg attttgcact aaaagcttaa tggattgtta |
| 7741 | aaatcataga aagatctttt atttttttag gaatcaatga cttaacagaa ctcaactgta |
| 7801 | taaatagttt ggtcccctta aatgccaatc ttccatatta gttttacttt tttttttttt |
| 7861 | aaatagggca taccatttct tcctgacatt tgtcagtgat gttgcctaga atcttcttac |
| 7921 | acacgctgag tacagaagat atttcaaatt gttttcagtg aaaacaagtc cttccataat |
| 7981 | agtaacaact ccacagattt cctctctaaa tttttatgcc tgcttttagc aaccataaaa |
| 8041 | ttgtcataaa attaataaat ttaggaaaga ataaagattt atatattcat tctttacata |
| 8101 | taaaaacaca cagctgagtt cttagagttg attcctcaag ttatgaaata cttttgtact |
| 8161 | taatccattt cttgattaaa gtgattgaaa tggttttaat gttcttttga ctgaagtctg |
| 8221 | aaactgggct cctgctttat tgtctctgtg actgaaagtt agaaactgag ggttatcttt |
| 8281 | gacacagaat tgtgtgcaat attcttaaat actactgctc taaaagttgg agaagtcttg |
| 8341 | cagttatctt agcattgtat aaacagcctt aagtatagcc taagaagaga attccttttt |
| 8401 | cttctttagt ccttctgcca ttttttattt tcagttatat gtgctgaaat aattactggt |
| 8461 | aaaatttcag ggttgtggat tatcttccac acatgaattt tctctctcct ggcacgaata |
| 8521 | taaagcacat ctcttaactg catggtgcca gtgctaatgc ttcatcctgt tgctggcagt |
| 8581 | gggatgtgga cttagaaaat caagttctag cattttagta ggttaacact gaagttgtgg |
| 8641 | ttgttaggtt cacaccctgt tttataaaca acatcaaaat ggcagaacca ttgctgactt |
| 8701 | taggttcaca tgaggaatgt acttttaaca attcccagta ctatcagtat tgtgaaataa |
| 8761 | ttcctctgaa agataagaat cactggcttc tatgcgcttc ttttctctca tcatcatgtt |
| 8821 | cttttacccc agtttcctta cattttttta aattgtttca gagtttgttt tttttttagt |
| 8881 | ttagattgtg aggcaattat taaatcaaaa ttaattcatc caatacccct ttactagaag |
| 8941 | ttttactaga aaatgtatta cattttattt tttcttaatc cagttctgca aaaatgacct |
| 9001 | ataaatttat tcatgtacaa ttttggttac ttgaattgtt aaagaaaaca ttgtttttga |
| 9061 | ctatgggagt caactcaaca tggcagaacc atttttgaga tgatgataca acaggtagtg |
| 9121 | aaacagctta agaattccaa aaaaaaaaaa aaaaaaaaaa aaaagaaaac tgggtttggg |
| 9181 | ctttgcttta ggtatcactg gattagaatg agtttaacat tagctaaaac tgctttgagt |
| 9241 | tgtttggatg attaagagat tgccattttt atcttggaag aactagtggt aaaacatcca |
| 9301 | agagcactag gattgtgata cagaatttgt gaggtttggt ggatccacgc ccctctcccc |
| 9361 | cactttccca tgatgaaata tcactaataa atcctgtata tttagatatt atgctagcca |
| 9421 | tgtaatcaga tttatttaat tgggtggggc aggtgtgtat ttactttaga aaaaatgaaa |
| 9481 | aagacaagat ttatgagaaa tatttgaagg cagtacactc tggccaactg ttaccagttg |
| 9541 | gtatttctac aagttcagaa tattttaaac ctgatttact agacctggga attttcaaca |
| 9601 | tggtctaatt atttactcaa agacatagat gtgaaaattt taggcaacct tctaaatctt |
| 9661 | tttcaccatg gatgaaacta taacttaaag aataatactt agaagggtta attggaaatc |
| 9721 | agagtttgaa ataaaacttg gaccactttg tatacactct tctcacttga cattttagct |
| 9781 | atataatatg tactttgagt ataacatcaa gctttaacaa atatttaaag acaaaaaaat |
| 9841 | cacgtcagta aaatactaaa aggctcattt ttatatttgt tttagatgtt ttaaatagtt |
| 9901 | gcaatggatt aaaaatgatg atttaaaatg ttgcttgtaa tacagttttg cctgctaaat |
| 9961 | tctccacatt ttgtaacctg ttttatttct ttgggtgtaa agcgtttttg cttagtattg |
| 10021 | tgatattgta tatgttttgt cccagttgta tagtaatgtt tcagtccatc atccagcttt |
| 10081 | ggctgctgaa atcatacagc tgtgaagact tgcctttgtt tctgttagac tgcttttcag |
| 10141 | ttctgtattg agtatcttaa gtactgtaga aaagatgtca cttcttcctt taaggctgtt |
| 10201 | ttgtaatata tataaggact ggaattgtgt ttttaaagaa aagcattcaa gtatgacaat |
| 10261 | atactatctg tgttttcacc attcaaagtg ctgtttagta gttgaaactt aaactattta |
| 10321 | atgtcattta ataaagtgac caaaatgtgt tgtgctcttt attgtatttt cacagctttg |
| 10381 | aaaatctgtg cacatactgt ttcatagaaa atgtatagct tttgttgtcc tatataatgg |
| 10441 | tggttctttt gcacatttag ttatttaata ttgagaggtc acgaagtttg gttattgaat |
| 10501 | ctgttatata ctaaattctg taaagggaga tctctcatct caaaaagaat ttacatacca |
| 10561 | ggaagtccat gtgtgtttgt gttagttttg gatgtctttg tgtaatccag ccccatttcc |
| 10621 | tgtttcccaa cagctgtaac actcatttta agtcaagcag ggctaccaac ccacacttga |
| 10681 | tagaaaagct gcttaccatt cagaagcttc cttattacct ggcctccaaa tgagctgaat |
| 10741 | attttgtagc cttcccttag ctatgttcat tttccctcca ttatcataaa atcagatcga |
| 10801 | tatttatgtg ccccaaacaa aactttaaga gcagttacat tctgtcccag tagcccttgt |
| 10861 | ttcctttgag agtagcatgt tgtgaggcta tagagactta ttctaccagt aaaacaggtc |
| 10921 | aatcctttta catgtttatt atactaaaaa ttatgttcag ggtatttact actttatttc |
| 10981 | accagactca gtctcaagtg acttggctat ctccaaatca gatctaccct tagagaataa |
| 11041 | acatttttct accgttattt tttttcaagt ctataatctg agccagtccc aaaggagtga |
| 11101 | tcaagtttca gaaatgcttt catcttcaca acattttata tatactatta tatggggtga |
| 11161 | ataaagtttt aaatccgaaa tataaaaaaa aaaaaaaaaa aa |
| Homo sapiens alpha thalassemia/mental retardation syndrome X-linked (ATRX) isoforrn 2 | |
| (SEQ ID NO: 7) | |
| 1 | mtaepmsesk lntlvqklhd flahsseese etsspprlam nqntdkisgs gsnsdmmens |
| 61 | keegtsssek skssgssrsk rkpsivtkyv esddekpldd etvnedasne nsenditmqs |
| 121 | lpkedglhgi vsctacgqqv nhfqkdsiyr hpslqvlick ncfkyymsdd isrdsdgmde |
| 181 | qcrwcaeggn liccdfchna fckkcilrnl grkelstimd ennqwycyic hpeplldlvt |
| 241 | acnsvfenle qllqqnkkki kvdseksnkv yehtsrfspk ktssncngee kklddscsgs |
| 301 | vtysysaliv pkemikkakk liettanmns syvkflkqat dnseissatk lrqlkafksv |
| 361 | ladikkahla leedlnsefr amdavnkekn tkehkvidak fetkarkgek pcalekkdis |
| 421 | kseaklsrkg vdsehmhqnv pteeqrtnks tggehkksdr keepqyepan tsedldmdiv |
| 481 | svpssvpedi fenletamev qssvdhqgdg ssgteqeves ssvklnissk dnrggikskt |
| 541 | takvtkelyv kltpvslsns pikgadcqev pqdkdgyksc glnpklekcg lgqensdneh |
| 601 | lvenevslll eesdlrrspr vkttplrrpt etnpvtsnsd eecnetvkek qklsvpvrkk |
| 661 | dkrnssdsai dnpkpnklpk skqsetvdqn sdsdemlail kevsrmshss ssdtdineih |
| 721 | tnhktlydlk tqagkddkgk rkrksstsgs dfdtkkgksa kssiiskkkr qtqsessnyd |
| 781 | selekeiksm skigaarttk kripntkdfd ssedekhskk gmdnqghknl ktsqegssdd |
| 841 | aerkqeretf ssaegtvdkd ttimelrdrl pkkqqasast dgvdklsgke qsftslevrk |
| 901 | vaetkekskh lktktckkvq dglsdiaekf lkkdqsdets eddkkqskkg teekkkpsdf |
| 961 | kkkvikmeqq yesssdgtek lpereeichf pkgikqikng ttdgekkskk irdktskkkd |
| 1021 | elsdyaekst gkgdscdsse dkkskngayg rekkrckllg kssrkrqdcs ssdtekysmk |
| 1081 | edgcnssdkr lkrielrerr nlsskrntke iqsgssssda eessednkkk kqrtsskkka |
| 1141 | vivkekkrns lrtstkrkqa ditsssssdi edddqnsige gssdeqkikp vtenlvlssh |
| 1201 | tgfcqssgde alsksvpvtv ddddddndpe nriakkmlle eikanlssde dgssddepee |
| 1261 | gkkrtgkqne enpgdeeakn qvnsesdsds eeskkpryrh rllrhkltvs dgesgeekkt |
| 1321 | kpkehkevkg rnrrkvssed sedsdfqesg vseevsesed eqrprtrsak kaeleenqrs |
| 1381 | ykqkkkrrri kvqedsssen ksnseeeeee keeeeeeeee eeeeeedend dskspgkgrk |
| 1441 | kirkilkddk lrtetqnalk eeeerrkria ererereklr evieiedasp tkcpittklv |
| 1501 | ldedeetkep lvqvhrnmvi klkphqvdgv qfmwdccces vkktkkspgs gcilahcmgl |
| 1561 | gktlqvvsfl htvllcdkld fstalvvcpl ntalnwmnef ekwqeglkdd eklevselat |
| 1621 | vkrpqersym lqrwqedggv miigyemyrn laqgrnvksr klkeifnkal vdpgpdfvvc |
| 1681 | deghilknea savskamnsi rsrrriiltg tplqnnliey hcmvnfiken llgsikefrn |
| 1741 | rfinpiqngq cadstmvdvr vmkkrahily emlagcvqrk dytaltkflp pkheyvlavr |
| 1801 | mtsiqcklyq yyldhltgvg nnseggrgka gaklfqdfqm lsriwthpwc lqldyisken |
| 1861 | kgyfdedsmd efiasdsdet smslssddyt kkkkkgkkgk kdssssgsgs dndvevikvw |
| 1921 | nsrsrgggeg nvdetgnnps vslkleeska tsssnpsspa pdwykdfvtd adaevlehsg |
| 1981 | kmvllfeilr maeeigdkvl vfsqslisld liedflelas rektedkdkp liykgegkwl |
| 2041 | rnidyyrldg sttaqsrkkw aeefndetnv rgrlfiistk agslginlva anrviifdas |
| 2101 | wnpsydiqsi frvyrfgqtk pvyvyrflaq gtmedkiydr qvtkqslsfr vvdqqqverh |
| 2161 | ftmneltely tfepdllddp nsekkkkrdt pmlpkdtila ellqihkehi vgyhehdsll |
| 2221 | dhkeeeelte eerkaawaey eaekkgltmr fniptgtnlp pvsfnsqtpy ipfnlgalsa |
| 2281 | msnqqledli nqgrekvvea tnsvtavriq plediisavw kenmnlseaq vqalalsrqa |
| 2341 | sqeldvkrre aiyndvltkq qmliscvqri lmnrrlqqqy nqqqqqqmty qqatlghlmm |
| 2401 | pkppnlimnp snyqqidmrg myqpvaggmq ppplqrappp mrsknpgpsq gksm |
| Homo sapiens alpha thalassemia/mental retardation syndrome X-linked (ATRX), transcript | |
| variant 2, mRNA | |
| (SEQ ID NO: 8) | |
| 1 | aattctcctg cctgagcctc ggcccaacaa aatggcggcg gcagcggtgt cgctttgttt |
| 61 | ccgcggctcc tgcggcggtg gcagtggtag cggcctttga gctgtgggga ggttccagca |
| 121 | gcagctacag tgacgactaa gactccagtg catttctatc gtaaccgggc gcgggggagc |
| 181 | gcagatcggc gcccagcaat cacagaagcc gacaaggcgt tcaagcgaaa acatgaccgc |
| 241 | tgagcccatg agtgaaagca agttgaatac attggtgcag aagcttcatg acttccttgc |
| 301 | acactcatca gaagaatctg aagaaacaag ttctcctcca cgacttgcaa tgaatcaaaa |
| 361 | cacagataaa atcagtggtt ctggaagtaa ctctgatatg atggaaaaca gcaaggaaga |
| 421 | gggaactagc tcttcagaaa aatccaagtc ttcaggatcg tcacgatcaa agaggaaacc |
| 481 | ttcaattgta acaaagtatg tagaatcaga tgatgaaaaa cctttggatg atgaaactgt |
| 541 | aaatgaagat gcgtctaatg aaaattcaga aaatgatatt actatgcaga gcttgccaaa |
| 601 | agaagatggg cttcatggga ttgtgagctg cactgcttgt ggacaacagg tcaatcattt |
| 661 | tcaaaaagat tccatttata gacacccttc attgcaagtt cttatttgta agaattgctt |
| 721 | taagtattac atgagtgatg atattagccg tgactcagat ggaatggatg aacaatgtag |
| 781 | gtggtgtgcg gaaggtggaa acttgatttg ttgtgacttt tgccataatg ctttctgcaa |
| 841 | gaaatgcatt ctacgcaacc ttggtcgaaa ggagttgtcc acaataatgg atgaaaacaa |
| 901 | ccaatggtat tgctacattt gtcacccaga gcctttgttg gacttggtca ctgcatgtaa |
| 961 | cagcgtattt gagaatttag aacagttgtt gcagcaaaat aagaagaaga taaaagttga |
| 1021 | cagtgaaaag agtaataaag tatatgaaca tacatccaga ttttctccaa agaagactag |
| 1081 | ttcaaattgt aatggagaag aaaagaaatt agatgattcc tgttctggct ctgtaaccta |
| 1141 | ctcttattcc gcactaattg tgcccaaaga gatgattaag aaggcaaaaa aactgattga |
| 1201 | gaccacagcc aacatgaact ccagttatgt taaattttta aagcaggcaa cagataattc |
| 1261 | agaaatcagt tctgctacaa aattacgtca gcttaaggct tttaagtctg tgttggctga |
| 1321 | tattaagaag gctcatcttg cattggaaga agacttaaat tccgagtttc gagcgatgga |
| 1381 | tgctgtaaac aaagagaaaa ataccaaaga gcataaagtc atagatgcta agtttgaaac |
| 1441 | aaaagcacga aaaggagaaa aaccttgtgc tttggaaaag aaggatattt caaagtcaga |
| 1501 | agctaaactt tcaagaaaac aggtagatag tgagcacatg catcagaatg ttccaacaga |
| 1561 | ggaacaaaga acaaataaaa gtaccggtgg tgaacataag aaatctgata gaaaagaaga |
| 1621 | acctcaatat gaacctgcca acacttctga agatttagac atggatattg tgtctgttcc |
| 1681 | ttcctcagtt ccagaagaca tttttgagaa tcttgagact gctatggaag ttcagagttc |
| 1741 | agttgatcat caaggggatg gcagcagtgg aactgaacaa gaagtggaga gttcatctgt |
| 1801 | aaaattaaat atttcttcaa aagacaacag aggaggtatt aaatcaaaaa ctacagctaa |
| 1861 | agtaacaaaa gaattatatg ttaaactcac tcctgtttcc ctttctaatt ccccaattaa |
| 1921 | aggtgctgat tgtcaggaag ttccacaaga taaagatggc tataaaagtt gtggtctgaa |
| 1981 | ccccaagtta gagaaatgtg gacttggaca ggaaaacagt gataatgagc atttggttga |
| 2041 | aaatgaagtt tcattacttt tagaggaatc tgatcttcga agatccccac gtgtaaagac |
| 2101 | tacacccttg aggcgaccga cagaaactaa ccctgtaaca tctaattcag atgaagaatg |
| 2161 | taatgaaaca gttaaggaga aacaaaaact atcagttcca gtgagaaaaa aggataagcg |
| 2221 | taattcttct gacagtgcta tagataatcc taagcctaat aaattgccaa aatctaagca |
| 2281 | atcagagact gtggatcaaa attcagattc tgatgaaatg ctagcaatcc tcaaagaggt |
| 2341 | gagcaggatg agtcacagtt cttcttcaga tactgatatt aatgaaattc atacaaacca |
| 2401 | taagactttg tatgatttaa agactcaggc ggggaaagat gataaaggaa aaaggaaacg |
| 2461 | aaaaagttct acatctggct cagattttga tactaaaaag ggcaaatcag ctaagagctc |
| 2521 | tataatttct aaaaagaaac gacaaaccca gtctgagtct tctaattatg actcagaatt |
| 2581 | agaaaaagag ataaagagca tgagtaaaat tggtgctgcc agaaccacca aaaaaagaat |
| 2641 | tccaaataca aaagattttg actcttctga agatgagaaa cacagcaaaa aaggaatgga |
| 2701 | taatcaaggg cacaaaaatt tgaagacctc acaagaagga tcatctgatg atgctgaaag |
| 2761 | aaaacaagag agagagactt tctcttcagc agaaggcaca gttgataaag acacgaccat |
| 2821 | catggaatta agagatcgac ttcctaagaa gcagcaagca agtgcttcca ctgatggtgt |
| 2881 | cgataagctt tctgggaaag agcagagttt tacttctttg gaagttagaa aagttgctga |
| 2941 | aactaaagaa aagagcaagc atctcaaaac caaaacatgt aaaaaagtac aggatggctt |
| 3001 | atctgatatt gcagagaaat tcctaaagaa agaccagagc gatgaaactt ctgaagatga |
| 3061 | taaaaagcag agcaaaaagg gaactgaaga aaaaaagaaa ccttcagact ttaagaaaaa |
| 3121 | agtaattaaa atggaacaac agtatgaatc ttcatctgat ggcactgaaa agttacctga |
| 3181 | gcgagaagaa atttgtcatt ttcctaaggg cataaaacaa attaagaatg gaacaactga |
| 3241 | tggagaaaag aaaagtaaaa aaataagaga taaaacttct aaaaagaagg atgaattatc |
| 3301 | tgattatgct gagaagtcaa cagggaaagg agatagttgt gactcttcag aggataaaaa |
| 3361 | gagtaagaat ggagcatatg gtagagagaa gaaaaggtgc aagttgcttg gaaagagttc |
| 3421 | aaggaagaga caagattgtt catcatctga tactgagaaa tattccatga aagaagatgg |
| 3481 | ttgtaactct tctgataaga gactgaaaag aatagaattg agggaaagaa gaaatttaag |
| 3541 | ttcaaagaga aatactaagg aaatacaaag tggctcatca tcatctgatg ctgaggaaag |
| 3601 | ttctgaagat aataaaaaga agaagcaaag aacttcatct aaaaagaagg cagtcattgt |
| 3661 | caaggagaaa aagagaaact ccctaagaac aagcactaaa aggaagcaag ctgacattac |
| 3721 | atcctcatct tcttctgata tagaagatga tgatcagaat tctataggtg agggaagcag |
| 3781 | cgatgaacag aaaattaagc ctgtgactga aaatttagtg ctgtcttcac atactggatt |
| 3841 | ttgccaatct tcaggagatg aagccttatc taaatcagtg cctgtcacag tggatgatga |
| 3901 | tgatgacgac aatgatcctg agaatagaat tgccaagaag atgcttttag aagaaattaa |
| 3961 | agccaatctt tcctctgatg aggatggatc ttcagatgat gagccagaag aagggaaaaa |
| 4021 | aagaactgga aaacaaaatg aagaaaaccc aggagatgag gaagcaaaaa atcaagtcaa |
| 4081 | ttctgaatca gattcagatt ctgaagaatc taagaagcca agatacagac ataggctttt |
| 4141 | gcggcacaaa ttgactgtga gtgacggaga atctggagaa gaaaaaaaga caaagcctaa |
| 4201 | agagcataaa gaagtcaaag gcagaaacag aagaaaggtg agcagtgaag attcagaaga |
| 4261 | ttctgatttt caggaatcag gagttagtga agaagttagt gaatccgaag atgaacagcg |
| 4321 | gcccagaaca aggtctgcaa agaaagcaga gttggaagaa aatcagcgga gctataaaca |
| 4381 | gaaaaagaaa aggcgacgta ttaaggttca agaagattca tccagtgaaa acaagagtaa |
| 4441 | ttctgaggaa gaagaggagg aaaaagaaga ggaggaggaa gaggaggagg aggaggaaga |
| 4501 | ggaggaggaa gatgaaaatg atgattccaa gtctcctgga aaaggcagaa agaaaattcg |
| 4561 | gaagattctt aaagatgata aactgagaac agaaacacaa aatgctctta aggaagagga |
| 4621 | agagagacga aaacgtattg ctgagaggga gcgtgagcga gaaaaattga gagaggtgat |
| 4681 | agaaattgaa gatgcttcac ccaccaagtg tccaataaca accaagttgg ttttagatga |
| 4741 | agatgaagaa accaaagaac ctttagtgca ggttcataga aatatggtta tcaaattgaa |
| 4801 | accccatcaa gtagatggtg ttcagtttat gtgggattgc tgctgtgagt ctgtgaaaaa |
| 4861 | aacaaagaaa tctccaggtt caggatgcat tcttgcccac tgtatgggcc ttggtaagac |
| 4921 | tttacaggtg gtaagttttc ttcatacagt tcttttgtgt gacaaactgg atttcagcac |
| 4981 | ggcgttagtg gtttgtcctc ttaatactgc tttgaattgg atgaatgaat ttgagaagtg |
| 5041 | gcaagaggga ttaaaagatg atgagaagct tgaggtttct gaattagcaa ctgtgaaacg |
| 5101 | tcctcaggag agaagctaca tgctgcagag gtggcaagaa gatggtggtg ttatgatcat |
| 5161 | aggctatgag atgtatagaa atcttgctca aggaaggaat gtgaagagtc ggaaacttaa |
| 5221 | agaaatattt aacaaagctt tggttgatcc aggccctgat tttgttgttt gtgatgaagg |
| 5281 | ccatattcta aaaaatgaag catctgctgt ttctaaagct atgaattcta tacgatcaag |
| 5341 | gaggaggatt attttaacag gaacaccact tcaaaataac ctaattgagt atcattgtat |
| 5401 | ggttaatttt atcaaggaaa atttacttgg atccattaag gagttcagga atagatttat |
| 5461 | aaatccaatt caaaatggtc agtgtgcaga ttctaccatg gtagatgtca gagtgatgaa |
| 5521 | aaaacgtgct cacattctct atgagatgtt agctggatgt gttcagagga aagattatac |
| 5581 | agcattaaca aaattcttgc ctccaaaaca cgaatatgtg ttagctgtga gaatgacttc |
| 5641 | tattcagtgc aagctctatc agtactactt agatcactta acaggtgtgg gcaataatag |
| 5701 | tgaaggtgga agaggaaagg caggtgcaaa gcttttccaa gattttcaga tgttaagtag |
| 5761 | aatatggact catccttggt gtttgcagct agactacatt agcaaagaaa ataagggtta |
| 5821 | ttttgatgaa gacagtatgg atgaatttat agcctcagat tctgatgaaa cctccatgag |
| 5881 | tttaagctcc gatgattata caaaaaagaa gaaaaaaggg aaaaagggga aaaaagatag |
| 5941 | tagctcaagt ggaagtggca gtgacaatga tgttgaagtg attaaggtct ggaattcaag |
| 6001 | atctcgggga ggtggtgaag gaaatgtgga tgaaacagga aacaatcctt ctgtttcttt |
| 6061 | aaaactggaa gaaagtaaag ctacttcttc ttctaatcca agcagcccag ctccagactg |
| 6121 | gtacaaagat tttgttacag atgctgatgc tgaggtttta gagcattctg ggaaaatggt |
| 6181 | acttctcttt gaaattcttc gaatggcaga ggaaattggg gataaagtcc ttgttttcag |
| 6241 | ccagtccctc atatctctgg acttgattga agattttctt gaattagcta gtagggagaa |
| 6301 | gacagaagat aaagataaac cccttattta taaaggtgag gggaagtggc ttcgaaacat |
| 6361 | tgactattac cgtttagatg gttccactac tgcacagtca aggaagaagt gggctgaaga |
| 6421 | atttaatgat gaaactaatg tgagaggacg attatttatc atttctacta aagcaggatc |
| 6481 | tctaggaatt aatctggtag ctgctaatcg agtaattata ttcgacgctt cttggaatcc |
| 6541 | atcttatgac atccagagta tattcagagt ttatcgcttt ggacaaacta agcctgttta |
| 6601 | tgtatatagg ttcttagctc agggaaccat ggaagataag atttatgatc ggcaagtaac |
| 6661 | taagcagtca ctgtcttttc gagttgttga tcagcagcag gtggagcgtc attttactat |
| 6721 | gaatgagctt actgaacttt atacttttga gccagactta ttagatgacc ctaattcaga |
| 6781 | aaagaagaag aagagggata ctcccatgct gccaaaggat accatacttg cagagctcct |
| 6841 | tcagatacat aaagaacaca ttgtaggata ccatgaacat gattctcttt tggaccacaa |
| 6901 | agaagaagaa gagttgactg aagaagaaag aaaagcagct tgggctgagt atgaagcaga |
| 6961 | gaagaaggga ctgaccatgc gtttcaacat accaactggg accaatttac cccctgtcag |
| 7021 | tttcaactct caaactcctt atattccttt caatttggga gccctgtcag caatgagtaa |
| 7081 | tcaacagctg gaggacctca ttaatcaagg aagagaaaaa gttgtagaag caacaaacag |
| 7141 | tgtgacagca gtgaggattc aacctcttga ggatataatt tcagctgtat ggaaggagaa |
| 7201 | catgaatctc tcagaggccc aagtacaggc gttagcatta agtagacaag ccagccagga |
| 7261 | gcttgatgtt aaacgaagag aagcaatcta caatgatgta ttgacaaaac aacagatgtt |
| 7321 | aatcagctgt gttcagcgaa tacttatgaa cagaaggctc cagcagcagt acaatcagca |
| 7381 | gcaacagcaa caaatgactt atcaacaagc aacactgggt cacctcatga tgccaaagcc |
| 7441 | cccaaatttg atcatgaatc cttctaacta ccagcagatt gatatgagag gaatgtatca |
| 7501 | gccagtggct ggtggtatgc agccaccacc attacagcgt gcaccacccc caatgagaag |
| 7561 | caaaaatcca ggaccttccc aagggaaatc aatgtgattt tgcactaaaa gcttaatgga |
| 7621 | ttgttaaaat catagaaaga tcttttattt ttttaggaat caatgactta acagaactca |
| 7681 | actgtataaa tagtttggtc cccttaaatg ccaatcttcc atattagttt tacttttttt |
| 7741 | ttttttaaat agggcatacc atttcttcct gacatttgtc agtgatgttg cctagaatct |
| 7801 | tcttacacac gctgagtaca gaagatattt caaattgttt tcagtgaaaa caagtccttc |
| 7861 | cataatagta acaactccac agatttcctc tctaaatttt tatgcctgct tttagcaacc |
| 7921 | ataaaattgt cataaaatta ataaatttag gaaagaataa agatttatat attcattctt |
| 7981 | tacatataaa aacacacagc tgagttctta gagttgattc ctcaagttat gaaatacttt |
| 8041 | tgtacttaat ccatttcttg attaaagtga ttgaaatggt tttaatgttc ttttgactga |
| 8101 | agtctgaaac tgggctcctg ctttattgtc tctgtgactg aaagttagaa actgagggtt |
| 8161 | atctttgaca cagaattgtg tgcaatattc ttaaatacta ctgctctaaa agttggagaa |
| 8221 | gtcttgcagt tatcttagca ttgtataaac agccttaagt atagcctaag aagagaattc |
| 8281 | ctttttcttc tttagtcctt ctgccatttt ttattttcag ttatatgtgc tgaaataatt |
| 8341 | actggtaaaa tttcagggtt gtggattatc ttccacacat gaattttctc tctcctggca |
| 8401 | cgaatataaa gcacatctct taactgcatg gtgccagtgc taatgcttca tcctgttgct |
| 8461 | ggcagtggga tgtggactta gaaaatcaag ttctagcatt ttagtaggtt aacactgaag |
| 8521 | ttgtggttgt taggttcaca ccctgtttta taaacaacat caaaatggca gaaccattgc |
| 8581 | tgactttagg ttcacatgag gaatgtactt ttaacaattc ccagtactat cagtattgtg |
| 8641 | aaataattcc tctgaaagat aagaatcact ggcttctatg cgcttctttt ctctcatcat |
| 8701 | catgttcttt taccccagtt tccttacatt tttttaaatt gtttcagagt ttgttttttt |
| 8761 | tttagtttag attgtgaggc aattattaaa tcaaaattaa ttcatccaat acccctttac |
| 8821 | tagaagtttt actagaaaat gtattacatt ttattttttc ttaatccagt tctgcaaaaa |
| 8881 | tgacctataa atttattcat gtacaatttt ggttacttga attgttaaag aaaacattgt |
| 8941 | ttttgactat gggagtcaac tcaacatggc agaaccattt ttgagatgat gatacaacag |
| 9001 | gtagtgaaac agcttaagaa ttccaaaaaa aaaaaaaaaa aaaaaaaaaa gaaaactggg |
| 9061 | tttgggcttt gctttaggta tcactggatt agaatgagtt taacattagc taaaactgct |
| 9121 | ttgagttgtt tggatgatta agagattgcc atttttatct tggaagaact agtggtaaaa |
| 9181 | catccaagag cactaggatt gtgatacaga atttgtgagg tttggtggat ccacgcccct |
| 9241 | ctcccccact ttcccatgat gaaatatcac taataaatcc tgtatattta gatattatgc |
| 9301 | tagccatgta atcagattta tttaattggg tggggcaggt gtgtatttac tttagaaaaa |
| 9361 | atgaaaaaga caagatttat gagaaatatt tgaaggcagt acactctggc caactgttac |
| 9421 | cagttggtat ttctacaagt tcagaatatt ttaaacctga tttactagac ctgggaattt |
| 9481 | tcaacatggt ctaattattt actcaaagac atagatgtga aaattttagg caaccttcta |
| 9541 | aatctttttc accatggatg aaactataac ttaaagaata atacttagaa gggttaattg |
| 9601 | gaaatcagag tttgaaataa aacttggacc actttgtata cactcttctc acttgacatt |
| 9661 | ttagctatat aatatgtact ttgagtataa catcaagctt taacaaatat ttaaagacaa |
| 9721 | aaaaatcacg tcagtaaaat actaaaaggc tcatttttat atttgtttta gatgttttaa |
| 9781 | atagttgcaa tggattaaaa atgatgattt aaaatgttgc ttgtaataca gttttgcctg |
| 9841 | ctaaattctc cacattttgt aacctgtttt atttctttgg gtgtaaagcg tttttgctta |
| 9901 | gtattgtgat attgtatatg ttttgtccca gttgtatagt aatgtttcag tccatcatcc |
| 9961 | agctttggct gctgaaatca tacagctgtg aagacttgcc tttgtttctg ttagactgct |
| 10021 | tttcagttct gtattgagta tcttaagtac tgtagaaaag atgtcacttc ttcctttaag |
| 10081 | gctgttttgt aatatatata aggactggaa ttgtgttttt aaagaaaagc attcaagtat |
| 10141 | gacaatatac tatctgtgtt ttcaccattc aaagtgctgt ttagtagttg aaacttaaac |
| 10201 | tatttaatgt catttaataa agtgaccaaa atgtgttgtg ctctttattg tattttcaca |
| 10261 | gctttgaaaa tctgtgcaca tactgtttca tagaaaatgt atagcttttg ttgtcctata |
| 10321 | taatggtggt tcttttgcac atttagttat ttaatattga gaggtcacga agtttggtta |
| 10381 | ttgaatctgt tatatactaa attctgtaaa gggagatctc tcatctcaaa aagaatttac |
| 10441 | ataccaggaa gtccatgtgt gtttgtgtta gttttggatg tctttgtgta atccagcccc |
| 10501 | atttcctgtt tcccaacagc tgtaacactc attttaagtc aagcagggct accaacccac |
| 10561 | acttgataga aaagctgctt accattcaga agcttcctta ttacctggcc tccaaatgag |
| 10621 | ctgaatattt tgtagccttc ccttagctat gttcattttc cctccattat cataaaatca |
| 10681 | gatcgatatt tatgtgcccc aaacaaaact ttaagagcag ttacattctg tcccagtagc |
| 10741 | ccttgtttcc tttgagagta gcatgttgtg aggctataga gacttattct accagtaaaa |
| 10801 | caggtcaatc cttttacatg tttattatac taaaaattat gttcagggta tttactactt |
| 10861 | tatttcacca gactcagtct caagtgactt ggctatctcc aaatcagatc tacccttaga |
| 10921 | gaataaacat ttttctaccg ttattttttt tcaagtctat aatctgagcc agtcccaaag |
| 10981 | gagtgatcaa gtttcagaaa tgctttcatc ttcacaacat tttatatata ctattatatg |
| 11041 | gggtgaataa agttttaaat ccgaaatata aaaaaaaaaa aaaaaaaa |

A subject in need thereof may have reduced expression, haploinsufficiency, and/or loss of function of ARID1A. For example, a subject may comprise a mutation selected from the group consisting of a nonsense mutation for the wild type residue cysteine (C) at amino acid position 884 of SEQ ID NO: 11 (C884*), a substitution of lysine (K) for the wild type residue glutamic acid (E) at amino acid position 966 (E966K), a nonsense mutation for the wild type residue glutamine (Q) at amino acid position 1411 of SEQ ID NO: 11 (Q1411*), a frame shift mutation at the wild type residue phenylalanine (F) at amino acid position 1720 of SEQ ID NO: 11 (F1720fs), a frame shift mutation after the wild type residue glycine (G) at amino acid position 1847 of SEQ ID NO: 11 (G1847fs), a frame shift mutation at the wild type residue cysteine (C) at amino acid position 1874 of SEQ ID NO: 11 (C1874fs), a substitution of glutamic acid (E) for the wild type residue aspartic acid (D) at amino acid position 1957 (D1957E), a nonsense mutation for the wild type residue glutamine (Q) at amino acid position 1430 of SEQ ID NO: 11 (Q1430*), a frame shift mutation at the wild type residue arginine (R) at amino acid position 1721 of SEQ ID NO: 11 (R1721fs), a substitution of glutamic acid (E) for the wild type residue glycine (G) at amino acid position 1255 (G1255E), a frame shift mutation at the wild type residue glycine (G) at amino acid position 284 of SEQ ID NO: 11 (G284fs), a nonsense mutation for the wild type residue arginine (R) at amino acid position 1722 of SEQ ID NO: 11 (R1722*), a frame shift mutation at the wild type residue methionine (M) at amino acid position 274 of SEQ ID NO: 11 (M274fs), a frame shift mutation at the wild type residue glycine (G) at amino acid position 1847 of SEQ ID NO: 11 (G1847fs), a frame shift mutation at the wild type residue P at amino acid position 559 of SEQ ID NO: 11 (P559fs), a nonsense mutation for the wild type residue arginine (R) at amino acid position 1276 of SEQ ID NO: 11 (R1276*), a frame shift mutation at the wild type residue glutamine (Q) at amino acid position 2176 of SEQ ID NO: 11 (Q2176fs), a frame shift mutation at the wild type residue histidine (H) at amino acid position 203 of SEQ ID NO: 11 (H203fs), a frame shift mutation at the wild type residue alanine (A) at amino acid position 591 of SEQ ID NO: 11 (A591fs), a nonsense mutation for the wild type residue glutamine (Q) at amino acid position 1322 of SEQ ID NO: 11 (Q1322*), a nonsense mutation for the wild type residue serine (S) at amino acid position 2264 of SEQ ID NO: 11 (S2264*), a nonsense mutation for the wild type residue glutamine (Q) at amino acid position 586 of SEQ ID NO: 11 (Q586*), a frame shift mutation at the wild type residue glutamine (Q) at amino acid position 548 of SEQ ID NO: 11 (Q548fs), and a frame shift mutation at the wild type residue asparagine (N) at amino acid position 756 of SEQ ID NO: 11 (N756fs). “*” used herein refers to a stop codon. “fs” used herein refers to a frame shift.

| AT-rich interactive domain-containing protein 1A (ARID1A) isoform a [Homo sapiens] | |
|---|---|
| (SEQ ID NO: 9) | |
| 1 | maaqvapaaa sslgnppppp pselkkaeqq qreeaggeaa aaaaaergem kaaagqeseg |
| 61 | pavgppqplg kelqdgaesn gggggggags gggpgaepdl knsngnagpr palnnnltep |
| 121 | pggggggssd gvgapphsaa aalpppaygf gqpygrspsa vaaaaaavfh qqhggqqspg |
| 181 | laalqsgggg glepyagpqq nshdhgfpnh qynsyypnrs aypppapaya lssprggtpg |
| 241 | sgaaaaagsk pppsssasas sssssfaqqr fgamggggps aagggtpqpt atptlnqllt |
| 301 | spssargyqg ypggdysggp qdggagkgpa dmasqcwgaa aaaaaaaaas ggaqqrshha |
| 361 | pmspgssggg gqplartpqp sspmdqmgkm rpqpyggtnp ysqqqgppsg pqqghgypgq |
| 421 | pygsqtpqry pmtmqgraqs amgglsytqq ippygqqgps gygqqgqtpy ynqqsphpqq |
| 481 | qqppysqqpp sqtphaqpsy qqqpqsqppq lqssqppysq qpsqpphqqs papypsqqst |
| 541 | tqqhpqsqpp ysqpqaqspy qqqqpqqpap stlsqqaayp qpqsqqsqqt aysqqrfppp |
| 601 | qelsqdsfgs qassapsmts skggqedmnl slqsrpsslp dlsgsiddlp mgtegalspg |
| 661 | vstsgisssq geqsnpaqsp fsphtsphlp girgpspspv gspasvaqsr sgplspaavp |
| 721 | gnqmpprpps gqsdsimhps mnqssiaqdr gymqrnpqmp qysspqpgsa lsprqpsggq |
| 781 | ihtgmgsyqq nsmgsygpqg gqygpqggyp rqpnynalpn anypsagmag ginpmgaggq |
| 841 | mhgqpgippy gtlppgrmsh asmgnrpygp nmanmppqvg sgmcpppggm nrktqetava |
| 901 | mhvaansiqn rppgypnmnq ggmmgtgppy gqginsmagm inpqgppysm ggtmannsag |
| 961 | maaspemmgl gdvkltpatk mnnkadgtpk teskskksss stttnekitk lyelggeper |
| 1021 | kmwvdrylaf teekamgmtn lpavgrkpld lyrlyvsvke iggltqvnkn kkwrelatnl |
| 1081 | nvgtsssaas slkkqyiqcl yafeckierg edpppdifaa adskksqpki qppspagsgs |
| 1141 | mqgpqtpqst sssmaeggdl kpptpastph sqipplpgms rsnsvgiqda fndgsdstfq |
| 1201 | krnsmtpnpg yqpsmntsdm mgrmsyepnk dpygsmrkap gsdpfmssgq gpnggmgdpy |
| 1261 | sraagpglgn vamgprqhyp yggpydrvrt epgigpegnm stgapqpnlm psnpdsgmys |
| 1321 | psryppqqqq qqqqrhdsyg nqfstqgtps gspfpsqqtt myqqqqqnyk rpmdgtygpp |
| 1381 | akrhegemys vpystgqgqp qqqqlppaqp qpasqqqaaq pspqqdvynq ygnaypatat |
| 1441 | aaterrpagg pqnqfpfqfg rdrvsappgt naqqnmppqm mggpiqasae vaqqgtmwqg |
| 1501 | rndmtynyan rqstgsapqg payhgvnrtd emlhtdqran hegswpshgt rqppygpsap |
| 1561 | vppmtrppps nyqpppsmqn hipqvsspap lprpmenrts pskspflhsg mkmqkagppv |
| 1621 | pashiapapv qppmirrdit fppgsveatq pvlkqrrrlt mkdigtpeaw rvmmslksgl |
| 1681 | laestwaldt inillyddns imtfnlsqlp gllellveyf rrclieifgi lkeyevgdpg |
| 1741 | qrtlldpgrf skvsspapme ggeeeeellg pkleeeeeee vvendeeiaf sgkdkpasen |
| 1801 | seekliskfd klpvkivqkn dpfvvdcsdk lgrvqefdsg llhwrigggd ttehiqthfe |
| 1861 | sktellpsrp hapcppaprk hvttaegtpg ttdgegpppd gppekritat mddmlstrss |
| 1921 | tltedgakss eaikesskfp fgispaqshr nikiledeph skdetplctl ldwqdslakr |
| 1981 | cvcvsntirs lsfvpgndfe mskhpgllli lgklillhhk hperkqaplt yekeeeqdqg |
| 2041 | vscnkvewww dclemlrent lvtlanisgq ldlspypesi clpvldgllh wavcpsaeaq |
| 2101 | dpfstlgpna vlspqrlvle tlsklsiqdn nvdlilatpp fsrleklyst mvrflsdrkn |
| 2161 | pvcremavvl lanlaqgdsl aaraiavqkg signllgfle dslaatqfqq sqasllhmqn |
| 2221 | ppfeptsvdm mrraaralla lakvdenhse ftlyesrlld isysplmnsl vsqvicdvlf |
| 2281 | ligqs |
| Homo sapiens AT rich interactive domain 1A (SWI-like)(ARID1A), transcript variant 1, | |
| mRNA | |
| (SEQ ID NO: 10) | |
| 1 | cagaaagcgg agagtcacag cggggccagg ccctggggag cggagcctcc accgcccccc |
| 61 | tcattcccag gcaagggctt ggggggaatg agccgggaga gccgggtccc gagcctacag |
| 121 | agccgggagc agctgagccg ccggcgcctc ggccgccgcc gccgcctcct cctcctccgc |
| 181 | cgccgccagc ccggagcctg agccggcggg gcggggggga gaggagcgag cgcagcgcag |
| 241 | cagcggagcc ccgcgaggcc cgcccgggcg ggtggggagg gcagcccggg ggactgggcc |
| 301 | ccggggcggg gtgggagggg gggagaagac gaagacaggg ccgggtctct ccgcggacga |
| 361 | gacagcgggg atcatggccg cgcaggtcgc ccccgccgcc gccagcagcc tgggcaaccc |
| 421 | gccgccgccg ccgccctcgg agctgaagaa agccgagcag cagcagcggg aggaggcggg |
| 481 | gggcgaggcg gcggcggcgg cagcggccga gcgcggggaa atgaaggcag ccgccgggca |
| 541 | ggaaagcgag ggccccgccg tggggccgcc gcagccgctg ggaaaggagc tgcaggacgg |
| 601 | ggccgagagc aatgggggtg gcggcggcgg cggagccggc agcggcggcg ggcccggcgc |
| 661 | ggagccggac ctgaagaact cgaacgggaa cgcgggccct aggcccgccc tgaacaataa |
| 721 | cctcacggag ccgcccggcg gcggcggtgg cggcagcagc gatggggtgg gggcgcctcc |
| 781 | tcactcagcc gcggccgcct tgccgccccc agcctacggc ttcgggcaac cctacggccg |
| 841 | gagcccgtct gccgtcgccg ccgccgcggc cgccgtcttc caccaacaac atggcggaca |
| 901 | acaaagccct ggcctggcag cgctgcagag cggcggcggc gggggcctgg agccctacgc |
| 961 | ggggccccag cagaactctc acgaccacgg cttccccaac caccagtaca actcctacta |
| 1021 | ccccaaccgc agcgcctacc ccccgcccgc cccggcctac gcgctgagct ccccgagagg |
| 1081 | tggcactccg ggctccggcg cggcggcggc tgccggctcc aagccgcctc cctcctccag |
| 1141 | cgcctccgcc tcctcgtcgt cttcgtcctt cgctcagcag cgcttcgggg ccatgggggg |
| 1201 | aggcggcccc tccgcggccg gcgggggaac tccccagccc accgccaccc ccaccctcaa |
| 1261 | ccaactgctc acgtcgccca gctcggcccg gggctaccag ggctaccccg ggggcgacta |
| 1321 | cagtggcggg ccccaggacg ggggcgccgg caagggcccg gcggacatgg cctcgcagtg |
| 1381 | ttggggggct gcggcggcgg cagctgcggc ggcggccgcc tcgggagggg cccaacaaag |
| 1441 | gagccaccac gcgcccatga gccccgggag cagcggcggc ggggggcagc cgctcgcccg |
| 1501 | gacccctcag ccatccagtc caatggatca gatgggcaag atgagacctc agccatatgg |
| 1561 | cgggactaac ccatactcgc agcaacaggg acctccgtca ggaccgcagc aaggacatgg |
| 1621 | gtacccaggg cagccatacg ggtcccagac cccgcagcgg tacccgatga ccatgcaggg |
| 1681 | ccgggcgcag agtgccatgg gcggcctctc ttatacacag cagattcctc cttatggaca |
| 1741 | acaaggcccc agcgggtatg gtcaacaggg ccagactcca tattacaacc agcaaagtcc |
| 1801 | tcaccctcag cagcagcagc caccctactc ccagcaacca ccgtcccaga cccctcatgc |
| 1861 | ccaaccttcg tatcagcagc agccacagtc tcaaccacca cagctccagt cctctcagcc |
| 1921 | tccatactcc cagcagccat cccagcctcc acatcagcag tccccggctc catacccctc |
| 1981 | ccagcagtcg acgacacagc agcaccccca gagccagccc ccctactcac agccacaggc |
| 2041 | tcagtctcct taccagcagc agcaacctca gcagccagca ccctcgacgc tctcccagca |
| 2101 | ggctgcgtat cctcagcccc agtctcagca gtcccagcaa actgcctatt cccagcagcg |
| 2161 | cttccctcca ccgcaggagc tatctcaaga ttcatttggg tctcaggcat cctcagcccc |
| 2221 | ctcaatgacc tccagtaagg gagggcaaga agatatgaac ctgagccttc agtcaagacc |
| 2281 | ctccagcttg cctgatctat ctggttcaat agatgacctc cccatgggga cagaaggagc |
| 2341 | tctgagtcct ggagtgagca catcagggat ttccagcagc caaggagagc agagtaatcc |
| 2401 | agctcagtct cctttctctc ctcatacctc ccctcacctg cctggcatcc gaggcccttc |
| 2461 | cccgtcccct gttggctctc ccgccagtgt tgctcagtct cgctcaggac cactctcgcc |
| 2521 | tgctgcagtg ccaggcaacc agatgccacc tcggccaccc agtggccagt cggacagcat |
| 2581 | catgcatcct tccatgaacc aatcaagcat tgcccaagat cgaggttata tgcagaggaa |
| 2641 | cccccagatg ccccagtaca gttcccccca gcccggctca gccttatctc cgcgtcagcc |
| 2701 | ttccggagga cagatacaca caggcatggg ctcctaccag cagaactcca tggggagcta |
| 2761 | tggtccccag gggggtcagt atggcccaca aggtggctac cccaggcagc caaactataa |
| 2821 | tgccttgccc aatgccaact accccagtgc aggcatggct ggaggcataa accccatggg |
| 2881 | tgccggaggt caaatgcatg gacagcctgg catcccacct tatggcacac tccctccagg |
| 2941 | gaggatgagt cacgcctcca tgggcaaccg gccttatggc cctaacatgg ccaatatgcc |
| 3001 | acctcaggtt gggtcaggga tgtgtccccc accagggggc atgaaccgga aaacccaaga |
| 3061 | aactgctgtc gccatgcatg ttgctgccaa ctctatccaa aacaggccgc caggctaccc |
| 3121 | caatatgaat caagggggca tgatgggaac tggacctcct tatggacaag ggattaatag |
| 3181 | tatggctggc atgatcaacc ctcagggacc cccatattcc atgggtggaa ccatggccaa |
| 3241 | caattctgca gggatggcag ccagcccaga gatgatgggc cttggggatg taaagttaac |
| 3301 | tccagccacc aaaatgaaca acaaggcaga tgggacaccc aagacagaat ccaaatccaa |
| 3361 | gaaatccagt tcttctacta caaccaatga gaagatcacc aagttgtatg agctgggtgg |
| 3421 | tgagcctgag aggaagatgt gggtggaccg ttatctggcc ttcactgagg agaaggccat |
| 3481 | gggcatgaca aatctgcctg ctgtgggtag gaaacctctg gacctctatc gcctctatgt |
| 3541 | gtctgtgaag gagattggtg gattgactca ggtcaacaag aacaaaaaat ggcgggaact |
| 3601 | tgcaaccaac ctcaatgtgg gcacatcaag cagtgctgcc agctccttga aaaagcagta |
| 3661 | tatccagtgt ctctatgcct ttgaatgcaa gattgaacgg ggagaagacc ctcccccaga |
| 3721 | catctttgca gctgctgatt ccaagaagtc ccagcccaag atccagcctc cctctcctgc |
| 3781 | gggatcagga tctatgcagg ggccccagac tccccagtca accagcagtt ccatggcaga |
| 3841 | aggaggagac ttaaagccac caactccagc atccacacca cacagtcaga tccccccatt |
| 3901 | gccaggcatg agcaggagca attcagttgg gatccaggat gcctttaatg atggaagtga |
| 3961 | ctccacattc cagaagcgga attccatgac tccaaaccct gggtatcagc ccagtatgaa |
| 4021 | tacctctgac atgatggggc gcatgtccta tgagccaaat aaggatcctt atggcagcat |
| 4081 | gaggaaagct ccagggagtg atcccttcat gtcctcaggg cagggcccca acggcgggat |
| 4141 | gggtgacccc tacagtcgtg ctgccggccc tgggctagga aatgtggcga tgggaccacg |
| 4201 | acagcactat ccctatggag gtccttatga cagagtgagg acggagcctg gaatagggcc |
| 4261 | tgagggaaac atgagcactg gggccccaca gccgaatctc atgccttcca acccagactc |
| 4321 | ggggatgtat tctcctagcc gctacccccc gcagcagcag cagcagcagc agcaacgaca |
| 4381 | tgattcctat ggcaatcagt tctccaccca aggcacccct tctggcagcc ccttccccag |
| 4441 | ccagcagact acaatgtatc aacagcaaca gcagaattac aagcggccaa tggatggcac |
| 4501 | atatggccct cctgccaagc ggcacgaagg ggagatgtac agcgtgccat acagcactgg |
| 4561 | gcaggggcag cctcagcagc agcagttgcc cccagcccag ccccagcctg ccagccagca |
| 4621 | acaagctgcc cagccttccc ctcagcaaga tgtatacaac cagtatggca atgcctatcc |
| 4681 | tgccactgcc acagctgcta ctgagcgccg accagcaggc ggcccccaga accaatttcc |
| 4741 | attccagttt ggccgagacc gtgtctctgc accccctggc accaatgccc agcaaaacat |
| 4801 | gccaccacaa atgatgggcg gccccataca ggcatcagct gaggttgctc agcaaggcac |
| 4861 | catgtggcag gggcgtaatg acatgaccta taattatgcc aacaggcaga gcacgggctc |
| 4921 | tgccccccag ggccccgcct atcatggcgt gaaccgaaca gatgaaatgc tgcacacaga |
| 4981 | tcagagggcc aaccacgaag gctcgtggcc ttcccatggc acacgccagc ccccatatgg |
| 5041 | tccctctgcc cctgtgcccc ccatgacaag gccccctcca tctaactacc agcccccacc |
| 5101 | aagcatgcag aatcacattc ctcaggtatc cagccctgct cccctgcccc ggccaatgga |
| 5161 | gaaccgcacc tctcctagca agtctccatt cctgcactct gggatgaaaa tgcagaaggc |
| 5221 | aggtccccca gtacctgcct cgcacatagc acctgcccct gtgcagcccc ccatgattcg |
| 5281 | gcgggatatc accttcccac ctggctctgt tgaagccaca cagcctgtgt tgaagcagag |
| 5341 | gaggcggctc acaatgaaag acattggaac cccggaggca tggcgggtaa tgatgtccct |
| 5401 | caagtctggt ctcctggcag agagcacatg ggcattagat accatcaaca tcctgctgta |
| 5461 | tgatgacaac agcatcatga ccttcaacct cagtcagctc ccagggttgc tagagctcct |
| 5521 | tgtagaatat ttccgacgat gcctgattga gatctttggc attttaaagg agtatgaggt |
| 5581 | gggtgaccca ggacagagaa cgctactgga tcctgggagg ttcagcaagg tgtctagtcc |
| 5641 | agctcccatg gagggtgggg aagaagaaga agaacttcta ggtcctaaac tagaagagga |
| 5701 | agaagaagag gaagtagttg aaaatgatga ggagatagcc ttttcaggca aggacaagcc |
| 5761 | agcttcagag aatagtgagg agaagctgat cagtaagttt gacaagcttc cagtaaagat |
| 5821 | cgtacagaag aatgatccat ttgtggtgga ctgctcagat aagcttgggc gtgtgcagga |
| 5881 | gtttgacagt ggcctgctgc actggcggat tggtgggggg gacaccactg agcatatcca |
| 5941 | gacccacttc gagagcaaga cagagctgct gccttcccgg cctcacgcac cctgcccacc |
| 6001 | agcccctcgg aagcatgtga caacagcaga gggtacacca gggacaacag accaggaggg |
| 6061 | gcccccacct gatggacctc cagaaaaacg gatcacagcc actatggatg acatgttgtc |
| 6121 | tactcggtct agcaccttga ccgaggatgg agctaagagt tcagaggcca tcaaggagag |
| 6181 | cagcaagttt ccatttggca ttagcccagc acagagccac cggaacatca agatcctaga |
| 6241 | ggacgaaccc cacagtaagg atgagacccc actgtgtacc cttctggact ggcaggattc |
| 6301 | tcttgccaag cgctgcgtct gtgtgtccaa taccattcga agcctgtcat ttgtgccagg |
| 6361 | caatgacttt gagatgtcca aacacccagg gctgctgctc atcctgggca agctgatcct |
| 6421 | gctgcaccac aagcacccag aacggaagca ggcaccacta acttatgaaa aggaggagga |
| 6481 | acaggaccaa ggggtgagct gcaacaaagt ggagtggtgg tgggactgct tggagatgct |
| 6541 | ccgggaaaac accttggtta cactcgccaa catctcgggg cagttggacc tatctccata |
| 6601 | ccccgagagc atttgcctgc ctgtcctgga cggactccta cactgggcag tttgcccttc |
| 6661 | agctgaagcc caggacccct tttccaccct gggccccaat gccgtccttt ccccgcagag |
| 6721 | actggtcttg gaaaccctca gcaaactcag catccaggac aacaatgtgg acctgattct |
| 6781 | ggccacaccc cccttcagcc gcctggagaa gttgtatagc actatggtgc gcttcctcag |
| 6841 | tgaccgaaag aacccggtgt gccgggagat ggctgtggta ctgctggcca acctggctca |
| 6901 | gggggacagc ctggcagctc gtgccattgc agtgcagaag ggcagtatcg gcaacctcct |
| 6961 | gggcttccta gaggacagcc ttgccgccac acagttccag cagagccagg ccagcctcct |
| 7021 | ccacatgcag aacccaccct ttgagccaac tagtgtggac atgatgcggc gggctgcccg |
| 7081 | cgcgctgctt gccttggcca aggtggacga gaaccactca gagtttactc tgtacgaatc |
| 7141 | acggctgttg gacatctcgg tatcaccgtt gatgaactca ttggtttcac aagtcatttg |
| 7201 | tgatgtactg tttttgattg gccagtcatg acagccgtgg gacacctccc ccccccgtgt |
| 7261 | gtgtgtgcgt gtgtggagaa cttagaaact gactgttgcc ctttatttat gcaaaaccac |
| 7321 | ctcagaatcc agtttaccct gtgctgtcca gcttctccct tgggaaaaag tctctcctgt |
| 7381 | ttctctctcc tccttccacc tcccctccct ccatcacctc acgcctttct gttccttgtc |
| 7441 | ctcaccttac tcccctcagg accctacccc accctctttg aaaagacaaa gctctgccta |
| 7501 | catagaagac tttttttatt ttaaccaaag ttactgttgt ttacagtgag tttggggaaa |
| 7561 | aaaaataaaa taaaaatggc tttcccagtc cttgcatcaa cgggatgcca catttcataa |
| 7621 | ctgtttttaa tggtaaaaaa aaaaaaaaaa aatacaaaaa aaaattctga aggacaaaaa |
| 7681 | aggtgactgc tgaactgtgt gtggtttatt gttgtacatt cacaatcttg caggagccaa |
| 7741 | gaagttcgca gttgtgaaca gaccctgttc actggagagg cctgtgcagt agagtgtaga |
| 7801 | ccctttcatg tactgtactg tacacctgat actgtaaaca tactgtaata ataatgtctc |
| 7861 | acatggaaac agaaaacgct gggtcagcag caagctgtag tttttaaaaa tgtttttagt |
| 7921 | taaacgttga ggagaaaaaa aaaaaaggct tttcccccaa agtatcatgt gtgaacctac |
| 7981 | aacaccctga cctctttctc tcctccttga ttgtatgaat aaccctgaga tcacctctta |
| 8041 | gaactggttt taacctttag ctgcagcggc tacgctgcca cgtgtgtata tatatgacgt |
| 8101 | tgtacattgc acataccctt ggatccccac agtttggtcc tcctcccagc taccccttta |
| 8161 | tagtatgacg agttaacaag ttggtgacct gcacaaagcg agacacagct atttaatctc |
| 8221 | ttgccagata tcgcccctct tggtgcgatg ctgtacaggt ctctgtaaaa agtccttgct |
| 8281 | gtctcagcag ccaatcaact tatagtttat ttttttctgg gtttttgttt tgttttgttt |
| 8341 | tctttctaat cgaggtgtga aaaagttcta ggttcagttg aagttctgat gaagaaacac |
| 8401 | aattgagatt ttttcagtga taaaatctgc atatttgtat ttcaacaatg tagctaaaac |
| 8461 | ttgatgtaaa ttcctccttt ttttcctttt ttggcttaat gaatatcatt tattcagtat |
| 8521 | gaaatcttta tactatatgt tccacgtgtt aagaataaat gtacattaaa tcttggtaag |
| 8581 | acttt |
| AT-rich interactive domain-containing protein 1A (ARID1A) isoform b | |
| (SEQ ID NO: 11) | |
| 1 | maaqvapaaa sslgnppppp pselkkaeqq qreeaggeaa aaaaaergem kaaagqeseg |
| 61 | pavgppqplg kelqdgaesn gggggggags gggpgaepdl knsngnagpr palnnnltep |
| 121 | pggggggssd gvgapphsaa aalpppaygf gqpygrspsa vaaaaaavfh qqhggqqspg |
| 181 | laalqsgggg glepyagpqq nshdhgfpnh qynsyypnrs aypppapaya lssprggtpg |
| 241 | sgaaaaagsk pppsssasas sssssfaqqr fgamggggps aagggtpqpt atptlnqllt |
| 301 | spssargyqg ypggdysggp qdggagkgpa dmasqcwgaa aaaaaaaaas ggaqqrshha |
| 361 | pmspgssggg gqplartpqp sspmdqmgkm rpqpyggtnp ysqqqgppsg pqqghgypgq |
| 421 | pygsqtpqry pmtmqgraqs amgglsytqq ippygqqgps gygqqgqtpy ynqqsphpqq |
| 481 | qqppysqqpp sqtphaqpsy qqqpqsqppq lqssqppysq qpsqpphqqs papypsqqst |
| 541 | tqqhpqsqpp ysqpqaqspy qqqqpqqpap stlsqqaayp qpqsqqsqqt aysqqrfppp |
| 601 | qelsqdsfgs qassapsmts skggqedmnl slqsrpsslp dlsgsiddlp mgtegalspg |
| 661 | vstsgisssq geqsnpaqsp fsphtsphlp girgpspspv gspasvaqsr sgplspaavp |
| 721 | gnqmpprpps gqsdsimhps mnqssiaqdr gymqrnpqmp qysspqpgsa lsprqpsggq |
| 781 | ihtgmgsyqq nsmgsygpqg gqygpqggyp rqpnynalpn anypsagmag ginpmgaggq |
| 841 | mhgqpgippy gtlppgrmsh asmgnrpygp nmanmppqvg sgmcpppggm nrktqetava |
| 901 | mhvaansiqn rppgypnmnq ggmmgtgppy gqginsmagm inpqgppysm ggtmannsag |
| 961 | maaspemmgl gdvkltpatk mnnkadgtpk teskskksss stttnekitk lyelggeper |
| 1021 | kmwvdrylaf teekamgmtn lpavgrkpld lyrlyvsvke iggltqvnkn kkwrelatnl |
| 1081 | nvgtsssaas slkkqyiqcl yafeckierg edpppdifaa adskksqpki qppspagsgs |
| 1141 | mqgpqtpqst sssmaeggdl kpptpastph sqipplpgms rsnsvgiqda fndgsdstfq |
| 1201 | krnsmtpnpg yqpsmntsdm mgrmsyepnk dpygsmrkap gsdpfmssgq gpnggmgdpy |
| 1261 | sraagpglgn vamgprqhyp yggpydrvrt epgigpegnm stgapqpnlm psnpdsgmys |
| 1321 | psryppqqqq qqqqrhdsyg nqfstqgtps gspfpsqqtt myqqqqqvss paplprpmen |
| 1381 | rtspskspfl hsgmkmqkag ppvpashiap apvqppmirr ditfppgsve atqpvlkqrr |
| 1441 | rltmkdigtp eawrvmmslk sgllaestwa ldtinillyd dnsimtfnls qlpgllellv |
| 1501 | eyfrrcliei fgilkeyevg dpgqrtlldp grfskvsspa pmeggeeeee llgpkleeee |
| 1561 | eeevvendee iafsgkdkpa senseeklis kfdklpvkiv qkndpfvvdc sdklgrvqef |
| 1621 | dsgllhwrig ggdttehiqt hfesktellp srphapcppa prkhvttaeg tpgttdqegp |
| 1681 | ppdgppekri tatmddmlst rsstltedga ksseaikess kfpfgispaq shrnikiled |
| 1741 | ephskdetpl ctlldwqdsl akrcvcvsnt irslsfvpgn dfemskhpgl llilgklill |
| 1801 | hhkhperkqa pltyekeeeq dqgvscnkve wwwdclemlr entlvtlani sgqldlspyp |
| 1861 | esiclpvldg llhwavcpsa eaqdpfstlg pnavlspqrl vletlsklsi qdnnvdlila |
| 1921 | tppfsrlekl ystmvrflsd rknpvcrema vvllanlaqg dslaaraiav qkgsignllg |
| 1981 | fledslaatq fqqsqasllh mqnppfepts vdmmrraara llalakvden hseftlyesr |
| 2041 | lldisvsplm nslvsqvicd vlfligqs |
| Homo sapiens AT rich interactive domain 1A (SWI-like)(ARID1A), transcript variant 2, | |
| mRNA | |
| (SEQ ID NO: 12) | |
| 1 | cagaaagcgg agagtcacag cggggccagg ccctggggag cggagcctcc accgcccccc |
| 61 | tcattcccag gcaagggctt ggggggaatg agccgggaga gccgggtccc gagcctacag |
| 121 | agccgggagc agctgagccg ccggcgcctc ggccgccgcc gccgcctcct cctcctccgc |
| 181 | cgccgccagc ccggagcctg agccggcggg gcggggggga gaggagcgag cgcagcgcag |
| 241 | cagcggagcc ccgcgaggcc cgcccgggcg ggtggggagg gcagcccggg ggactgggcc |
| 301 | ccggggcggg gtgggagggg gggagaagac gaagacaggg ccgggtctct ccgcggacga |
| 361 | gacagcgggg atcatggccg cgcaggtcgc ccccgccgcc gccagcagcc tgggcaaccc |
| 421 | gccgccgccg ccgccctcgg agctgaagaa agccgagcag cagcagcggg aggaggcggg |
| 481 | gggcgaggcg gcggcggcgg cagcggccga gcgcggggaa atgaaggcag ccgccgggca |
| 541 | ggaaagcgag ggccccgccg tggggccgcc gcagccgctg ggaaaggagc tgcaggacgg |
| 601 | ggccgagagc aatgggggtg gcggcggcgg cggagccggc agcggcggcg ggcccggcgc |
| 661 | ggagccggac ctgaagaact cgaacgggaa cgcgggccct aggcccgccc tgaacaataa |
| 721 | cctcacggag ccgcccggcg gcggcggtgg cggcagcagc gatggggtgg gggcgcctcc |
| 781 | tcactcagcc gcggccgcct tgccgccccc agcctacggc ttcgggcaac cctacggccg |
| 841 | gagcccgtct gccgtcgccg ccgccgcggc cgccgtcttc caccaacaac atggcggaca |
| 901 | acaaagccct ggcctggcag cgctgcagag cggcggcggc gggggcctgg agccctacgc |
| 961 | ggggccccag cagaactctc acgaccacgg cttccccaac caccagtaca actcctacta |
| 1021 | ccccaaccgc agcgcctacc ccccgcccgc cccggcctac gcgctgagct ccccgagagg |
| 1081 | tggcactccg ggctccggcg cggcggcggc tgccggctcc aagccgcctc cctcctccag |
| 1141 | cgcctccgcc tcctcgtcgt cttcgtcctt cgctcagcag cgcttcgggg ccatgggggg |
| 1201 | aggcggcccc tccgcggccg gcgggggaac tccccagccc accgccaccc ccaccctcaa |
| 1261 | ccaactgctc acgtcgccca gctcggcccg gggctaccag ggctaccccg ggggcgacta |
| 1321 | cagtggcggg ccccaggacg ggggcgccgg caagggcccg gcggacatgg cctcgcagtg |
| 1381 | ttggggggct gcggcggcgg cagctgcggc ggcggccgcc tcgggagggg cccaacaaag |
| 1441 | gagccaccac gcgcccatga gccccgggag cagcggcggc ggggggcagc cgctcgcccg |
| 1501 | gacccctcag ccatccagtc caatggatca gatgggcaag atgagacctc agccatatgg |
| 1561 | cgggactaac ccatactcgc agcaacaggg acctccgtca ggaccgcagc aaggacatgg |
| 1621 | gtacccaggg cagccatacg ggtcccagac cccgcagcgg tacccgatga ccatgcaggg |
| 1681 | ccgggcgcag agtgccatgg gcggcctctc ttatacacag cagattcctc cttatggaca |
| 1741 | acaaggcccc agcgggtatg gtcaacaggg ccagactcca tattacaacc agcaaagtcc |
| 1801 | tcaccctcag cagcagcagc caccctactc ccagcaacca ccgtcccaga cccctcatgc |
| 1861 | ccaaccttcg tatcagcagc agccacagtc tcaaccacca cagctccagt cctctcagcc |
| 1921 | tccatactcc cagcagccat cccagcctcc acatcagcag tccccggctc catacccctc |
| 1981 | ccagcagtcg acgacacagc agcaccccca gagccagccc ccctactcac agccacaggc |
| 2041 | tcagtctcct taccagcagc agcaacctca gcagccagca ccctcgacgc tctcccagca |
| 2101 | ggctgcgtat cctcagcccc agtctcagca gtcccagcaa actgcctatt cccagcagcg |
| 2161 | cttccctcca ccgcaggagc tatctcaaga ttcatttggg tctcaggcat cctcagcccc |
| 2221 | ctcaatgacc tccagtaagg gagggcaaga agatatgaac ctgagccttc agtcaagacc |
| 2281 | ctccagcttg cctgatctat ctggttcaat agatgacctc cccatgggga cagaaggagc |
| 2341 | tctgagtcct ggagtgagca catcagggat ttccagcagc caaggagagc agagtaatcc |
| 2401 | agctcagtct cctttctctc ctcatacctc ccctcacctg cctggcatcc gaggcccttc |
| 2461 | cccgtcccct gttggctctc ccgccagtgt tgctcagtct cgctcaggac cactctcgcc |
| 2521 | tgctgcagtg ccaggcaacc agatgccacc tcggccaccc agtggccagt cggacagcat |
| 2581 | catgcatcct tccatgaacc aatcaagcat tgcccaagat cgaggttata tgcagaggaa |
| 2641 | cccccagatg ccccagtaca gttcccccca gcccggctca gccttatctc cgcgtcagcc |
| 2701 | ttccggagga cagatacaca caggcatggg ctcctaccag cagaactcca tggggagcta |
| 2761 | tggtccccag gggggtcagt atggcccaca aggtggctac cccaggcagc caaactataa |
| 2821 | tgccttgccc aatgccaact accccagtgc aggcatggct ggaggcataa accccatggg |
| 2881 | tgccggaggt caaatgcatg gacagcctgg catcccacct tatggcacac tccctccagg |
| 2941 | gaggatgagt cacgcctcca tgggcaaccg gccttatggc cctaacatgg ccaatatgcc |
| 3001 | acctcaggtt gggtcaggga tgtgtccccc accagggggc atgaaccgga aaacccaaga |
| 3061 | aactgctgtc gccatgcatg ttgctgccaa ctctatccaa aacaggccgc caggctaccc |
| 3121 | caatatgaat caagggggca tgatgggaac tggacctcct tatggacaag ggattaatag |
| 3181 | tatggctggc atgatcaacc ctcagggacc cccatattcc atgggtggaa ccatggccaa |
| 3241 | caattctgca gggatggcag ccagcccaga gatgatgggc cttggggatg taaagttaac |
| 3301 | tccagccacc aaaatgaaca acaaggcaga tgggacaccc aagacagaat ccaaatccaa |
| 3361 | gaaatccagt tcttctacta caaccaatga gaagatcacc aagttgtatg agctgggtgg |
| 3421 | tgagcctgag aggaagatgt gggtggaccg ttatctggcc ttcactgagg agaaggccat |
| 3481 | gggcatgaca aatctgcctg ctgtgggtag gaaacctctg gacctctatc gcctctatgt |
| 3541 | gtctgtgaag gagattggtg gattgactca ggtcaacaag aacaaaaaat ggcgggaact |
| 3601 | tgcaaccaac ctcaatgtgg gcacatcaag cagtgctgcc agctccttga aaaagcagta |
| 3661 | tatccagtgt ctctatgcct ttgaatgcaa gattgaacgg ggagaagacc ctcccccaga |
| 3721 | catctttgca gctgctgatt ccaagaagtc ccagcccaag atccagcctc cctctcctgc |
| 3781 | gggatcagga tctatgcagg ggccccagac tccccagtca accagcagtt ccatggcaga |
| 3841 | aggaggagac ttaaagccac caactccagc atccacacca cacagtcaga tccccccatt |
| 3901 | gccaggcatg agcaggagca attcagttgg gatccaggat gcctttaatg atggaagtga |
| 3961 | ctccacattc cagaagcgga attccatgac tccaaaccct gggtatcagc ccagtatgaa |
| 4021 | tacctctgac atgatggggc gcatgtccta tgagccaaat aaggatcctt atggcagcat |
| 4081 | gaggaaagct ccagggagtg atcccttcat gtcctcaggg cagggcccca acggcgggat |
| 4141 | gggtgacccc tacagtcgtg ctgccggccc tgggctagga aatgtggcga tgggaccacg |
| 4201 | acagcactat ccctatggag gtccttatga cagagtgagg acggagcctg gaatagggcc |
| 4261 | tgagggaaac atgagcactg gggccccaca gccgaatctc atgccttcca acccagactc |
| 4321 | ggggatgtat tctcctagcc gctacccccc gcagcagcag cagcagcagc agcaacgaca |
| 4381 | tgattcctat ggcaatcagt tctccaccca aggcacccct tctggcagcc ccttccccag |
| 4441 | ccagcagact acaatgtatc aacagcaaca gcaggtatcc agccctgctc ccctgccccg |
| 4501 | gccaatggag aaccgcacct ctcctagcaa gtctccattc ctgcactctg ggatgaaaat |
| 4561 | gcagaaggca ggtcccccag tacctgcctc gcacatagca cctgcccctg tgcagccccc |
| 4621 | catgattcgg cgggatatca ccttcccacc tggctctgtt gaagccacac agcctgtgtt |
| 4681 | gaagcagagg aggcggctca caatgaaaga cattggaacc ccggaggcat ggcgggtaat |
| 4741 | gatgtccctc aagtctggtc tcctggcaga gagcacatgg gcattagata ccatcaacat |
| 4801 | cctgctgtat gatgacaaca gcatcatgac cttcaacctc agtcagctcc cagggttgct |
| 4861 | agagctcctt gtagaatatt tccgacgatg cctgattgag atctttggca ttttaaagga |
| 4921 | gtatgaggtg ggtgacccag gacagagaac gctactggat cctgggaggt tcagcaaggt |
| 4981 | gtctagtcca gctcccatgg agggtgggga agaagaagaa gaacttctag gtcctaaact |
| 5041 | agaagaggaa gaagaagagg aagtagttga aaatgatgag gagatagcct tttcaggcaa |
| 5101 | ggacaagcca gcttcagaga atagtgagga gaagctgatc agtaagtttg acaagcttcc |
| 5161 | agtaaagatc gtacagaaga atgatccatt tgtggtggac tgctcagata agcttgggcg |
| 5221 | tgtgcaggag tttgacagtg gcctgctgca ctggcggatt ggtggggggg acaccactga |
| 5281 | gcatatccag acccacttcg agagcaagac agagctgctg ccttcccggc ctcacgcacc |
| 5341 | ctgcccacca gcccctcgga agcatgtgac aacagcagag ggtacaccag ggacaacaga |
| 5401 | ccaggagggg cccccacctg atggacctcc agaaaaacgg atcacagcca ctatggatga |
| 5461 | catgttgtct actcggtcta gcaccttgac cgaggatgga gctaagagtt cagaggccat |
| 5521 | caaggagagc agcaagtttc catttggcat tagcccagca cagagccacc ggaacatcaa |
| 5581 | gatcctagag gacgaacccc acagtaagga tgagacccca ctgtgtaccc ttctggactg |
| 5641 | gcaggattct cttgccaagc gctgcgtctg tgtgtccaat accattcgaa gcctgtcatt |
| 5701 | tgtgccaggc aatgactttg agatgtccaa acacccaggg ctgctgctca tcctgggcaa |
| 5761 | gctgatcctg ctgcaccaca agcacccaga acggaagcag gcaccactaa cttatgaaaa |
| 5821 | ggaggaggaa caggaccaag gggtgagctg caacaaagtg gagtggtggt gggactgctt |
| 5881 | ggagatgctc cgggaaaaca ccttggttac actcgccaac atctcggggc agttggacct |
| 5941 | atctccatac cccgagagca tttgcctgcc tgtcctggac ggactcctac actgggcagt |
| 6001 | ttgcccttca gctgaagccc aggacccctt ttccaccctg ggccccaatg ccgtcctttc |
| 6061 | cccgcagaga ctggtcttgg aaaccctcag caaactcagc atccaggaca acaatgtgga |
| 6121 | cctgattctg gccacacccc ccttcagccg cctggagaag ttgtatagca ctatggtgcg |
| 6181 | cttcctcagt gaccgaaaga acccggtgtg ccgggagatg gctgtggtac tgctggccaa |
| 6241 | cctggctcag ggggacagcc tggcagctcg tgccattgca gtgcagaagg gcagtatcgg |
| 6301 | caacctcctg ggcttcctag aggacagcct tgccgccaca cagttccagc agagccaggc |
| 6361 | cagcctcctc cacatgcaga acccaccctt tgagccaact agtgtggaca tgatgcggcg |
| 6421 | ggctgcccgc gcgctgcttg ccttggccaa ggtggacgag aaccactcag agtttactct |
| 6481 | gtacgaatca cggctgttgg acatctcggt atcaccgttg atgaactcat tggtttcaca |
| 6541 | agtcatttgt gatgtactgt ttttgattgg ccagtcatga cagccgtggg acacctcccc |
| 6601 | cccccgtgtg tgtgtgcgtg tgtggagaac ttagaaactg actgttgccc tttatttatg |
| 6661 | caaaaccacc tcagaatcca gtttaccctg tgctgtccag cttctccctt gggaaaaagt |
| 6721 | ctctcctgtt tctctctcct ccttccacct cccctccctc catcacctca cgcctttctg |
| 6781 | ttccttgtcc tcaccttact cccctcagga ccctacccca ccctctttga aaagacaaag |
| 6841 | ctctgcctac atagaagact ttttttattt taaccaaagt tactgttgtt tacagtgagt |
| 6901 | ttggggaaaa aaaataaaat aaaaatggct ttcccagtcc ttgcatcaac gggatgccac |
| 6961 | atttcataac tgtttttaat ggtaaaaaaa aaaaaaaaaa atacaaaaaa aaattctgaa |
| 7021 | ggacaaaaaa ggtgactgct gaactgtgtg tggtttattg ttgtacattc acaatcttgc |
| 7081 | aggagccaag aagttcgcag ttgtgaacag accctgttca ctggagaggc ctgtgcagta |
| 7141 | gagtgtagac cctttcatgt actgtactgt acacctgata ctgtaaacat actgtaataa |
| 7201 | taatgtctca catggaaaca gaaaacgctg ggtcagcagc aagctgtagt ttttaaaaat |
| 7261 | gtttttagtt aaacgttgag gagaaaaaaa aaaaaggctt ttcccccaaa gtatcatgtg |
| 7321 | tgaacctaca acaccctgac ctctttctct cctccttgat tgtatgaata accctgagat |
| 7381 | cacctcttag aactggtttt aacctttagc tgcagcggct acgctgccac gtgtgtatat |
| 7441 | atatgacgtt gtacattgca catacccttg gatccccaca gtttggtcct cctcccagct |
| 7501 | acccctttat agtatgacga gttaacaagt tggtgacctg cacaaagcga gacacagcta |
| 7561 | tttaatctct tgccagatat cgcccctctt ggtgcgatgc tgtacaggtc tctgtaaaaa |
| 7621 | gtccttgctg tctcagcagc caatcaactt atagtttatt tttttctggg tttttgtttt |
| 7681 | gttttgtttt ctttctaatc gaggtgtgaa aaagttctag gttcagttga agttctgatg |
| 7741 | aagaaacaca attgagattt tttcagtgat aaaatctgca tatttgtatt tcaacaatgt |
| 7801 | agctaaaact tgatgtaaat tcctcctttt tttccttttt tggcttaatg aatatcattt |
| 7861 | attcagtatg aaatctttat actatatgtt ccacgtgtta agaataaatg tacattaaat |
| 7921 | cttggtaaga cttt |

The term “inducing differentiation” used herein refers to causing an immature or stem-like cell to develop into a more differentiated or terminally differentiated cell.

According to the methods of the disclosure, a “normal” cell may be used as a basis of comparison for one or more characteristics of a cancer cell, including expression and/or function of SNF5, ATRX, and/or ARID1A. As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell expresses a comparable amount of EZH2 as a cancer cell. Preferably a normal cell contains a wild type sequence for a SNF5, ATRX, and/or ARID1A gene, expresses a SNF5, ATRX, and/or ARID1A transcript without mutations, and expresses a SNF5, ATRX, and/or ARID1A protein without mutations that retains all functions a normal activity levels.

As used herein, “contacting a cell” refers to a condition in which a compound or other composition of matter is in direct contact with a cell, or is close enough to induce a desired biological effect in a cell.

As used herein, “treating” or “treat” describes the management and care of a subject for the purpose of combating a disease, condition, or disorder and includes the administration of an EZH2 inhibitor of the disclosure, or a pharmaceutically acceptable salt, prodrug, metabolite, polymorph or solvate thereof, to alleviate the symptoms or complications of cancer or to eliminate the cancer.

As used herein, the term “alleviate” is meant to describe a process by which the severity of a sign or symptom of cancer is decreased. Importantly, a sign or symptom can be alleviated without being eliminated. In a preferred embodiment, the administration of pharmaceutical compositions of the disclosure leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

In another aspect of the disclosure, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

As a cancer grows, it begins to push on nearby organs, blood vessels, and nerves. This pressure creates some of the signs and symptoms of cancer. Cancers may form in places where it does not cause any symptoms until the cancer has grown quite large.

Cancer may also cause symptoms such as fever, fatigue, or weight loss. This may be because cancer cells use up much of the body's energy supply or release substances that change the body's metabolism. Or the cancer may cause the immune system to react in ways that produce these symptoms. While the signs and symptoms listed above are the more common ones seen with cancer, there are many others that are less common and are not listed here. However, all art-recognized signs and symptoms of cancer are contemplated and encompassed by the disclosure.

Treating cancer may result in a reduction in size of a tumor. A reduction in size of a tumor may also be referred to as “tumor regression”. Preferably, after treatment according to the methods of the disclosure, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer may result in a reduction in tumor volume. Preferably, after treatment according to the methods of the disclosure, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer may result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer may result in a decrease in number of metastatic lesions in other tissues or organs distant from the primary tumor site. Preferably, after treatment according to the methods of the disclosure, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

An effective amount of an EZH2 inhibitor of the disclosure, or a pharmaceutically acceptable salt, prodrug, metabolite, polymorph or solvate thereof, is not significantly cytotoxic to normal cells. For example, a therapeutically effective amount of an EZH2 inhibitor of the disclosure is not significantly cytotoxic to normal cells if administration of the EZH2 inhibitor of the disclosure in a therapeutically effective amount does not induce cell death in greater than 10% of normal cells. A therapeutically effective amount of an EZH2 inhibitor of the disclosure does not significantly affect the viability of normal cells if administration of the compound in a therapeutically effective amount does not induce cell death in greater than 10% of normal cells.

Contacting a cell with an EZH2 inhibitor of the disclosure, or a pharmaceutically acceptable salt, prodrug, metabolite, polymorph or solvate thereof, can inhibit EZH2 activity selectively in cancer cells. Administering to a subject in need thereof an EZH2 inhibitor of the disclosure, or a pharmaceutically acceptable salt, prodrug, metabolite, polymorph or solvate thereof, can inhibit EZH2 activity selectively in cancer cells.

### EZH2 Inhibitors

EZH2 inhibitors of the disclosure comprise tazemetostat (EPZ-6438):

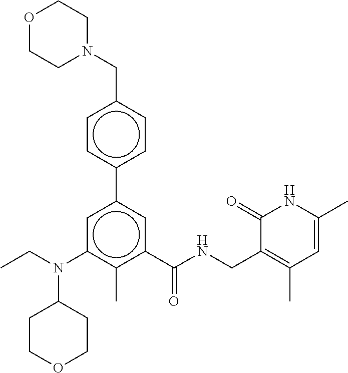

or a pharmaceutically acceptable salt thereof.

Tazemetostat is also described in U.S. Pat. Nos. 8,410,088, 8,765,732, and 9,090,562 (the contents of which are each incorporated herein in their entireties).

Tazemetostat or a pharmaceutically acceptable salt thereof, as described herein, is potent in targeting both WT and mutant EZH2. Tazemetostat is orally bioavailable and has high selectivity to EZH2 compared with other histone methyltransferases (i.e. >20,000 fold selectivity by Ki). Importantly, tazemetostat has targeted methyl mark inhibition that results in the killing of genetically defined cancer cells in vitro. Animal models have also shown sustained in vivo efficacy following inhibition of the target methyl mark. Clinical trial results described herein also demonstrate the safety and efficacy of tazemetostat.

In one embodiment, tazemetostat or a pharmaceutically acceptable salt thereof is administered to the subject at a dose of approximately 100 mg to approximately 3200 mg daily, such as about 100 mg BID to about 1600 mg BID (e.g., 100 mg BID, 200 mg BID, 400 mg BID, 800 mg BID, or 1600 mg BID), for treating a NHL. On one embodiment the dose is 800 mg BID.

EZH2 inhibitors of the disclosure may comprise, consist essentially of or consist of:

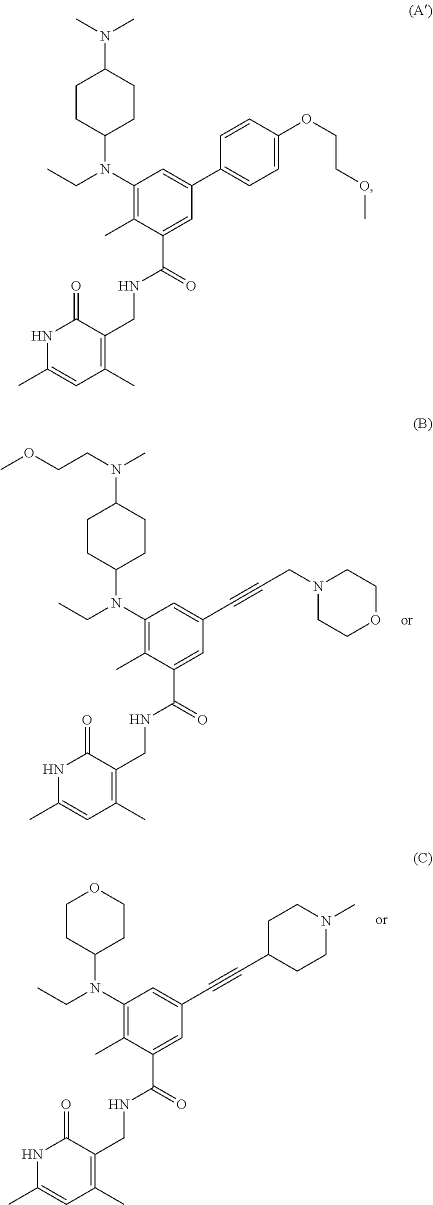

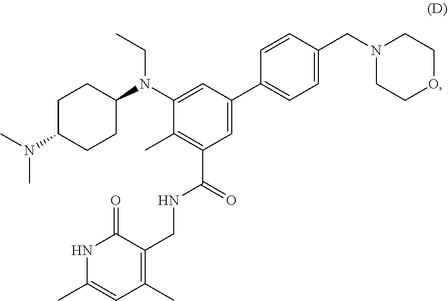 [SMILES: CCN(c1cc(-c2ccc(CN3CCOCC3)cc2)cc(C(=O)NCc2c(C)cc(C)[nH]c2=O)c1C)[C@H]1CC[C@H](N(C)C)CC1]

or stereoisomers thereof or pharmaceutically acceptable salts and solvates thereof.

EZH2 inhibitors of the disclosure may comprise, consist essentially of or consist of Compound E:

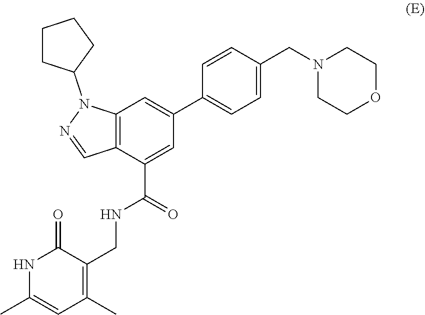 [SMILES: Cc1cc(C)c(CNC(=O)c2cc(-c3ccc(CN4CCOCC4)cc3)cc3c2cnn3C2CCCC2)c(=O)[nH]1]

or pharmaceutically acceptable salts thereof.

EZH2 inhibitors of the disclosure may comprise, consist essentially of or consist of GSK-126, having the following formula:

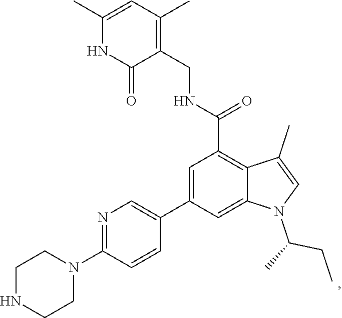 [SMILES: CC[C@H](C)n1cc(C)c2c(C(=O)NCc3c(C)cc(C)[nH]c3=O)cc(-c3ccc(N4CCNCC4)nc3)cc21]

stereoisomers thereof, or pharmaceutically acceptable salts or solvates thereof.

EZH2 inhibitors of the disclosure may comprise, consist essentially of or consist of Compound F:

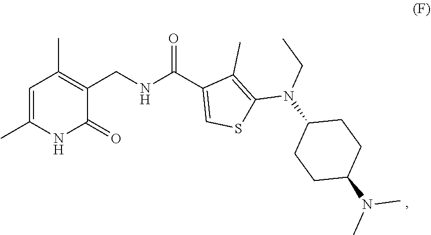

or stereoisomers thereof or pharmaceutically acceptable salts and solvates thereof.

EZH2 inhibitors of the disclosure may comprise, consist essentially of or consist of any one of Compounds Ga-Gc:

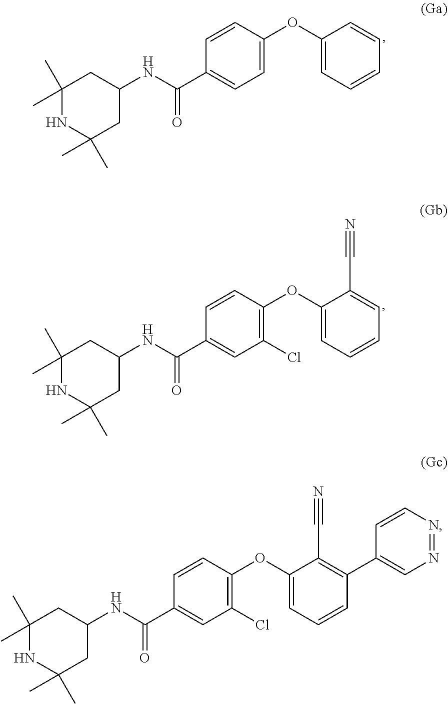 [SMILES: CC1(C)CC(NC(=O)c2ccc(Oc3ccccc3)cc2)CC(C)(C)N1 ; CC1(C)CC(NC(=O)c2ccc(Oc3ccccc3C#N)c(Cl)c2)CC(C)(C)N1 ; CC1(C)CC(NC(=O)c2ccc(Oc3cccc(-c4ccnnc4)c3C#N)c(Cl)c2)CC(C)(C)N1]

or a stereoisomer, pharmaceutically acceptable salt or solvate thereof.

EZH2 inhibitors of the disclosure may comprise, consist essentially of or consist of CPI-1205 or GSK343.

Additional suitable EZH2 inhibitors will be apparent to those skilled in the art. In some embodiments of the strategies, treatment modalities, methods, combinations, and compositions provided herein, the EZH2 inhibitor is an EZH2 inhibitor described in U.S. Pat. No. 8,536,179 (describing GSK-126 among other compounds and corresponding to WO 2011/140324), the entire contents of each of which are incorporated herein by reference.

In some embodiments of the strategies, treatment modalities, methods, combinations, and compositions provided herein, the EZH2 inhibitor is an EZH2 inhibitor described in PCT/US2014/015706, published as WO 2014/124418, in PCT/US2013/025639, published as WO 2013/120104, and in U.S. Ser. No. 14/839,273, published as US 2015/0368229, the entire contents of each of which are incorporated herein by reference

In one embodiment, the compound disclosed herein is the compound itself, i.e., the free base or “naked” molecule. In another embodiment, the compound is a salt thereof, e.g., a mono-HCl or tri-HCl salt, mono-HBr or tri-HBr salt of the naked molecule.

Compounds disclosed herein that contain nitrogens can be converted to N-oxides by treatment with an oxidizing agent (e.g., 3-chloroperoxybenzoic acid (mCPBA) and/or hydrogen peroxides) to afford other compounds suitable for any methods disclosed herein. Thus, all shown and claimed nitrogen-containing compounds are considered, when allowed by valency and structure, to include both the compound as shown and its N-oxide derivative (which can be designated as N→O or N⁺—O⁻). Furthermore, in other instances, the nitrogens in the compounds disclosed herein can be converted to N-hydroxy or N-alkoxy compounds. For example, N-hydroxy compounds can be prepared by oxidation of the parent amine by an oxidizing agent such as m-CPBA. All shown and claimed nitrogen-containing compounds are also considered, when allowed by valency and structure, to cover both the compound as shown and its N-hydroxy (i.e., N—OH) and N-alkoxy (i.e., N-OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

“Isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds disclosed herein may be depicted as different chiral isomers or geometric isomers. It should also be understood that when compounds have chiral isomeric or geometric isomeric forms, all isomeric forms are intended to be included in the scope of the disclosure, and the naming of the compounds does not exclude any isomeric forms.

Furthermore, the structures and other compounds discussed in this disclosure include all atropic isomers thereof “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine. An example of keto-enol equilibria is between pyridin-2(1H)-ones and the corresponding pyridin-2-ols, as shown below.

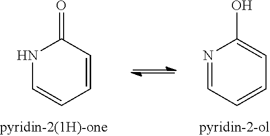 [SMILES: O=c1cccc[nH]1 ; Oc1ccccn1]

It is to be understood that the compounds disclosed herein may be depicted as different tautomers. It should also be understood that when compounds have tautomeric forms, all tautomeric forms are intended to be included in the scope of the disclosure, and the naming of the compounds does not exclude any tautomer form.

The compounds disclosed herein include the compounds themselves, as well as their salts and their solvates, if applicable. A salt, for example, can be formed between an anion and a positively charged group (e.g., amino) on an aryl- or heteroaryl-substituted benzene compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt can also be formed between a cation and a negatively charged group (e.g., carboxylate) on an aryl- or heteroaryl-substituted benzene compound. Suitable cations include sodium ion, potassium ion, magnesium ion, calcium ion, and an ammonium cation such as tetramethylammonium ion. The aryl- or heteroaryl-substituted benzene compounds also include those salts containing quaternary nitrogen atoms. In the salt form, it is understood that the ratio of the compound to the cation or anion of the salt can be 1:1, or any ration other than 1:1, e.g., 3:1, 2:1, 1:2, or 1:3.

Additionally, the compounds disclosed herein, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure, and are substituted with various groups as described herein. For example, all of the compounds represented by Formula (I) are aryl- or heteroaryl-substituted benzene compounds, and have Formula (I) as a common core.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

The present disclosure is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

### Pharmaceutical Formulations

The present disclosure also provides pharmaceutical compositions comprising at least one EZH2 inhibitor described herein in combination with at least one pharmaceutically acceptable excipient or carrier.

A “pharmaceutical composition” is a formulation containing the EZH2 inhibitors of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the disclosure includes both one and more than one such excipient. A pharmaceutical composition of the disclosure is formulated to be compatible with its intended route of administration. Examples of routes of administration include parenteral, (e.g., intravenous, intradermal, subcutaneous), and enteral routes (e.g.,oral, buccal, sublingual, sublabial), as well as administration by inhalation, transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. Suitable formulations for enteral application, e.g., for oral administration, include, for example, tablets, capsules, time-release or sustained-release tablets and capsules, powders or granules (e.g., for formulating a solution or suspension that is orally administered), syrups, solutions, or suspensions. Liquid formulations for oral administration may include one or more diluent, e.g., water, which may, in some embodiments, be sterile. Such liquid formulations for oral administration may also include a stabilizer, an antibacterial agent, an antioxidant, a chelating agent, a buffer, an agent for the adjustment of tonicity, and agents to control appearance and taste, such as a sweetener and/or a flavoring agent. Powders from which a solution or suspension can be reconstituted before oral administration may contain similar agents.

A compound or pharmaceutical composition of the disclosure can be administered to a subject in many of the well-known methods currently used for chemotherapeutic treatment. For example, for treatment of cancers, a compound of the disclosure may be injected directly into tumors, injected into the blood stream or body cavities or taken orally or applied through the skin with patches. The dose chosen should be sufficient to constitute effective treatment but not as high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of an EZH2 inhibitor, composition, or pharmaceutical composition thereof effective to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is cancer, including but not limited to, an INI1-deficient tumor.

For any EZH2 inhibitor of the disclosure, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED50 (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

Some embodiments provide pharmaceutical compositions, dosage forms, and/or methods of using such compositions or dosage forms, wherein an EZH2 inhibitor is formulated as an oral tablet, or as a suspension or solution. In some such embodiments, the EZH2 inhibitor may be formulated for administration at a dose of between 10 mg/kg/day and 1600 mg/kg/day. In some embodiments, the pharmaceutical composition, or dosage form may be administered to a subject at a dose of about 100, 200, 400, 800, or 1600 mg. In some embodiments, an EZH2 inhibitor may be formulated for administration at a dose of about 800 mg. Such formulation may comprise one or multiple dosage forms, e.g., a single tablet or capsule, or a plurality of tablets or capsules, or a certain amount of powder, solution, or suspension comprising the EZH2 inhibitor. In some embodiments, pharmaceutical compositions or dosage forms are provided in which an EZH2 inhibitor is formulated for administration once or twice per day (BID). In some embodiments, pharmaceutical compositions or dosage forms are provided in which an EZH2 inhibitor is formulated for administration at a dose of between 10 mg/kg/day and 1600 mg/kg/day BID. For example, in some embodiments, a pharmaceutical composition is provided that is suitable for administration of an EZH2 inhibitor at a dose of 800 mg BID.

In some embodiments, a pharmaceutical composition or dosage form comprising an EZH2 inhibitor is provided that is formulated for parenteral or enteral administration, for example, as an oral tablet, suspension, or solution, or as a solution or suspension for administration to the CSF by any route. In some such embodiments, the pharmaceutical composition or dosage form may be suitable for administration of the EZH2 inhibitor at a dose of between 10 mg/kg/day and 1600 mg/kg/day, e.g., at a dose of 10 mg/kg/day, 20 mg/kg/day, 25 mg/kg/day, 30 mg/kg/day, 40 mg/kg/day, 50 mg/kg/day, 60 mg/kg/day, 70 mg/kg/day, 75 mg/kg/day, 80 mg/kg/day, 90 mg/kg/day, 100 mg/kg/day, 200 mg/kg/day, 250 mg/kg/day, 300 mg/kg/day, 400 mg/kg/day, 500 mg/kg/day, 600 mg/kg/day, 700 mg/kg/day, 750 mg/kg/day, 800 mg/kg/day, 900 mg/kg/day, 1000 mg/kg/day, 1100 mg/kg/day, 1200 mg/kg/day, 1250 mg/kg/day, 1300 mg/kg/day, 1400 mg/kg/day, 1500 mg/kg/day, or 1600 mg/kg/day. For example, in some embodiments, a pharmaceutical composition or dosage form is provided that is suitable for administration of an EZH2 inhibitor at a dose of between 10 mg/kg/day and 1600 mg/kg/day BID. For example, EZH2 inhibitors of the disclosure may be administered at a dose of 800 mg BID.

In some embodiments, a pharmaceutical composition or dosage form comprising an EZH2 inhibitor is provided that is formulated for parenteral or enteral administration, for example, as an oral tablet, suspension, or solution. In some such embodiments, the pharmaceutical composition or dosage form may be suitable for administration of the EZH2 inhibitor at a dose of between 10 mg/m²/day and 1200 mg/m²/day, e.g., at a dose of 10 mg/m²/day, 20 mg/m²/day, 25 mg/m²/day, 30 mg/m²/day, 40 mg/m²/day, 50 mg/m²/day, 60 mg/m²/day, 70 mg/m²/day, 75 mg/m²/day, 80 mg/m²/day, 90 mg/m²/day, 100 mg/m²/day, 110 mg/m²/day, 120 mg/m2/day, 125 mg/m²/day, 130 mg/m²/day, 140 mg/m²/day, 150 mg/m²/day, 160 mg/m²/day, 170 mg/m²/day, 175 mg/m²/day, 180 mg/m²/day, 190 mg/m²/day, 200 mg/m²/day, 210 mg/m²/day, 220 mg/m²/day, 225 mg/m²/day, 230 mg/m²/day, 240 mg/m²/day, 250 mg/m²/day, 260 mg/m²/day, 270 mg/m²/day, 275 mg/m²/day, 280 mg/m²/day, 290 mg/m²/day, 300 mg/m²/day, 310 mg/m²/day, 320 mg/m²/day, 325 mg/m²/day, 330 mg/m²/day, 340 mg/m²/day, 350 mg/m²/day, 360 mg/m²/day, 370 mg/m²/day, 375 mg/m²/day, 380 mg/m²/day, 390 mg/m²/day, 400 mg/m²/day, 410 mg/m²/day, 420 mg/m²/day, 425 mg/m²/day, 430 mg/m²/day, 440 mg/m²/day, 450 mg/m²/day, 460 mg/m²/day, 470 mg/m²/day, 475 mg/m²/day, 480 mg/m²/day, 490 mg/m²/day, 500 mg/m²/day, 525 mg/m²/day, 550 mg/m²/day, 575 mg/m²/day, 600 mg/m²/day, 625 mg/m²/day, 650 mg/m²/day, 675 mg/m²/day, 700 mg/m²/day, 750 mg/m²/day, 800 mg/m²/day, 850 mg/m²/day, 900 mg/m²/day, or 1000 mg/m²/day. For example, in some embodiments, a pharmaceutical composition or dosage form is provided that is suitable for administration of an EZH2 inhibitor at a dose of between 10 mg/m²/day and 1200 mg/m²/day, e.g., between 100 and 300 mg/m²/day, between 200 and 300 mg/m²/day, between 200 and 400 mg/m²/day, between 250 and 500 mg/m²/day, between 150 and 400 mg/m²/day, between 150 and 300 mg/m²/day, between 300 and 600 mg/m²/day, between 350 and 400 mg/m²/day, between 350 and 700 mg/m²/day, or between 400 and 1200 mg/m²/day. For example, in some embodiments, a pharmaceutical composition or dosage form is provided that is suitable for administration of an EZH2 inhibitor 10 mg/m²/day and 1200 mg/m²/day BID. For example, EZH2 inhibitors of the disclosure may be administered at a dose of 100, 120, 140, 150, 160, 200, 240, 250, 260, 300, 320, 350, 380, 400, or 600 mg/m² BID.

In some embodiments, a pharmaceutical composition or dosage form comprising an EZH2 inhibitor is provided that is formulated for parenteral or enteral administration, for example, as an oral tablet, suspension, or solution. In some such embodiments, the pharmaceutical composition or dosage form may be suitable for administration of the EZH2 inhibitor at a dose of 50%, 60%, 70%, 80%, 90%, or any percentage in between of a value of an area under the curve (AUC) of a steady state plasma and/or CSF concentration (AUC_{SS}) of an EZH2 inhibitor, wherein the AUC_{SS} is determined following administration of the EZH2 inhibitor to an adult subject at a dose of between 10 mg/kg/day and 1600 mg/kg/day BID.

In some embodiments, a pharmaceutical composition or dosage form comprising an EZH2 inhibitor is provided that is formulated for parenteral or enteral administration, for example, as an oral tablet, suspension, or solution. In some such embodiments, the pharmaceutical composition or dosage form may be suitable for administration of the EZH2 inhibitor at a dose of between 230 mg/m² and 600 mg/m², inclusive of the endpoints. EZH2 inhibitors of the disclosure may be administered at a dose of between 300 mg/m² and 600 mg/m². EZH2 inhibitors of the disclosure may be administered at a dose of between 230 mg/m² and 305 mg/m², inclusive of the endpoints. EZH2 inhibitors of the disclosure may be administered at a dose of 240 mg/m². EZH2 inhibitors of the disclosure may be administered at a dose of 300 mg/m². EZH2 inhibitors of the disclosure may be administered once or twice per day (BID). For example, EZH2 inhibitors of the disclosure may be administered at a dose of between 230 mg/m² and 600 mg/m² BID, inclusive of the endpoints.

In some embodiments, a pharmaceutical composition or dosage form comprising an EZH2 inhibitor is provided that is suitable for administration of the EZH2 inhibitor at a dose of about 60% of the area under the curve (AUC) at steady state (AUC_{SS}) following administration of 1600 mg twice a day to an adult subject. Accordingly, in some such embodiments, a pharmaceutical composition or dosage form is provided that is suitable for administration of the EZH2 inhibitor at a dose of about about 600 mg/m² per day or at least 600 mg/m² per day. In some embodiments, the pharmaceutical composition is suitable for administration to a pediatric subject.

In some embodiments, a pharmaceutical composition or dosage form comprising an EZH2 inhibitor is provided that is suitable for administration of the EZH2 inhibitor at a dose of about 80% of the area under the curve (AUC) at steady state (AUC_{SS}) following administration of 800 mg twice a day to an adult subject. Accordingly, in some such embodiments, a pharmaceutical composition or dosage form is provided that is suitable for administration of the EZH2 inhibitor at a dose of about about 390 mg/m² per day or at least 390 mg/m² per day. In some embodiments, the pharmaceutical composition is suitable for administration to a pediatric subject.

In some embodiments, the present disclosure provides pharmaceutical compositions and dosage forms comprising an EZH2 inhibitor that are suitable for administration to a pediatric subject, e.g., a subject between 6 months and 21 years of age, inclusive of the endpoints; between 1 year and 18 years of age, inclusive of the endpoints; 10 years of age or less; 5 years of age or less; between 6 months and 1 year of age, inclusive of the endpoints; about 1 year of age; about 2 years of age; about 3 years of age; about 4 years of age; about 5 years of age; about 6 years of age; about 7 years of age; about 8 years of age; about 9 years of age; about 10 years of age; about 11 years of age; about 12 years of age; about 13 years of age; about 14 years of age; about 15 years of age; about 16 years of age; about 17 years of age; about 18 years of age; about 19 years of age; about 20 years of age; or about 21 years of age. In some embodiments, a pharmaceutical composition or dosage form comprising an EZH2 inhibitor is provided that is suitable for administration to a subject that is at least 1, 2, 3, 4, 5, 6, 7, 8, 9, 10, or 12 years of age, and not more than 5, 6, 7, 8, 9, 10, 11, 12, 13, 14 ,15, 16, 17, 18, 19, 20, or 21 years of age, wherein every possible age range that can be formed with these values (e.g., at least 4 and not more than 12 years of age; or at least 10 and not more than 18 years of age).

The pharmaceutical compositions containing an EZH2 inhibitor of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Compositions suitable for oral administration generally include an inert diluent or an edible pharmaceutically acceptable carrier. In some embodiments, they can be enclosed in capsules, e.g., in gelatin capsules, or compressed into tablets. For the purpose of oral therapeutic administration, the active compound can be incorporated with excipients and used in the form of tablets, troches, or capsules. Compositions for oral administration can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds (e.g., EZH2 inhibitors of the disclosure) can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions can be included in a container, pack, or dispenser together with instructions for administration.

The compounds of the present disclosure are capable of further forming salts. All of these forms are also contemplated within the scope of the claimed disclosure.

As used herein, “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs) as defined herein, of the same salt.

The EZH2 inhibitors of the present disclosure can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., an acetate, propionate or other ester.

The EZH2 inhibitors of the present disclosure can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compounds of the present disclosure can be delivered in prodrug form. Thus, the present disclosure is intended to cover prodrugs of the presently claimed compounds, methods of delivering the same and compositions containing the same. “Prodrugs” are intended to include any covalently bonded carriers that release an active parent drug of the present disclosure in vivo when such prodrug is administered to a subject. Prodrugs in the present disclosure are prepared by modifying functional groups present in the compound in such a way that the modifications are cleaved, either in routine manipulation or in vivo, to the parent compound. Prodrugs include compounds of the present disclosure wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl) N-Mannich bases, Schiff bases and enaminones of amino functional groups, oximes, acetals, ketals and enol esters of ketone and aldehyde functional groups in compounds of the disclosure, and the like, See Bundegaard, H., Design of Prodrugs, p1-92, Elesevier, New York-Oxford (1985).

The EZH2 inhibitors, or pharmaceutically acceptable salts, esters or prodrugs thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

The dosage regimen can be daily administration (e.g. every 24 hours) of a compound of the present disclosure. The dosage regimen can be daily administration for consecutive days, for example, at least two, at least three, at least four, at least five, at least six or at least seven consecutive days. Dosing can be more than one time daily, for example, twice, three times or four times daily (per a 24 hour period). The dosing regimen can be a daily administration followed by at least one day, at least two days, at least three days, at least four days, at least five days, or at least six days, without administration.

Techniques for formulation and administration of the disclosed compounds of the disclosure can be found in Remington: the Science and Practice of Pharmacy, 19^{th} edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight.

Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

### EXAMPLES

In order that the invention disclosed herein may be more efficiently understood, examples are provided below. It should be understood that these examples are for illustrative purposes only and are not to be construed as limiting the disclosure in any manner.

### Example 1: Tazemetostat Descreases Medulloblastoma Cell Growth

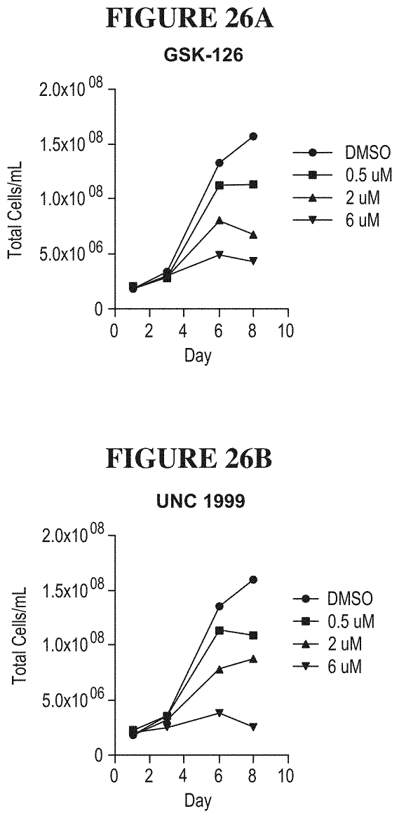

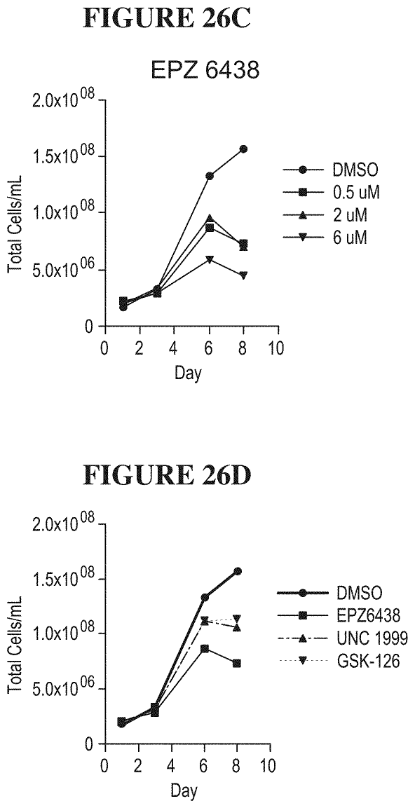

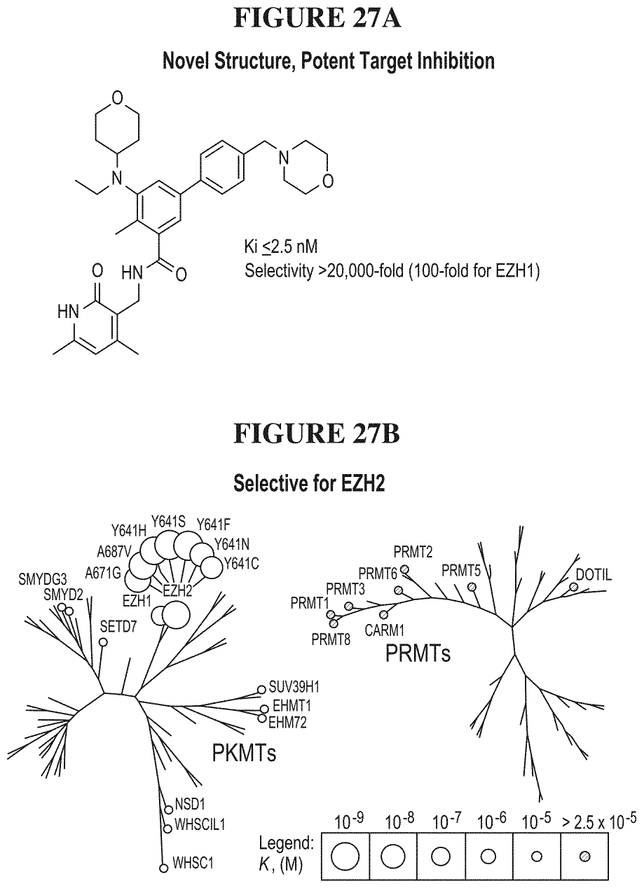

Medulloblastoma cells are treated with either a negative control (DMSO) or varying concentrations of tazemetostat (EPZ 6438): 0.5 μM, 2 μM and 6 μM. The total cells per milliliter of culture were counted each day for 10 days. While each tazemetostat treatment demonstrated a significant decrease on medulloblastoma cell growth compared to wild type (FIG. 26C), the effect was concentration dependent.

When compared to the efficacy of other small molecule EZH2 inhibitors, including GSK-126 and UNC 1999, Tazemetostat demonstrated a superior ability to decrease medulloblastoma cell growth (FIG. 26D).

### Example 2: Tazemetostat Descreases Medulloblastoma Cell Growth in an Ex Vivo Slice Culture

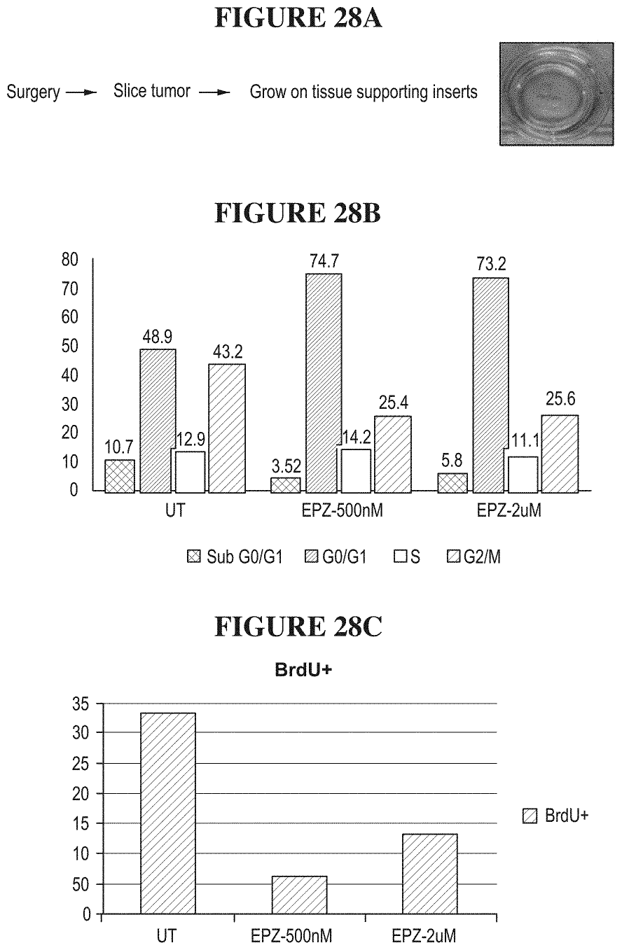

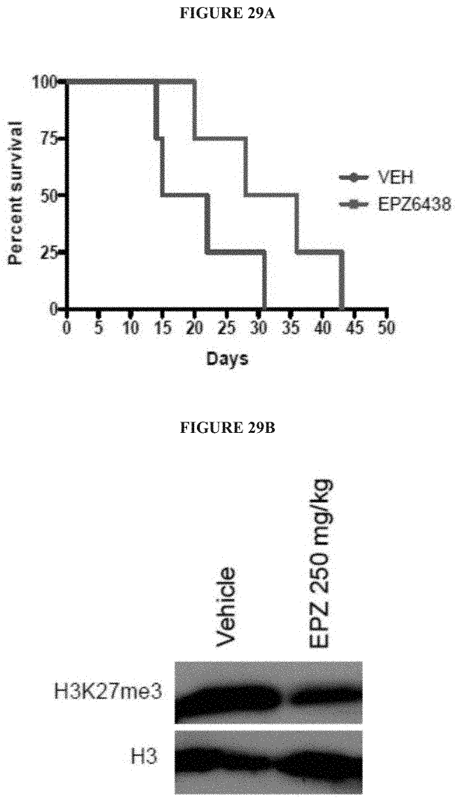

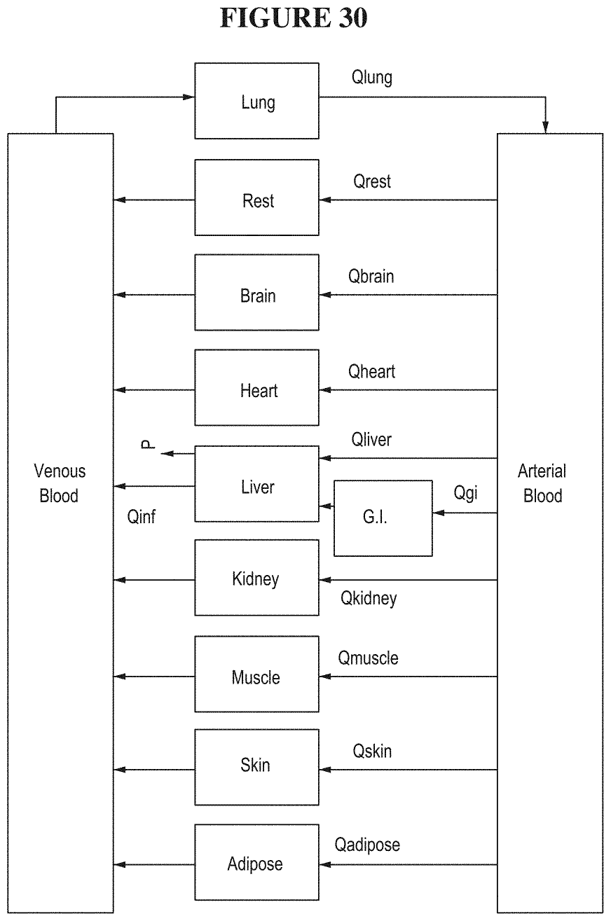

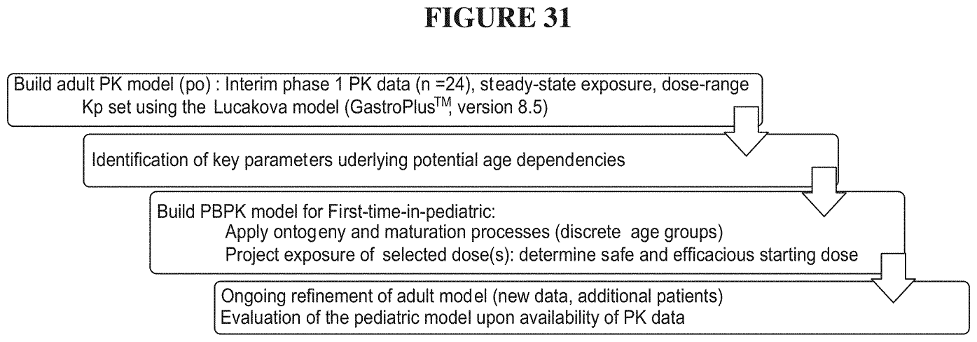

A 5 year old patient having medulloblastoma underwent surgery to remove a slice of tumor tissue for testing. The medulloblastoma slice was cultured ex vivo on tissue supporting inserts (FIG. 28A). Portions of the slice culture were untreated, treated with a lower concentration of tazemetostat (500 nM) or a higher concentration of tazemetostat (2 μM) for 4 days. Following the treatment period, the cells of the slice culture were treated with BrdU for 4 hours prior to disaggregation and sorting by flow cytometry.

FIG. 28B provides the results of the treatment by depicting the percent of cells in each of four cell cycle stages (sub G0/G1, Go/G1, S or G2/M) following each one of the treatment conditions. The data demonstrate that, compared to the untreated control, an increased proportion of medulloblastoma cells treated with tazemetostat are in the G0/G1 stage and a decreased proportion of medulloblastoma cells treated with tazemetostat are in the G2/M stage. The data indicate that treatment with tazemetostat inhibits proliferation/growth of medulloblastoma cells by interfering with cell division.

FIG. 28C confirms the results of FIG. 28B showing that the number of cells synthesizing DNA is significantly decreased in the tazemetostat-treated cells as evidenced by decreased incorporation of BrdU.

### Example 3: Tazemetostat Pharmacokinetic (PK) Data from Human Phase 1 Clinical Trial: Steady-State PK Parameters in Adults

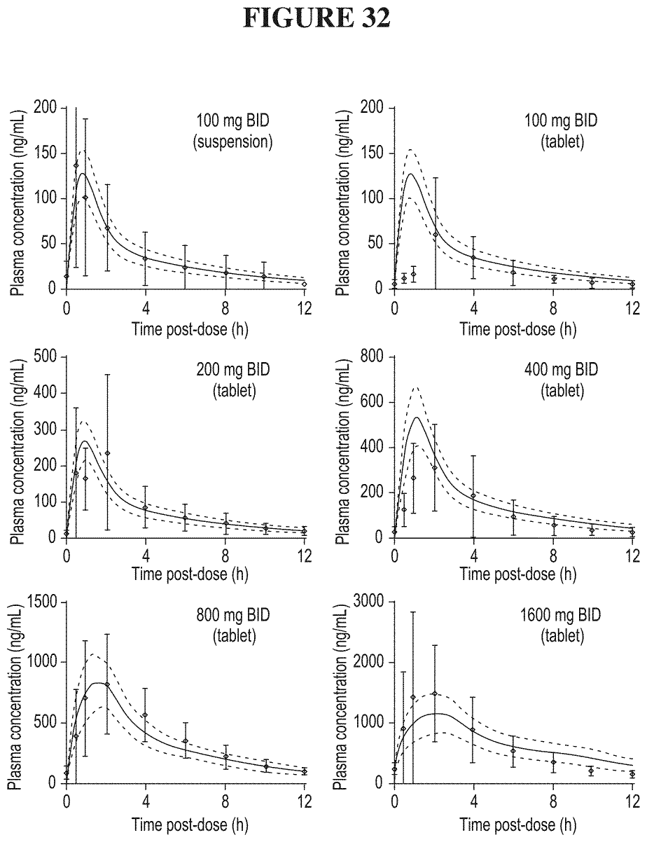

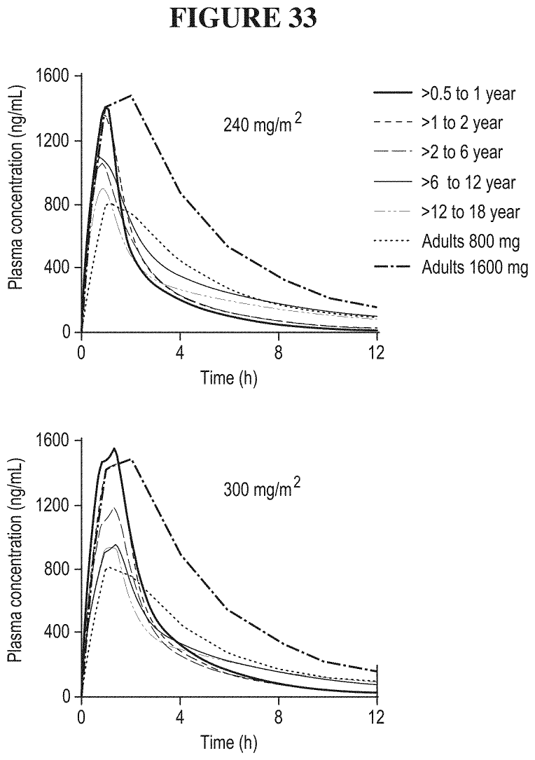

Tazemetostat pharmacokinetic (PK) data from the first in human phase 1 clinical trial study (CT.gov: NCT101897571), across a dose range of 100 mg (suspension) and 100, 200, 400, 800 and 1600 mg (tablet) p.o. twice daily (BID), together with in vitro data including plasma protein binding, blood partitioning, metabolic stability and P450 phenotyping were used to simulate adult exposures by physiologically-based pharmacokinetic (PBK) modeling (Gastroplus™ 8.5, Simulations Plus Inc.). A model fit for the adult exposures (n=24) adequately describes the time-concentration profiles of tazemetostat. This resulted in prediction of mean steady-state AUC₀₋ₜ and oral clearance (CL/F) with ±30% of the observed results across the dose ranges. In addition, mean steady-state Cₘₐₓ was predicted to ≤2-fold of the observed values, for both suspension and tablet formulations. The resultant model was used to simulate tazemetostat steady-state exposures in discrete pediatric age ranges of (6 month to 1 year (yr), >1-2 yrs, >2-6 yrs, >6-12 yrs, >12-18 yrs) following BID administration of the oral suspension. In addition to the pediatric physiology, the simulations accounted for ontogeny in hematocrit, plasma protein levels and CYP expression. Using this exposure-based analysis, pediatric doses which afforded the target AUC (80% of the adult steady-state AUCo-t at 800 mg or 300 mg/m² BID) were identified. On the body surface area normalized basis, the projected doses were comparable across the age range (1 to 18 years) from 270 to 305 mg/m² BID, with a slightly lower projected dose of 230 mg/m² BID for the 6 months to 1 year old group. As the projected doses by the age were comparable, population simulations were performed to determine the corresponding exposures for each age range at a fixed 300 mg/m² BID dose. At this dose, mean steady-state Cₘₐₓ was projected to range between 895 ng/mL and 1550 ng/mL (110% to 190% of Cₘₐₓ at 800 mg BID in adult), but within the safe and efficacious exposure range defined in adults at doses up to 1600 mg BID (FIGS. 32 and 33).

The time-concentration profiles of tazemetostat administered orally at doses 100-1600 mg BID in adults were well predicted using a PBPK model, resulting in prediction of mean steady-state AUC₀₋ₜ and oral clearance (CL/F) within ±30% of the observed results across the dose range. Using this exposure-based analysis, pediatric starting doses which afforded AUC_{SS} within the safe and efficacious exposure range defined in adults were identified. For children 1-18 years of age, the starting dose of 240 mg/m² was predicted to result in 64% and 36% of the mean steady-state (AUC_{SS}) observed for adults at 800 mg and 1600 mg doses, respectively. For children 6 months to 1 year of age, the starting dose of 240 mg/m² was predicted to result in 80% and 45% of the mean AUC_{SS} observed for adults at 800 mg and 1600 mg doses, respectively. The data demonstrated the prospective application of PBPK early in clinical development to support clinical trial design in pediatric patients (see Tables 1 and 2).

TABLE 1
Drug-Specific data input parameters used in PBPK model.

| Data inputs | Parameters |
|---|---|
| Physicochemical | pKa |
| In vitro ADME | Plasma protein binding (fᵤ) |
| | Blood:plasma partition ratio |
| | Permeability (LLC-PK1 cells) |
| | HLM clearance (Kₘ and Vₘₐₓ) |
| | CYP phenotyping |
| Clinical PK | Renal Clearance (fᵤ * GFR) |

TABLE 2
Ratio of predicted versus observed tazemetostat steady -state PK parameters in adults.

| | Cₘₐₓ (ng/mL) | | | AUC₀₋ₜ (ng*h/mL) | | | CL/F (L/h) | | |
|---|---|---|---|---|---|---|---|---|---|
| BID dose | | | Predicted/ | | | Predicted/ | | | Predicted/ |
| (mg) | Observed | Predicted | Observed | Observed | Predicted | Observed | Observed | Predicted | Observed |
| 100 (n = 6) | 99 | 147 | 1.5 | 348 | 442 | 1.3 | 309 | 225 | 0.73 |
| 200 (n = 3) | 237 | 276 | 1.2 | 1010 | 950 | 0.94 | 198 | 208 | 1.1 |
| 400 (n = 3) | 313 | 586 | 1.9 | 1510 | 2010 | 1.3 | 264 | 198 | 0.75 |
| 800 (n = 6) | 818 | 938 | 1.1 | 4630 | 4140 | 0.89 | 152 | 190 | 1.2 |
| 1600 (n = 6) | 1480 | 1230 | 0.83 | 7880 | 8200 | 1.0 | 180 | 193 | 1.1 |

The details of one or more embodiments of the disclosure are set forth in the accompanying description above. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs.

All patents and publications cited in this specification are incorporated by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow. Where names of cell lines or genes are used, abbreviations and names conform to the nomenclature of the American Type Culture Collection (ATCC) or the National Center for Biotechnology Information (NCBI), unless otherwise noted or evident from the context.

The invention disclosed herein can be embodied in other specific forms without departing from the spirit or essential characteristics thereof. The foregoing description has been presented only for the purposes of illustration and is not intended to limit the invention to the precise form disclosed, but by the claims appended hereto, and all changes that come within the meaning and range of equivalency of the claims are intended to be embraced therein.

1. A method of treating cancer in a pediatric subject in need thereof comprising administering to the subject a therapeutically-effective amount of an enhancer of a zeste homolog 2 (EZH2) inhibitor.

2. The method of claim 1, wherein the EZH2 inhibitor comprises 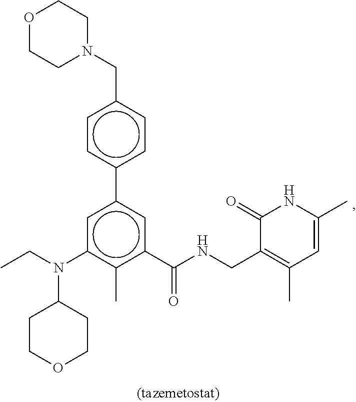
or a pharmaceutically-acceptable salt thereof.

3.-7. (canceled)

8. The method of claim 1, wherein the EZH2 inhibitor is administered orally.

9. (canceled)

10. The method of claim 8, wherein the EZH2 inhibitor is administered at a dose of between 10 mg/kg/day and 1600 mg/kg/day.

11. The method of claim 10, wherein the EZH2 inhibitor is administered at a dose of about 100, 200, 400, 800, or 1600 mg/kg/day.

12. The method of claim 11, wherein the EZH2 inhibitor is administered at a dose of about 800 mg/kg/day.

13. (canceled)

14. The method of claim 1, wherein the EZH2 inhibitor is formulated for administration to cerebral spinal fluid (CSF).

15. The method of claim 14, wherein the EZH2 inhibitor is administered to cerebral spinal fluid by an intraspinal, an intracranial, an intrathecal or an intranasal route.

16. The method of claim 8, wherein the EZH2 inhibitor is administered at a dose of between 230 mg/m² and 600 mg/m² twice per day (BID), inclusive of the endpoints, or at a dose of between 230 mg/m² and 305 mg/m² twice per day (BID), inclusive of the endpoints.

17. (canceled)

18. The method of claim 8, wherein the EZH2 inhibitor is administered at a dose of 240 mg/m² twice per day (BID), or at a dose of 300 mg/m² twice per day (BID).

19. (canceled)

20. The method of claim 8, wherein the EZH2 inhibitor is administered at a dose corresponding to about 60% of the area under the curve (AUC) of a steady state plasma concentration (AUC_{SS}) following administration of 1600 mg twice a day to an adult subject.

21. The method of claim 20, wherein the EZH2 inhibitor is administered at a dose of about 600 mg/m² per day.

22. (canceled)

23. The method of claim 8, wherein the EZH2 inhibitor is administered at a dose corresponding to about 80% of the area under the curve (AUC) of a steady state plasma concentration (AUC_{SS}) following administration of 800 mg twice a day to an adult subject.

24. The method of claim 23, wherein the EZH2 inhibitor is administered at a dose of about 390 mg/m² twice per day (BID).

25. (canceled)

26. The method of claim 8, wherein the EZH2 inhibitor is administered at a dose of between 300 mg/m² and 600 mg/m² twice per day (BID).

27. The method of claim 1, wherein the EZH2 inhibitor is administered twice per day (BID).

28. (canceled)

29. The method of claim 1, wherein the subject is between 6 months and 21 years of age, inclusive of the endpoints, or between 1 year and 18 years of age, inclusive of the endpoints.

30. (canceled)

31. The method of claim 1, wherein the subject is 10 years of age or less or 5 years of age or less.

32. (canceled)

33. The method of claim 1, wherein treating comprises preventing and/or inhibiting proliferation of a cancer cell.

34. A method of treating cancer in a subject in need thereof comprising administering to the subject a therapeutically-effective amount of tazemetostat,
wherein the therapeutically effective amount is at least 300 mg/m² twice per day (BID), and
wherein the subject is between 6 months and 21 years of age, inclusive of the endpoints.
